(19) United States
(12) Patent Application Publication
(10) Pub. No.: US 2022/0213135 A1
(43) Pub. Date: Jul. 7, 2022

(54) DERIVATIVES OF GLYCERO-MANNO-HEPTOSE PHOSPHATE AND THEIR USE IN MODULATING AN IMMUNE RESPONSE

(71) Applicant: Shanghai Yao Yuan Biotechnology Co., Ltd., Shanghai (CN)

(72) Inventors: Tian Xu, Shanghai (CN); Cong Xu, Shanghai (CN); Danyang Liu, Shanghai (CN); Jieqing Fan, Shanghai (CN)

(21) Appl. No.: 17/594,644

(22) PCT Filed: Apr. 24, 2020
(86) PCT No.: PCT/CN2020/086688

§ 371 Date: Oct. 25, 2021

(30) Foreign Application Priority Data
Apr. 26, 2019 (CN) PCT/CN2019/084582

(51) Int. Cl. C07H 11/04

(52) U.S. Cl. CPC C07H 11/04

(57) ABSTRACT

The disclosure provides compounds of formula (I),

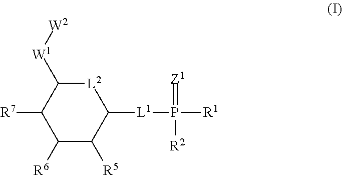

wherein R1, R2, R5, R6, R7, L1, L2, W1, W2, and Z1 are as defined herein, and compositions comprising same, and methods related to activating alpha-kinase 1 (ALPK1) for modulating an immune response and treating or preventing cancer, infection, inflammation and related diseases and disorders as well as potentiating an immune response to a target antigen.

### FIELD OF THE INVENTION

The present invention relates to compounds that are derivatives of certain bacterial metabolites in the ADP-heptose biosynthetic pathway, compositions comprising same, and methods for their use in therapy.

### BACKGROUND OF THE INVENTION

The studies on mechanism of inflammatory response have identified various protein kinases that act as essential signaling components. Defects in protein kinase are frequently associated with the pathogenesis of human inflammatory diseases, cancer and diabetes.

Alpha-kinases are a unique protein kinase superfamily, displaying little sequence similarity to typical protein kinases. A total of six alpha kinase members including alpha-protein kinase 1 (ALPK1), ALPK2, ALPK3, elongated factor-2 kinase (eEF2K), and transient receptor potential cation channel M6 and M7 (TRPM6 and TRPM7) have been identified (Ryazanov A G et al., Curr Biol 1999 9(2):R43-45; Ryazanov A G et al., Proc Natl Acad Sci USA 1997 94(10):4884-4889). ALPK1 was initially identified as a new component of raft-containing sucrose-isomerase (SI) vesicles in epithelial cells (Heinet M et al., J. Biol. Chem. 2005 280(27): 25637-43). It was shown that ALPK1 phosphorylates myosin 1 and plays an essential role in the exocytic transport to the apical plasma membrane. A transposon-inserted homozygous inactivating mutation of ALPK1 in mice resulted in motor coordination deficits which could be rescued by overexpressing full-length ALPK1 (Chen M et al., BMC Neurosci. 2011 12:1).

Genetic association studies implicated ALPK1 in risk for gout, chronic kidney disease, myocardial infarction, and diabetes (Wang S J et al., J. Mol. Med. 2011 89:1241-51; Ko A M et al., J. Intl. Epidemiol. 2013 42: 466-474; Chiba T et al., Human Cell 2015 28:1-4; Yamada Y et al. J Med Genet 2013 50:410-418; Fujimaki T et al., Biomed Report 2014 2:127-131; Shimotaka S et al., Biomed Report 12013 940-44; Yamada Y et al., Biomed. Report 2015 DOI: 10.3892/br.2015.439).

ALPK1 activation has also been implicated in cancer, including lung, colorectal, and breast cancers (Liao et al. Scientific Reports 2016 6:27350; Strietz et al., Oncotarget 2016 1-16).

Recent studies have implicated ALPK1 as an important regulator of the innate immune response activated by certain bacteria. For example, APLK1 was suggested to be a key regulator of innate immunity against bacteria through its promotion of TIFA oligomerization and interleukin 8 (IL-8) expression in response to infection with S. flexneri, S. typhimurium, and Neisseria meningitides (Milivojevic et al., PLoS Pathog 2017 13(2): e1006224). Zimmerman et al. describe an ALPK1 and TIFA dependent innate immune response triggered by the Helicobacter pylori Type IV Secretion System. (Zimmermann et al., Cell Reports 2017 20(10): 2384-95). Both of these studies suggest that the bacterial metabolite, heptose-1,7-bisphosphate (HBP) activates TIFA-dependent innate immunity.

There are many diseases, disorders, and conditions whose clinical manifestations result from inflammation and various infections. There is a need for new methods for modulating inflammation in target tissues for treating such diseases, disorders, and conditions. The present disclosure addresses this need by providing compounds that are derivatives of certain metabolites downstream from HBP in the ADP-heptose biosynthetic pathway.

### SUMMARY OF THE INVENTION

The present invention is based, in part, on the discovery that certain derivatives of the bacterial metabolite D-glycero-β-D-manno-heptose-1-phosphate (HMP1BP) possess unexpected biological activity. HMP1BP is downstream of D-glycero-β-D-manno-heptose 1,7-bisphosphate (heptose 1,7 bisphosphate or “HBP”) in the E. coli H1b-ADP biosynthetic pathway, shown in FIG. 1.

The present disclosure provides compounds represented by formula (I), or a stereoisomer, a stable isotope, prodrug or pharmaceutically acceptable salt thereof, having improved chemical and/or biological properties compared to a reference compound, for example compared to HMP1BP.

Accordingly, the present disclosure provides compounds, compositions comprising same, including pharmaceutical compositions, and methods related to modulating an immune response, treating cancer, potentiating an immune response to a target antigen, treating a liver disease or disorder including non-alcoholic steatohepatitis (NASH) and diseases and disorders caused by the hepatitis C virus (HCV) and the hepatitis B virus (HBV), and treating or preventing a disease or disorder caused by an infectious agent as described herein through administration of a compound represented by formula I, including compounds of formulas I, Ia, Ib, Ic, and Id described herein. In some embodiments, the disclosure provides methods of modulating an immune response in a subject, the methods comprising administering to the subject a composition comprising a compound represented by formulas I, Ia, Ib, Ic, and Id described herein.

The present disclosure provides compounds represented by formula (I), or a stereoisomer, a stable isotope, prodrug or pharmaceutically acceptable salt thereof:

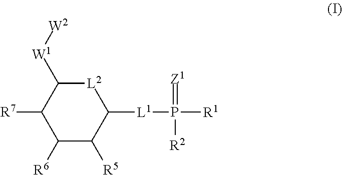

and/or a stereoisomer, tautomer, stable isotopes, prodrug or pharmaceutically acceptable salt thereof, wherein:
L¹ is selected from O, S, CH₂, CHF, CF2, OCH₂, SCH₂, OCHF, SCHF, OCF₂ or SCF₂;
L² is selected from the group consisting of O, S, CH₂, NR, CH₂, CH(OH), CHF and CF₂, wherein R is H or C1-C8 alkyl substituted with 0-3 substituents selected from halo, —OH, ═O, C1-C4 alkoxy, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members;
Z¹ is selected from O and S;
W¹ is —C(R¹⁰R¹¹)—, wherein R¹⁰ and R¹¹ are independently selected from H, D, —OH, halogen, and optionally substituted groups selected from C1-C4 alkyl, C1-C4 alkoxyl, C1-C4 haloalkyl, C1-C4-haloalkoxy, C1-C4 alkenyloxy, aralkyloxy, and 1-6 membered oligopeptidyl linked via C-termional C(O)O— and R¹²CO₂—, wherein R¹² is selected from C1-C20 alkyl, C1-C20 alkenyl, C1-C20 alkoxy, C1-C20 alkenyloxy, C1-C20 alkylamino, C3-C6 cycloalkyl, heterocyclyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members and 1-6 membered oligopeptidyl linked via N-terminal N; wherein the optional substituents for R¹⁰ and R¹¹ are 1-3 substituents independently selected from D, halogen, —OH, ═O, C1-C4 alkyl and C1-C4 alkoxy;
W² is R¹³-Q¹-W³—, wherein Q¹ is selected from —O— or —NH—; W³ is selected from a bond or C1-C3 alkylene groups optionally substituted with 1-3 substituents independently selected from halogen, —OH, ═O, C1-C3 alkoxy, C1-C3 haloalkyl, C1-C3 haloalkoxyl, C1-C3 alkenyloxy; wherein R¹³ is 1-6 membered oligopeptidyl linked via C-terminal carbonyl group or R¹⁴Q²C(O)—; wherein Q² is a bond, —O— or —NH—; R¹⁴ is 1-6 membered oligopeptidyl linked via N-terminal N or an optionally substituted group selected from C1-C20 alkyl, C1-C20 alkylenyl, C1-C20 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members, and R¹⁴ is R¹⁵-Q³-Q⁴-Q⁵-; wherein Q³, Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;
R¹ and R² are independently selected from the group consisting of —OR^{a}, and —NR^{b}R^{c}; when both R¹ and R² are —OR^{a}, the R^{a} moieties can combine to form a five or six-membered heterocyclic ring, wherein
   the five or six-membered heterocyclic ring is substituted with from 0 to 3 R³ moieties selected from the group consisting of H, D, halogen, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members, wherein the aryl or the 5 or 6 membered heteroaryl are substituted with 0 to 3 R^{3a} substituents selected from the group consisting of halogen and C1-C8 alkyl; or
   when two R³ substituents are on adjacent ring vertices of the five or six-membered heterocyclic ring, they can combine to form a fused phenyl ring, which is substituted with from 0 to 3 R⁴ moieties selected from the group consisting of H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R^{a} is selected from the group consisting of H, D, C1-C12 alkyl, C1-C12 haloalkyl,
   —C(R^{a1})(R^{a2})C(O)OR^{a3}, —C(R^{a1})(R^{a2})OC(O)R^{a3}, 3 to 6 membered heterocycloalkyl having 1-3 heteroatoms selected from N, O, and S as ring members, aryl, 5 to 10 membered heteroaryl, —C1-C4 alkylene-aryl, and —C1-C4 alkylene-5 to 10 membered heteroaryl,
   wherein the 5 or 10 membered heteroaryl has 1-3 heteroatoms selected from the group consisting of O, N, and S as ring members and the 5 or 10 membered heteroaryl is substituted with from 0 to 2 substituents selected from the group consisting of halogen, C1-C8 alkyl, and —NO₂.
each R^{b} and R^{c} are independently selected from the group consisting of H, C1-C12 alkyl, C1-C12 alkoxyl, C1-C12 alkanoyloxyl, C1-C12 alkenyloxyl, C3-C6 cycloalkyl, 4 to 6 membered heterocyclyoalkyl having 1-3 heteroatoms selected from N, O and S as ring members, aryl, 5 to 10 membered heteroaryl having 1-3 heteroatoms selected from N, O and S as ring members, and —C(R^{b1})(R^{b2})C(═O)OR^{b3};
each R^{a1}, R^{a2}, R^{b1}, and R^{b2} is selected from the group consisting of H, D, and C1-C4 alkyl C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocycloalkyl having 1-3 heteroatoms selected from N, O and S as ring members, aryl, and 5 to 10 membered heteroaryl having 1-3 heteroatoms selected from N, O and S as ring members;
each R^{a3} and R^{b3} is independently H, D, C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members; and
R⁵, R⁶ and R⁷ are independently selected from H, —OH, halogen, and R¹²CO₂—, and at least two of R⁵, R⁶ and R⁷ are —OH or R¹²CO₂—, wherein R¹² is selected from C1-C8 alkyl, C1-C8 alkoxyl, C1-C8 alkanoyloxyl, C1-C8 alkenyloxyl, C1-C8 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members; wherein any two of the adjacent groups of R⁵, R⁶ and R⁷ can cyclize to form heterocycloalkyl containing 5 to 9 ring members and having 1-3 heteroatoms selected from N, O and S as ring members, each substituted by 0-3 substituents independently selected from D, CN, halogen, —OH, ═O, C1-C4 alkyl and C1-C4 alkoxy.

In embodiments, the disclosure provides a pharmaceutical composition comprising a compound of formula (I) as described herein, and a pharmaceutically acceptable carrier.

In embodiments, the disclosure provides a method for modulating an immune response in a subject in need of such treatment, the method comprising administering to the subject a composition comprising a compound of formulas I, Ia, Ib, Ic, and Id described herein, and prodrugs, analogs and derivatives thereof. In embodiments, the method for modulating an immune response is selected from activation of innate immunity and activation of adaptive immunity.

In embodiments, the disclosure provides a method for treating cancer in a subject in need of such treatment, the method comprising administering to the subject a composition comprising a compound of formulas I, Ia, Ib, Ic, and Id described herein, and prodrugs, analogs and derivatives thereof. In embodiments, the cancer is selected from soft tissue sarcoma, breast cancer, head and neck cancer, melanoma, cervical cancer, bladder cancer, hematologic malignancy, glioblastoma, pancreatic cancer, prostate cancer, colon cancer, breast cancer, renal cancer, lung cancer, merkel cell carcinoma, small intestine cancer, thyroid cancer, acute myelogenous leukemia (AML), acute lymphocytic leukemia (ALL), chronic lymphocytic leukemia (CLL), chronic myelogenous leukemia (CML), gastric cancer, gastrointestinal stromal tumors, non-Hodgkins lymphoma, Hodgkins lymphoma, liver cancer, leukemia, lymphoma, T-cell lymphoma, brain cancer, and multiple myeloma. In embodiments, the cancer is selected from breast cancer, head and neck cancer, melanoma, renal cancer, lung cancer, merkel cell carcinoma, and lymphoma.

In embodiments, the disclosure provides a method for potentiating an immune response to a target antigen in a subject, the method comprising administering to the subject a composition comprising a compound of formulas I, Ia, Ib, Ic, and Id described herein, and prodrugs, analogs and derivatives thereof, as a vaccine or immunologic adjuvant that acts to potentiate an immune response to the target antigen. In embodiments, the target antigen is an antigen of an infectious agent selected from the group consisting of adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), rotavirus, Salmonella typhi, Staphylococcus aureus, Streptococcus pyogenes, varicella, West Nile virus, Yersinia pestis, and Zika virus. In embodiments, a compound of formula 1 as described herein, acts as a vaccine adjuvant for a vaccine in the treatment or prevention of anthrax, caries, Chagas disease, dengue, diphtheria, ehrlichiosis, hepatits A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, or visceral leishmaniasis.

In embodiments, the disclosure provides a method for treating a disease or disorder amendable to treatment by activation of NFkB, p38, and JNK cell signaling pathways in cells of a subject, the method comprising administering to the subject a composition comprising a compound of formulas I, Ia, Ib, Ic, and Id described herein, and prodrugs, analogs and derivatives thereof. In embodiments, the disease or disorder is selected from tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis bullous diseases, and diseases and disorders caused by the hepatitis C virus (HCV), the hepatitis B virus (HBV), or the human immunodeficiency virus (HIV).

In embodiments, the disclosure provides a method for treating or preventing a disease or disorder caused by an infectious agent selected from a bacteria, virus, or parasite in a subject in need thereof, the methods comprising administering to the subject a composition comprising a compound of formulas I, Ia, Ib, Ic, and Id described herein, and prodrugs, analogs and derivatives thereof. In embodiments, the infectious agent is a bacteria. In embodiments, the infectious agent is a virus. In embodiments, the infectious agent is a parasite. In embodiments, the bacteria is a Gram-negative or a Gram-positive bacteria. In embodiments, the Gram-negative bacteria is selected from the group consisting of Acinetobacter baumanii, Aggregatobacter actinomycetemcomitans, Bartonella bacilliformis, Bartonella henselae, Bartonella quintana, Bifidobacterium, Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trachomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B. typhus, Salmonella dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus and Yersinia pestitis. In embodiments, the Gram-positive bacteria selected from the group consisting of Actinomycetes, Bacillus anthracis, Bacillus subtilis, Clostridium tetani, Clostridium perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin-resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, and Streptococcus mutants. In embodiments, the virus is selected from the group consisting of ebolavirus, hepatitis B virus, hepatitis C virus, herpes simplex virus, human immunodeficiency virus (HIV), human papillomavirus (HPV-6, HPV-11), human SARS coronavirus, influenza A virus, influenza B virus, influenza C virus, measles virus, rabies virus, poliovirus, SARS corona virus, and yellow fever virus. In embodiments, the parasite is selected from the group consisting of Acanthamoeba spp, American trypanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, Dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In embodiments of any of the foregoing methods, the method may further comprise administering to the subject one or more additional therapeutic agents or immune modulators, and combinations thereof. In embodiments, the one or more additional therapeutic agents is selected from an anti-microbial agent, such as an anti-bacterial agent, an anti-viral agent, or an anti-parasitic agent, an anti-cancer agent, or a therapeutic agent for the treatment of tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, and ankylosing spondylitis bullous diseases.

In embodiments of the methods for treating cancer, the one or more additional therapeutic agents is an immune modulator. In embodiments, the immune modulator is selected from one or more of an inhibitor or antagonist of an immune checkpoint regulator, an immune stimulatory molecule, and an agonist of an immune co-stimulatory molecule. In embodiments, the inhibitor or antagonist of an immune checkpoint regulator is a PD-1/PD-L1 inhibitor. In embodiments, the PD-1/PD-L1 inhibitor is selected from the group consisting of nivolumab, pembrolizumab, pidilizumab, BMS-936559, atezolizumab, durvalumab, and avelumab. In embodiments, the immune modulator is selected from interferon alpha (INFα), a stimulator of interferon genes (“STING”) agonist, a TLR agonist (e.g., resquimod), and an anti-OX40 (CD134) agonist antibody. In embodiments, the agonist of an immune co-stimulatory molecule is an anti-OX40 (CD134) agonist antibody. In embodiments, the cancer is selected from advanced melanoma, non-small cell lung cancer, renal cell carcinoma, bladder cancer, Hodgkin's lymphoma, liver cancer, gastric cancer, colon cancer, breast cancer, non-Hodgkin's lymphoma, prostate cancer, head and neck cancer, thyroid cancer, brain cancer, acute myeloid leukemia (AML), merkel cell carcinoma, multiple myeloma, cervical cancer, and sarcoma.

In embodiments, the one or more additional immune modulators is an inhibitor or antagonist of an immune checkpoint regulator, or a vaccine against an immune checkpoint regulator. In embodiments, the one or more additional immune modulators is an agonist of an immune an immune checkpoint regulator, such as a co-stimulatory molecule, for example an agonist of OX40 (CD134). In embodiments, the immune checkpoint regulator is selected from the programed cell death 1 (PD-1) receptor (CD279), a ligand of PD-1 (e.g., PD-L1), cytotoxic T-lymphocyte associated protein 4 (CTLA4), tumor necrosis factor receptor superfamily member 9 (alternatively TNFRSF9, 4-1BB) and 4-1BB ligands, tumor necrosis factor receptor superfamily member 4 (alternatively TNFRSF4, OX40) and OX40 ligands, glucocorticoid-induced TNFR-related protein (GITR), Tumor Necrosis Factor Receptor Superfamily Member 7 (alternatively TNFRSF7, cluster of differentiation 27, CD27), TNFRSF25 and TNF-like ligand 1A (TL1A), TNF Receptor Superfamily Member 5 (alternatively TNFRSF5, CD40) and CD40 ligand, Herpesvirus entry mediator (HVEM)-tumor necrosis factor ligand superfamily member 14 (alternatively TNFSF14, LIGHT)-lymphotoxin alpha (LTA), herpesvirus entry mediator-(HVEM)-B- and T-lymphocyte attenuator (BTLA)-CD160 (alternatively TNFSF14), lymphocyte activating gene 3 (LAG3), T-cell immunoglobulin and mucin-domain containing-3 (TIM3), sialic-acid-binding immunoglobulin-like lectins (SIGLECs), inducible T-cell costimulator (ICOS) and ICOS ligand, B7-H3 (B7 family, alternatively CD276), V-set domain-containing T-cell activation inhibitor 1 (VTCN1, alternatively B7-H4), V-Type immunoglobulin domain-containing suppressor of T-cell activation (VISTA), human endogenous retrovirus-H long terminal repeat-associating protein 2 (HHLA2)-transmembrane and Immunoglobulin domain containing 2 (TMIGD2), butyrophilins, natural killer cell receptor 2B4 (alternatively NKR2B4, CD244) and B-Cell Membrane Protein (CD48), T-Cell Immunoreceptor with Immunoglobulin (Ig) and immunoreceptor tyrosine-based inhibition motif domains (TIGIT) and Poliovirus receptor (PVR) family members, killer-cell immunoglobulin-like receptors (KIRs), Immunoglobulin-like transcripts (ILTs) and leukocyte immunoglobulin-like receptor (LIRs), natural killer group protein 2 member D (NKG2D) and natural killer group protein 2 member A (NKG2A), major histocompatibility complex (MHC) class I polypeptide-related sequence A (MICA) and MHC class I polypeptide-related sequence B (MICB), natural killer cell receptor 2B4 (CD244), colony stimulating factor 1 receptor (CSF1R), indoleamine 2,3-dioxygenase (IDO), transforming growth factor beta (TGFβ), Adenosine-ecto-nucleotidase triphosphate diphosphohydrolase 1 (CD39)-5′-nucleotidase (CD73), C—X—C motif chemokine receptor 4 (CXCR4) and C—X—C motif chemokine ligand 12 (CXCL12), phosphatidylserine, signal regulatory protein alpha (SIRPA) and integrin associated protein (CD47), vascular endothelial growth factor (VEGF), and neuropilin.

In embodiments, the one or more additional immune modulators is a vaccine.

In embodiments of a method for treating cancer, the vaccine is a vaccine against a tumor antigen. In embodiments, the tumor antigen is selected from glycoprotein 100 (gp100), mucin 1 (MUC1), and melanoma-associated antigen 3 (MAGEA3).

In embodiments, the one or more additional immune modulators is a T cell, preferably a chimeric antigen receptor T cell. In embodiments, the one or more additional immune modulators is a recombinant protein, preferably selected from granulocyte-macrophage colony-stimulating factor (GM-CSF), interleukin 7 (IL-7), IL-12, IL-15, IL-18, and IL-21.

In embodiments of any of the foregoing methods, the composition may comprise a compound of formulas I, Ia, Ib, Ic, and Id described herein, and prodrugs, analogs and derivatives thereof.

In embodiments, the disclosure provides a method for treating a liver disease or disorder in a subject in need of such treatment, the method comprising administering to the subject a compound of formulas I, Ia, Ib, Ic, and Id described herein, and prodrugs, analogs and derivatives thereof. In embodiments, the liver disease or disorder is selected from liver cancer, non-alcoholic steatohepatitis (NASH), and a disease or disorder caused by infection with the hepatitis C virus (HCV) or the hepatitis B virus (HBV).

In embodiments of any of the foregoing methods, the subject may be a vertebrate. In embodiments, the subject is a human.

The disclosure also provides a vaccine composition or vaccine adjuvant composition comprising a compound of formulas I, Ia, Ib, Ic, and Id described herein, and prodrugs, analogs and derivatives thereof, and a carrier.

In embodiments, the disclosure provides a vaccine composition or vaccine adjuvant composition comprising a compound of formulas I, Ia, Ib, Ic, and Id described herein, and prodrugs, analogs and derivatives thereof.

In embodiments, the disclosure provides a pharmaceutical composition comprising a compound of formulas I, Ia, Ib, Ic, and Id described herein, and prodrugs, analogs and derivatives thereof.

In embodiments, the disclosure provides a method of treating cancer in a subject in need of such treatment, comprising administering to the subject a composition comprising a compound of formulas I, Ia, Ib, Ic, and Id described herein, and prodrugs, analogs and derivatives thereof. In embodiments, the method further comprises administering to the subject a PD-1/PD-L1 inhibitor or an agonist of an immune co-stimulatory molecule. In embodiments, the PD-1/PD-L1 inhibitor is selected from the group consisting of nivolumab, pembrolizumab, pidilizumab, BMS-936559, atezolizumab, durvalumab, and avelumab. In embodiments, the agonist of an immune co-stimulatory molecule is an anti-OX40 (CD134) agonist antibody. In accordance with the foregoing methods, the subject may be a human subject and the cancer may be a cancer as described hereinabove. In embodiments, the cancer is a solid tumor. In embodiments, the cancer is refractory.

The disclosure further provides a composition for use in therapy, the composition comprising a compound of formulas I, Ia, Ib, Ic, and Id described herein, and prodrugs, analogs and derivatives thereof.

The disclosure also provides a composition for use in a method for modulating an immune response in a subject in need of such treatment, the composition comprising a compound of formulas I, Ia, Ib, Ic, and Id described herein, and prodrugs, analogs and derivatives thereof.

The disclosure also provides a composition for use in a method for treating cancer in a subject in need of such treatment, the composition comprising a compound of formulas I, Ia, Ib, Ic, and Id described herein, and prodrugs, analogs and derivatives thereof.

The disclosure also provides a composition for use in a method for potentiating an immune response in a subject in need of such treatment, the composition comprising a compound of formulas I, Ia, Ib, Ic, and Id described herein, and prodrugs, analogs and derivatives thereof.

The disclosure also provides a composition for use in a method for treating a disease or disorder amendable to treatment by activation of NFkB, p38, and JNK cell signaling pathways in cells of a subject in a subject in need of such treatment, the composition comprising a compound of formulas I, Ia, Ib, Ic, and Id described herein, and prodrugs, analogs and derivatives thereof.

The disclosure also provides a composition for use in treating or preventing a disease or disorder caused by an infectious agent selected from a bacteria, virus, or parasite in a subject in need thereof, the composition comprising a compound of formulas I, Ia, Ib, Ic, and Id described herein, and prodrugs, analogs and derivatives thereof.

The disclosure also provides a composition for use in a method for treating cancer in a subject in need of such treatment, the composition comprising a compound of formulas I, Ia, Ib, Ic, and Id described herein, and prodrugs, analogs and derivatives thereof, and the method comprising combination therapy of the ALPK1 agonist with an immune modulator selected from one or more of an inhibitor or antagonist of an immune checkpoint regulator, an immune stimulatory molecule, and an agonist of an immune co-stimulatory molecule.

The disclosure also provides a composition for use in a method for treating a liver disease or disorder in a subject in need of such treatment, the composition comprising a a compound of formulas I, Ia, Ib, Ic, and Id described herein, and prodrugs, analogs and derivatives thereof, wherein the liver disease or disorder is optionally selected from liver cancer, non-alcoholic steatohepatitis (NASH), and a disease or disorder caused by infection with the hepatitis C virus (HCV) or the hepatitis B virus (HBV).

### BRIEF DESCRIPTION OF THE FIGURES

FIG. 1. Schematic of bacterial H1b-ADP-biosynthetic pathway.

FIG. 2. Compound 2 has unexpected biological activity in liver cells compared to H1BADP (Compound 1). Primary hepatocytes were isolated from fresh mouse livers of C57/b6 background. Cells were cultured in serum-free medium overnight before treatment with either Compound 1 or Compound 2 for 4 hours. Cells were harvested and mRNA expression analyzed by qPCR.

FIG. 3. Compound 2 induces chemokine and cytokine expression via ALPK1. ALPK1 knockout (KO) mice and wildtype (WT) control were treated orally with either PBS or Compound 2 (0.5 mg/kg). Four hours after treatment, livers were dissected for gene expression analysis by qPCR. Expression was normalized to PBS treated WT mice.

FIG. 4. Compound 2 activates cytokine expression only in the liver. 8 week old C57 females were administrated Compound 2 by oral gavage in 200 ul of Saline and 1.5% DMSO as diluent. Four hours later, organs were dissected and gene expression of CCL2 and CCL7 was analyzed by qPCR in kidney, esophagus, liver, lung, brain, and stomach.

FIG. 5A-B. Oral administration of HMP1BP derivatives activates chemokine and cytokine expression in liver cells. 8 week old C57 female mice were administered saline or the indicated compounds by oral gavage, Compounds 2-7 (1 mg·kg) (A) or compounds 9, 10 (1 mg/kg), 11, 13 and 14 (0.1 mg/kg) (B) were tested. Four hours after administration of saline or compound, organs were dissected and gene expression was analyzed by qPCR for CCL2, CCL7, CXCL1, CXCL10, IFNb, IL1b, IL6, and TNFa (A) or CCL2 and CCL7 (B).

FIG. 6A-C. Compound 2 reduces hepatitis infection in murine model. Compound 2 was administered (1 mg/kg PO QD) and serum levels of HBV (A), HbsAg (B), or HbeAg (C) were measured after 7 days.

### DETAILED DESCRIPTION

The disclosure provides compounds that are derivatives of certain bacterial metabolites in the ADP-heptose biosynthetic pathway, compositions comprising same, and methods for their use in therapy.

### Definitions

As used herein, the term “ALPK1” may refer to either one of two splice variants, isoform 1 or isoform 2, of the human ALPK1 gene. Each isoform shares the same kinase domain. For reference, the human ALPK1 gene is identified by Entrez Gene ID 80216.

As used herein, the term “activation of ALPK1” refers to the activation of ALPK1 kinase activity. In embodiments, the disclosure provides methods of activating ALPK1 by providing an ALPK1 agonist which may be, for example, an ALPK1 activating ligand, such as HBP, or a prodrug, analog or derivative thereof. Methods for making synthetic HBP are known, for example, as described in Inuki S et al. Organic Letter 2017 19(12):3079-82. In embodiments, the ALPK1 agonist is selected from HMP-lbP and H1b-ADP and prodrugs, analogs and derivatives thereof. In embodiments, the ALPK1 agonist is H1b-ADP, or a prodrug, analog or derivative thereof. In some embodiments, the disclosure provides methods of activating ALPK1 by providing an ALPK1 agonist represented by formula I, Ia, Ib, Ic, or Id.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. In some embodiments, alkyl groups are substituted with 1-2 substituents. As a non-limiting example, suitable substituents include halogen and hydroxyl.

As used herein, “alkenyl” refers to a straight chain or branched hydrocarbon having at least 2 carbon atoms and at least one double bond. Alkenyl can include any number of carbons, such as C2, C₂₋₃, C₂₋₄, C₂₋₅, C₂₋₆, C₂₋₇, C₂₋₈, C₂₋₉, C₂₋₁₀, C3, C₃₋₄, C₃₋₅, C₃₋₆, C₄, C₄₋₅, C₄₋₆, C₅, C_{5_6}, and C₆. Alkenyl groups can have any suitable number of double bonds, including, but not limited to, 1, 2, 3, 4, 5 or more. Alkenyl groups can be substituted or unsubstituted.

As used herein, the term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)n-, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted. In some embodiments, alkylene groups are substituted with 1-2 substituents. As a non-limiting example, suitable substituents include halogen and hydroxyl.

As used herein, the term “alkoxy” or “alkoxyl” refers to an alkyl group having an oxygen atom that connects the alkyl group to the point of attachment: alkyl-O—. As for alkyl group, alkoxyl groups can have any suitable number of carbon atoms, such as C1-6. Alkoxyl groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be substituted or unsubstituted.

As used herein, the term “alkenyloxy” or “alkenyloxyl” refers to an alkenyl group, as defined above, having an oxygen atom that connects the alkenyl group to the point of attachment: alkenyl-O—. Alkenyloxyl groups can have any suitable number of carbon atoms, such as C1-6. Alkenyloxyl groups can be further substituted with a variety of substituents described within. Alkenyloxyl groups can be substituted or unsubstituted.

As used herein, the term “alkylamine” or “alkylamino” refers to an alkyl group having a nitrogen atom that connects the alkyl group to the point of attachment: alkyl-N—. As for alkyl group, alkoxyl groups can have any suitable number of carbon atoms, such as C1-6.

As used herein, the term “halogen” refers to fluorine, chlorine, bromine and iodine.

As used herein, the term “haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc.

As used herein, the term “haloalkoxyl” or “haloalkoxy” refers to an alkoxyl group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens.

As used herein, the term “alkanoyl” refers to an alkyl group having a carbonyl group that connects the alkyl group to the point of attachment: alkyl-C(O)—. As for alkyl group, alkanoyloxyl groups can have any suitable number of carbon atoms, such as C1-4. For example, an alkanoyl groups include acetyl, propinoyl, butyryl, etc.

As used herein, the term “alkanoyloxyl” refers to an alkanoyl group having a an oxygen atom that connects the alkanoyl group to the point of attachment: alkyl-C(O)—O—. As for the alkyl group, alkanoyloxyl groups can have any suitable number of carbon atoms, such as C1-4. Exemplary alkanoyloxyl groups include acetoxy, propionyloxy, butryloxy, etc.

As used herein, the term “oxo” refers to an oxygen atom connected to the point of attachment by a double bond (═O).

As used herein, the term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. In some embodiments, aryl groups are substituted with 1-2 substituents. As a non-limiting example, suitable substituents include halogen, hydroxyl, —NO2, C1-8 alkyl, C1-8 alkoxy.

As used herein, the term “aralkyloxyl” refers to an aryl group, as defined above, having an alkyl and oxygen atom that connects the aryl group to the point of attachment: aryl-alkyl-O—. As for alkyl group, aralkyloxyl groups can have any suitable number of carbon atoms, such as C₁₋₄.

As used herein, the term “heteroaryl” refers to a monocyclic or fused bicyclic aromatic ring assembly containing 5 to 12 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 9 ring members and from 1 to 4 heteroatoms, or from 5 to 9 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), purine. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

As used herein, “cycloalkyl” refers to a saturated ring assembly containing from 3 to 8 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈. Cycloalkyl rings include, for example, cyclopropyl, cyclobutyl, cyclopentyl, cyclohexyl, and cyclooctyl. Cycloalkyl groups can be substituted or unsubstituted.

As used herein, “heterocyclyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. The N atom can further be substituted to form tertiary amine or ammonium salts. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, tetrahydrofuran, oxane (tetrahydropyran), oxepane, thiolane (tetrahydrothiophene), thiane (tetrahydrothiopyran), oxazolidine, isoxazolidine, thiazolidine, isothiazolidine, dioxolane, dithiolane, morpholine, etc. Heterocycloalkyl groups can be unsubstituted or substituted. For example, heterocycloalkyl groups can be substituted with C₁₋₆ alkyl or oxo (═O), among many others.

Certain compounds of the present invention possess asymmetric carbon atoms (optical centers) or double bonds; the racemates, diastereomer, geometric isomers, regioisomers and individual isomers (e.g., separate enantiomers) are all intended to be encompassed within the scope of the present invention. In some embodiments, the compounds of the present invention are a particular enantiomer, anomer, or diastereomer substantially free of other forms.

Certain compounds of the present disclosure include one or more thiophosphate moieties. The current disclosure generally displays the thiophosphate moiety as

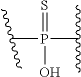 [SMILES: *P(*)(O)=S]

However, a person of skill in the art will recognize that the thiophosphate moiety can interconvert to

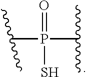 [SMILES: *P(*)(=O)S]

All stable interconversions of the thiophosphate moieties of the present disclosure are within the scope of this application.

As used herein, the term “substantially free” refers to an amount of 10% or less of another form, preferably 8%, 5%, 4%, 3%, 2%, 1%, 0.5%, or less of another form. In some embodiments, the isomer is a stereoisomer.

### DETAILED DESCRIPTION OF THE EMBODIMENTS

The present disclosure provides compounds represented by formula (I), or a stereoisomer, a stable isotope, prodrug or pharmaceutically acceptable salt thereof:

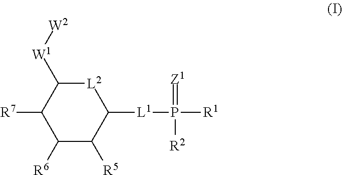

and/or a stereoisomer, tautomer, stable isotopes, prodrug or pharmaceutically acceptable salt thereof, wherein:
L¹ is selected from O, S, CH₂, CHF, CF2, OCH₂, SCH₂, OCHF, SCHF, OCF₂ or SCF₂;
L² is selected from the group consisting of O, S, CH₂, NR, CH₂, CH(OH), CHF and CF₂, wherein R is H or C1-C8 alkyl substituted with 0-3 substituents selected from halo, —OH, ═O, C1-C4 alkoxy, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members;
Z¹ is selected from O and S;
W¹ is —C(R¹⁰R¹¹)—, wherein R¹⁰ and R¹¹ are independently selected from H, D, —OH, halogen, and optionally substituted groups selected from C1-C4 alkyl, C1-C4 alkoxyl, C1-C4 haloalkyl, C1-C4-haloalkoxy, C1-C4 alkenyloxy, aralkyloxy, and 1-6 membered oligopeptidyl linked via C-termional C(O)O— and R¹²CO₂—, wherein R¹² is selected from C1-C20 alkyl, C1-C20 alkenyl, C1-C20 alkoxy, C1-C20 alkenyloxy, C1-C20 alkylamino, C3-C6 cycloalkyl, heterocyclyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members and 1-6 membered oligopeptidyl linked via N-terminal N; wherein the optional substituents for R¹⁰ and R¹¹ are 1-3 substituents independently selected from D, halogen, —OH, ═O, C1-C4 alkyl and C1-C4 alkoxy;
W² is R¹³-Q¹-W³—, wherein Q¹ is selected from —O— or —NH—; W³ is selected from a bond or C1-C3 alkylene groups optionally substituted with 1-3 substituents independently selected from halogen, —OH, ═O, C1-C3 alkoxy, C1-C3 haloalkyl, C1-C3 haloalkoxyl, C1-C3 alkenyloxy; wherein R¹³ is 1-6 membered oligopeptidyl linked via C-terminal carbonyl group or R¹⁴Q²C(O)—; wherein Q² is a bond, —O— or —NH—; R¹⁴ is 1-6 membered oligopeptidyl linked via N-terminal N or an optionally substituted group selected from C1-C20 alkyl, C1-C20 alkylenyl, C1-C20 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members, and R¹⁴ is R¹⁵-Q³-Q⁴-Q⁵-; wherein Q³, Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;
R¹ and R² are independently selected from the group consisting of —OR^{a}, and —NR^{b}R^{c}; when both R¹ and R² are —OR^{a}, the R^{a} moieties can combine to form a five or six-membered heterocyclic ring, wherein
   the five or six-membered heterocyclic ring is substituted with from 0 to 3 R³ moieties selected from the group consisting of H, D, halogen, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members, wherein the aryl or the 5 or 6 membered heteroaryl are substituted with 0 to 3 R^{3a} substituents selected from the group consisting of halogen and C1-C8 alkyl; or
   when two R³ substituents are on adjacent ring vertices of the five or six-membered heterocyclic ring, they can combine to form a fused phenyl ring, which is substituted with from 0 to 3 R⁴ moieties selected from the group consisting of H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R^{a} is selected from the group consisting of H, D, C1-C12 alkyl, C1-C12 haloalkyl, —C(R^{a1})(R^{a2})C(O)OR^{a3}, —C(R^{a1})(R^{a2})OC(O)R^{a3}, 3 to 6 membered heterocycloalkyl having 1-3 heteroatoms selected from N, O, and S as ring members, aryl, 5 to 10 membered heteroaryl, —C1-C4 alkylene-aryl, and —C1-C4 alkylene-5 to 10 membered heteroaryl,
   wherein the 5 or 10 membered heteroaryl has 1-3 heteroatoms selected from the group consisting of O, N, and S as ring members and the 5 or 10 membered heteroaryl is substituted with from 0 to 2 substituents selected from the group consisting of halogen, C1-C8 alkyl, and —NO₂.
each R^{b} and R^{c} are independently selected from the group consisting of H, C1-C12 alkyl, C1-C12 alkoxyl, C1-C12 alkanoyloxyl, C1-C12 alkenyloxyl, C3-C6 cycloalkyl, 4 to 6 membered heterocyclyoalkyl having 1-3 heteroatoms selected from N, O and S as ring members, aryl, 5 to 10 membered heteroaryl having 1-3 heteroatoms selected from N, O and S as ring members, and —C(R^{b1})(R^{b2})C(═O)OR^{b3};
each R^{a1}, R^{a2}, R^{b1}, and R^{b2} is selected from the group consisting of H, D, and C1-C4 alkyl C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocycloalkyl having 1-3 heteroatoms selected from N, O and S as ring members, aryl, and 5 to 10 membered heteroaryl having 1-3 heteroatoms selected from N, O and S as ring members;
each R^{a3} and R^{b3} is independently H, D, C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members; and
R⁵, R⁶ and R⁷ are independently selected from —OH, halogen, and R¹²CO₂—, and at least two of R⁵, R⁶ and R⁷ are —OH or R¹²CO₂, wherein R¹² is selected from C1-C8 alkyl, C1-C8 alkoxyl, C1-C8 alkanoyloxyl, C1-C8 alkenyloxyl, C1-C8 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members; wherein any two of the adjacent groups of R⁵, R⁶ and R⁷ can cyclize to form heterocycloalkyl containing 5 to 9 ring members and having 1-3 heteroatoms selected from N, O and S as ring members, each substituted by 0-3 substituents independently selected from D, CN, halogen, —OH, ═O, C1-C4 alkyl and C1-C4 alkoxy.

In some embodiments, the compound of formula I is a compound represented by Formula 1a

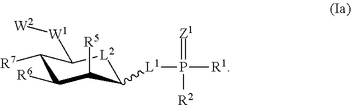

In some embodiments, the compound of formula I is a compound represented by Formula Ib

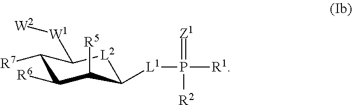

In some embodiments, the compound of formula I is a compound represented by Formula Ic

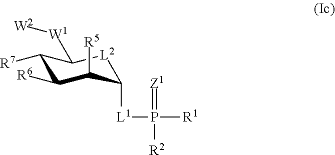

In some embodiments of the compounds of formulas Ia, Ib, and Ic, L² is selected from the group consisting of O, S, and CH₂; and optionally L¹ is selected from the group consisting of O, S, CH₂, CHF, and CF₂, or L¹ is O; and further optionally Z¹ is O.

In some embodiments of the compounds of formulas Ia, Ib, and Ic, L² is O; and optionally L¹ is selected from the group consisting of O, S, CH₂, CHF, and CF₂, or L¹ is O; and further optionally Z¹ is O.

In some embodiments of the compounds of formulas I, Ia, Ib, and Ic, R¹ and R² are each —OR^{a} and the R^{a} moieties can combine to form a five or six-membered heterocyclic ring, wherein
the five or six-membered heterocyclic ring is substituted with from 0 to 3 R³ moieties selected from the group consisting of H, D, halogen, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members, wherein the aryl or the 5 or 6 membered heteroaryl are substituted with 0 to 3 R^{3a} substituents selected from the group consisting of halogen and C₁-C₈ alkyl; or
   when two R³ substituents are on adjacent ring vertices of the five or six-membered heterocyclic ring, they can combine to form a fused phenyl ring, which is substituted with from 0 to 3 R⁴ moieties selected from the group consisting of H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members.

In some embodiments, the combined R^{a} moieties along with the oxygen and phosphorous atoms to which they are attached are represented by Formula iii,

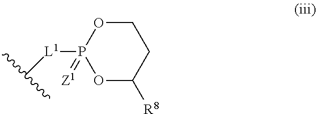

wherein R⁸ is selected from the group consisting of aryl, 3 to 6 membered heterocycloalkyl, and 5 or 6 membered heteroaryl wherein the 3 to 6 membered heterocycloalkyl and the 5 to 10 membered heteroaryl each have 1-3 heteroatoms selected from N, O and S as ring members, and the wavy line indicates the point of attachment to the rest of the molecule.

In some embodiments, the combined R^{a} moieties along with the oxygen and phosphorous atoms to which they are attached are represented by Formula ii,

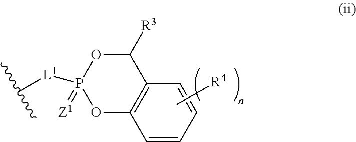

wherein R³ is selected from the group consisting of H, D, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R⁴ is independently selected from H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
the subscript n is an integer from 1 to 3; and the wavy line indicates the point of attachment to the rest of the molecule.

In some embodiments of the compounds of formulas I, Ia, Ib, and Ic, R¹ and R² are selected from the group consisting of —OR^{a}, —NR^{b}R^{c}.

In some embodiments of the compounds of formulas I, Ta, Tb, and Ic, where R¹ and R² are selected from the group consisting of —OR^{a}, —NR^{b}R^{c}, R¹ and R² combined with the phosphate to which they are attached are represented by Formula i

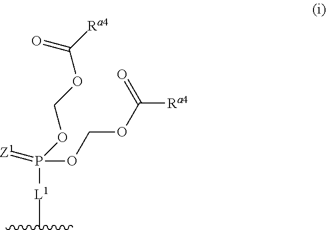

wherein each R^{a4} is each independently selected from C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members, and the wavy line indicates the point of attachment to the rest of the molecule.

In some embodiments of the compounds of formulas I, Ia, Ib, and Ic, where R¹ and R² are selected from the group consisting of —OR^{a}, —NR^{b}R^{c}, R¹ and R² combined with the phosphate to which they are attached are represented by Formula iv

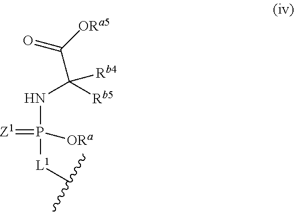

wherein R^{b4} and R^{b5} are optional independently H or D, C1-C4 alkyl, C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocyclyoalkyll having 1-3 heteroatoms selected from N, O and S as ring members, aryl, and 5 to 10 membered heteroaryl having 1-3 heteroatoms selected from N, O and S as ring members;
R^{a5} is H, D, C1-C12 alkyl, C1-C12 alkoxyl, C1-C12 alkanoyloxyl, C1-C12 alkenyloxyl, C1-C12 alkylamino, aralkyloxyl, C3-C6 cycloalkyl, 4 to 6 membered heterocyclyoalkyll having 1-3 heteroatoms selected from N, O and S as ring members, aryl, and 5 to 10 membered heteroaryl having 1-3 heteroatoms selected from N, O and S as ring members;
R^{a} is H, D, aryl or 3 to 6 ring membered heterocyclyoalkyl having 1-3 heteroatoms selected from N, O and S as ring members, —C1-C4 alkylene-aryl, and —C1-C4 alkylene-5 to 10 membered heteroaryl, wherein the 5 or 10 membered heteroaryl has 1-3 heteroatoms selected from the group consisting of O, N, and S as ring members; and the wavy line indicates the point of attachment to the rest of the molecule.

In some embodiments of the compounds of formulas I, Ia, Ib, and Ic, where R¹ and R² are selected from the group consisting of —OR^{a}, —NR^{b}R^{c}, R¹ and R² combined with the phosphate to which they are attached are represented by Formula v

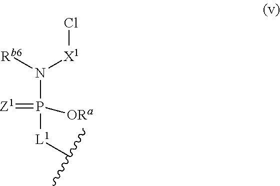

wherein R^{b6} is H, C1-C12 alkyl, C1-C12 alkoxyl, C1-C12 alkanoyloxyl, C1-C12 alkenyloxyl, C3-C6 cycloalkyl, 4 to 6 membered heterocycloalkyl having 1-3 heteroatoms selected from N, O and S as ring members, aryl, 5 to 10 membered heteroaryl having 1-3 heteroatoms selected from N, O and S as ring members;
X¹ is C₃₋₅ alkylene;
and R^{a} is H, D, 3 to 6 ring membered heterocyclyoalkyl having 1-3 heteroatoms selected from N, O and S as ring members, aryl, and 5 to 10 membered heteroaryl, —C1-C4 alkylene-aryl, and —C1-C4 alkylene-5 to 10 membered heteroaryl, wherein the 5 or 10 membered heteroaryl has 1-3 heteroatoms selected from the group consisting of O, N, and S as ring members; and the wavy line indicates the point of attachment to the rest of the molecule.

In some embodiments of the compounds of formulas I, Ta, Tb, and Ic, where R¹ and R² are selected from the group consisting of —OR^{a}, —NR^{b}R^{c}, the compound is represented by Formula Id

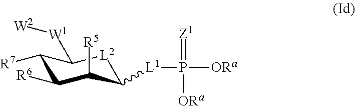

wherein each R^{a} is phenyl.

In some embodiments of the compounds of formulas I, Ia, Ib, Ic and Id where R⁵, R⁶ and R⁷ are independently selected from —OH, halogen, and R¹²CO₂—, at least two of R⁵, R⁶ and R⁷ are —OH or R¹²CO₂.

In some embodiments, the compound of formulas I, Ia, Ib, Ic, or Id is a compound selected from Table 1, or a stereoisomer, a stable isotope, prodrug or a pharmaceutically acceptable salt thereof.

In some embodiments, the compound of formula I is a compound described in the Examples of this application.

The disclosure also provides pharmaceutical compositions comprising a compound of formulas I, Ia, Ib, Ic, and Id.

The compounds of the present disclosure can be prepared using the general processes describes in Schemes I, II, and III as well as the techniques described in the exemplary embodiments.

In embodiments, the disclosure provides an ALPK1 agonist in the form of a compound of formulas I, Ia, Ib, Ic, and Id described herein, and prodrugs, analogs and derivatives thereof.

In embodiments, the disclosure provides methods of treating cancer by administering a compound of formulas I, Ia, Ib, Ic, and Id described herein, and prodrugs, analogs and derivatives thereof. In further embodiments of the methods of treating cancer, the disclosure provides a combination therapy comprising administering a compound of formulas I, Ia, Ib, Ic, and Id described herein, and prodrugs, analogs and derivatives thereof, in combination with an immune checkpoint modulator selected from a checkpoint inhibitor, such as an anti-PD-1/PD-L1 antibody, and an agonist of an immune co-stimulatory molecule, such as an anti-OX40 (CD134) agonist antibody. Without being bound by any specific theory, the inventors propose that H1b-ADP and its derivatives described herein may promote the antigen-presenting functions of tumor infiltrating antigen presenting cells (APC) and tumor-specific T cell proliferation and differentiation. In addition, these molecules may also heighten the recruitment of tumor-specific CD8+ T cells to tumors by increasing PD-L1 expression in tumor cells.

In embodiments, the disclosure provides methods of modulating an immune response in a subject, the methods comprising administering to the subject a composition comprising a compound of formulas I, Ia, Ib, Ic, and Id described herein, and prodrugs, analogs and derivatives thereof.

In embodiments, the disclosure provides methods of potentiating an immune response to a target antigen in a subject, the methods comprising administering to the subject a composition comprising a compound of formulas I, Ia, Ib, Ic, and Id described herein, and prodrugs, analogs and derivatives thereof. In embodiments, the target antigen may be an antigen of an infectious agent, such as a bacterial antigen, a viral antigen, or an antigen of a parasite. In embodiments, the antigen is a tumor antigen. In accordance with any of these embodiments, a compound of formulas I, Ia, Ib, Ic, and Id described herein, and prodrugs, analogs and derivatives thereof, may serve as an adjuvant to a vaccine composition for the treatment or prevention of a disease or disorder caused by an infectious agent, or for the treatment of cancer, or for the treatment of another disease or disorder that may be treated with a vaccine composition, including, for example, Alzheimer's disease. In embodiments, the antigen is selected from amyloid protein in the treatment of Alzheimer's disease. In embodiments, the antigen is selected from glycoprotein 100 (gp100), mucin 1 (MUC1), and melanoma-associated antigen 3 (MAGEA3) in the treatment of cancer. In embodiments, the cancer is selected from breast, ovarian, or prostate cancer. In embodiments, the cancer is HTLV-1 T-lymphotropic leukemia.

In embodiments, the cancer is melanoma and a compound of formulas I, Ia, Ib, Ic, and Id described herein, and prodrugs, analogs and derivatives thereof, may serve as an adjuvant to treatment with Talimogene laherparepvec (T-VEC), or may be used in a combination therapy regimen with T-VEC.

In embodiments for the treatment or prevention of an infectious disease, a compound of formulas I, Ia, Ib, Ic, and Id described herein, and prodrugs, analogs and derivatives thereof, may serve as an adjuvant to a vaccine composition for the treatment or prevention of anthrax, caries, Chagas disease, dengue, diphtheria, ehrlichiosis, hepatitis A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, and visceral leishmaniasis.

In embodiments for the treatment or prevention of an infectious disease, the a compound of formulas I, Ia, Ib, Ic, and Id described herein, and prodrugs, analogs and derivatives thereof, may serve as an adjuvant to a vaccine composition for the treatment or prevention of a disease or disorder caused by adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), rotavirus, Salmonella typhi, Staphylococcus aureus, Streptococcus pyogenes, varicella, West Nile virus, Yersinia pestis, and Zika virus.

In accordance with any of the foregoing embodiments, the method may comprise administering a vaccine composition or adjuvant comprising a compound of formulas I, Ia, Ib, Ic, and Id described herein, and prodrugs, analogs and derivatives thereof.

In embodiments, the disclosure provides methods of treating a disease or disorder amendable to treatment by activation of NFkB, p38, and JNK cell signaling pathways in cells of a subject, the method comprising administering to the subject a compound of formulas I, Ia, Ib, Ic, and Id described herein, and prodrugs, analogs and derivatives thereof. In embodiments, the disease or disorder is caused by a bacterial, viral, or parasitic infection, as described in more detail below, and including for example diseases and disorders caused by the hepatitis C virus (HCV), the hepatitis B virus (HBV), and the human immunodeficiency virus (HIV). In embodiments, the disease or disorder is selected from tuberculosis, meningitis, pneumonia, ulcer, and sepsis. In embodiments, the disease or disorder is selected from rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis and bullous diseases. In embodiments, the disease or disorder is selected from actinic keratoses, ulcerative colitis, Crohn's disease, and alopecia areata.

In embodiments, the disclosure provides methods of treating or preventing a bacterial, viral, or parasitic infection in a subject in need thereof, the methods comprising administering to the subject a composition comprising a compound of formulas I, Ia, Ib, Ic, and Id described herein, and prodrugs, analogs and derivatives thereof.

In embodiments, the method is a method of treating or preventing a bacterial infection. In embodiments, the bacterial infection is caused by a Gram-negative or a Gram-positive bacteria. In embodiments, the bacteria is a Gram-negative bacteria selected from the group consisting of Acinetobacter baumanii, Aggregatobacter actinomycetemcomitans, Bartonella bacilliformis, Bartonella henselae, Bartonella quintana, Bifidobacterium Borrelia, Bortadella pertussis, Brucella sp, Burkholderia cepacis, Burkholderia pseudomallei, Campylobacter jejuni, Cardiobacterium hominis, Campylobacter fetus, Chlamydia pneumonia, Chlymydia trachomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucinobilis, Pseudomonas putida, Pseudomonas fluorescens, Pseudomonas acidovorans, Rickettsiae, Salmonella enterica, Salmonella typhi, Salmonella paratyphi types A, B. typhus, Salmonella dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus and Yersinia pestitis.

In embodiments, the bacteria is a Gram-positive bacteria selected from the group consisting of Actinomycetes, Bacillus anthracis, Bacillus subtilis, Clostridium tetani, Clostridium perfingens, Clostridium botulinum, Clostridium tetani, Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, and Streptococcus mutants.

In embodiments, the method is a method of treating or preventing a viral infection. In embodiments, the viral infection is caused by a virus selected from the group consisting of Adeno-associated virus, Aichi virus, Alpha virus, Arena virus, Arobovirus, Australian bat lyssavirus, BK polyomavirus, Banna virus, Birnavirus, Bornavirus, bunyamwera virus, Bunyavirus La Crosse, Bunyavirus snowshoe hare, Valicivirus, Cercopithecine herpesvirus, Chandipura virus, Chikugunya virus, Cosavirus A, Coxpox virus, Coxsakievirus, Crimean-Congo hemorrhagic fever virus, Dengue virus, Dhori virus, Dugbe virus, Devenhage virus, Eastern equine encephalitis virus, Ebolavirus, Echovirus, Encephalomyocarditis virus, Epstein-Barr virus, European bat lyssavirus, Flavivirus, GB virus/Hepatitis G virus, Hantaan virus, Hendra virus, hepadnavirus, Hepatitis A virus, Hepatitis B virus, Hepatitis C virus, Hepatitis E virus, Hepatitis delta virus, Herpes simplex virus, horsepox virus, human adenovirus, human astrovirus, human coronavirus, human cytomegalovirus, human enterovirus 68,70, human herpesvirus 1, human herpesvirus 2, human herpesvirus 6, human herpesvirus 7, human herpesvirus 8, human immunodeficiency virus (HIV), human papillomavirus (HPV-6, HPV-11), human spumaretrovirus, human T-lymphotropic virus, human torovirus, Influenza A virus, Influenza B virus, Influenza C virus, Isfaha virus, JC polyomavirus, Japanese encephalitis virus, Junin arenavirus, Kaposi's sarcoma (HHV-8), KI polyomavirus, Kunjin virus, Lagos bat virus, Lake Vitoria marbugvirus, Langat virus, Lassa virus, LMC virus, Lordsdale virus, Louping ill virus, Lymphocytic choriomeningitis virus, Machupovirus, Marmath forest virus, Mayaro virus, MERS coronavirus, Measles virus, Mengo encephalomycarditis virus, Merkel cell polyomavirus, mlluscum contagiosum, parvovirus B19, Mokola virus, Mumps virus, Murray valley encephalitis virus, New York virus, Nipha virus, Norwalk virus, O'nyong-hyong virus, Orf virus, Oropouche virus, Orthomyxovirus, parainfluenza virus, paramyxovaris, parvovirus, Phchinde virus, picomavirus, poliovirus, polyomavirus, poxvirus, Punta toro phleboviris, Puumala virus, rabdovirus, Rabies virus, reovirus, rhinovirus, respiratory syncytial virus, Rift valley fever virus, Rosavirus A, Ross river virus, Rotavirus A, Rotavirus B, Rotavirus C, Rubella virus, Sagiyama virus, Salivirus A, Sandfly fever sicillian virus, Sapporo virus, Semliki forest virus, Seoul virus, Simian foamy virus, Simian virus 5, Sindbis virus, Southampton virus, St. louis encephalitis virus, Tick-borne powassan virus, togavirus, Torque virus, Toscana virus, Uukuniemi virus, Vaccina virus, Varicella-zoster virus, Variola virus, Venezuelan equine encephalitis virus, Vesicular stomatitits virus, Western equine encephalitis virus, UU polyomavirus, West Nile virus, Yaba monkey tumor virus, Yaba-like disease virus, Yellow fever virus, and Zika virus.

In embodiments, the method is a method of treating or preventing a parasitic infection. In embodiments, the parasitic infection is caused by parasite selected from the group consisting of Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In embodiments, the disclosure provides methods of treating cancer in a subject, the methods comprising administering to the subject a composition comprising a compound of formulas I, Ia, Ib, Ic, and Id described herein, and prodrugs, analogs and derivatives thereof. In embodiments, the cancer is selected from soft tissue sarcoma, breast cancer, head and neck cancer, melanoma, cervical cancer, bladder cancer, hematologic malignancy, glioblastoma, pancreatic cancer, prostate cancer, colon cancer, breast cancer, renal cancer, lung cancer, merkel cell carcinoma, small intestine cancer, thyroid cancer, acute myelogenous leukemia (AML), acute lymphocytic leukemia (ALL), chronic lymphocytic leukemia (CLL), chronic myelogenous leukemia (CML), gastric cancer, gastrointestinal stromal tumors, non-Hodgkins lymphoma, Hodgkins lymphoma, liver cancer, leukemia, lymphoma, T-cell lymphoma.

In embodiments of any of the methods described here the compound of formulas I, Ia, Ib, Ic, and Id described herein, and prodrugs, analogs and derivatives thereof, may be administered in combination with one or more additional therapeutic agents or immune modulators, including for example in combination with a vaccine or vaccine adjuvant. In embodiments, the one or more additional therapeutic agents is an inhibitor or antagonist of, or a vaccine against, an immune checkpoint molecule including, for example, the programed cell death 1 (PD-1) receptor (CD279), a ligand of PD-1 (e.g., PD-L1), cytotoxic T-lymphocyte associated protein 4 (CTLA4), tumor necrosis factor receptor superfamily member 9 (alternatively TNFRSF9, 4-1BB) and 4-1BB ligands, tumor necrosis factor receptor superfamily member 4 (alternatively TNFRSF4, OX40) and OX40 ligands, glucocorticoid-induced TNFR-related protein (GITR), Tumor Necrosis Factor Receptor Superfamily Member 7 (alternatively TNFRSF7, cluster of differentiation 27, CD27), TNFRSF25 and TNF-like ligand 1A (TL1A), TNF Receptor Superfamily Member 5 (alternatively TNFRSF5, CD40) and CD40 ligand, Herpesvirus entry mediator (HVEM)-tumor necrosis factor ligand superfamily member 14 (alternatively TNFSF14, LIGHT)-lymphotoxin alpha (LTA), herpesvirus entry mediator-(HVEM)-B- and T-lymphocyte attenuator (BTLA)-CD160 (alternatively TNFSF14), lymphocyte activating gene 3 (LAG3), T-cell immunoglobulin and mucin-domain containing-3 (TIM3), sialic-acid-binding immunoglobulin-like lectins (SIGLECs), inducible T-cell costimulator (ICOS) and ICOS ligand, B7-H3 (B7 family, alternatively CD276), V-set domain-containing T-cell activation inhibitor 1 (VTCN1, alternatively B7-H4), V-Type immunoglobulin domain-containing suppressor T-ell activation VISTA), human endogenous retrovirus-H long terminal repeat-associating protein 2 (HHLA2)-transmembrane and Immunoglobulin domain containing 2 (TMIGD2), butyrophilins, natural killer cell receptor 2B4 (alternatively NKR2B4, CD244) and B-Cell Membrane Protein (CD48), T-Cell Immunoreceptor with Immunoglobulin (Ig) and immunoreceptor tyrosine-based inhibition motif domains (TIGIT) and Poliovirus receptor (PVR) family members, killer-cell immunoglobulin-like receptors (KIRs), Immunoglobulin-like transcripts (ILTs) and leukocyte immunoglobulin-like receptor (LIRs), natural killer group protein 2 member D (NKG2D) and natural killer group protein 2 member A (NKG2A), major histocompatibility complex (MHC) class I polypeptide-related sequence A (MICA) and MHC class I polypeptide-related sequence B (MICB), natural killer cell receptor 2B4 (CD244), colony stimulating factor 1 receptor (CSF1R), indoleamine 2,3-dioxygenase (IDO), transforming growth factor beta (TGFβ), Adenosine-ecto-nucleotidase triphosphate diphosphohydrolase 1 (CD39)-5′-nucleotidase (CD73), C—X—C motif chemokine receptor 4 (CXCR4) and C—X—C motif chemokine ligand 12 (CXCL12), phosphatidylserine, signal regulatory protein alpha (SIRPA) and integrin associated protein (CD47), vascular endothelial growth factor (VEGF), and neuropilin.

In embodiments of any of the methods described here the compound of formulas I, Ia, Ib, Ic, and Id described herein, and prodrugs, analogs and derivatives thereof, may be administered in combination with a checkpoint inhibitor or an agonist of an immune co-stimulatory molecule, such as an anti-OX40 (CD134) agonist antibody. In embodiments, the checkpoint inhibitor is a PD-1/PD-L1 inhibitor, such as an anti-PD1 antibody or an anti-PD-L1 antibody, and the ALPK1 agonist is selected from Hib-ADP-6L and H1b-ADP, and prodrugs, analogs and derivatives thereof.

In embodiments, a compound of formulas I, Ia, Ib, Ic, and Id described herein, and prodrugs, analogs and derivatives thereof, may be administered in combination with one or more immune modulators. In embodiments, the immune modulator may be a vaccine. In embodiments, the vaccine is a vaccine against an infectious agent, as described above. In embodiments, the vaccine is a cancer vaccine. In embodiments, the cancer vaccine targets a tumor antigen selected from glycoprotein 100 (gp100), mucin 1 (MUC1), and melanoma-associated antigen 3 (MAGEA3).

In embodiments, the one or more immune modulators may be a recombinant protein, for example, granulocyte-macrophage colony-stimulating factor (GM-CSF), interleukin 7 (IL-7), IL-12, IL-15, IL-18, or IL-21.

In embodiments of the treatment of cancer, a compound of formulas I, Ia, Ib, Ic, and Id described herein, and prodrugs, analogs and derivatives thereof, may be administered in combination with a T cell therapy, such as chimeric antigen receptor (CAR) T cell therapy,

In embodiments of the methods for treating cancer a compound of formulas I, Ta, Tb, Ic, and Id described herein, and prodrugs, analogs and derivatives thereof, may be administered in combination with a PD-1/PD-L1 inhibitor or an agonist of an immune co-stimulatory molecule, such as an anti-OX40 (CD134) agonist antibody. In embodiments, the cancer is selected from advanced melanoma, non-small cell lung cancer, renal cell carcinoma, bladder cancer, liver cancer, gastric cancer, colon cancer, breast cancer, non-Hodgkin's lymphoma, prostate cancer, head and neck cancer, thyroid cancer, brain cancer, acute myeloid leukemia (AML), merkel cell carcinoma, multiple myeloma, cervical cancer, and sarcoma and the method further comprises administering a PD-1/PD-L1 inhibitor or an agonist of an immune co-stimulatory molecule to the subject.

In embodiments of the methods for modulating an immune response or for treating or preventing a bacterial, viral, or parasitic infection, the one or more additional therapeutic agents may be an immune modulator, for example, an inhibitor or antagonist of immune checkpoint molecule. Such molecules generally act as key regulators of the immune system, for example, as co-stimulators of the immune response.

In embodiments, the disclosure also provides a vaccine composition or vaccine adjuvant comprising a compound of formulas I, Ia, Ib, Ic, and Id described herein, and prodrugs, analogs and derivatives thereof. A vaccine composition described here may further comprise one or more adjuvants.

In embodiments, the disclosure also provides a pharmaceutical composition comprising a compound of formulas I, Ia, Ib, Ic, and Id described herein, and prodrugs, analogs and derivatives thereof.

In the context of the methods described here, the term “treating” may refer to the amelioration or stabilization of one or more symptoms associated with the disease, disorder or condition being treated. The term “treating” may also encompass the management of disease, disorder or condition, referring to the beneficial effects that a subject derives from a therapy but which does not result in a cure of the underlying disease, disorder, or condition. In the context of the present disclosure, the term “prevention” refers to preventing the recurrence, development, progression or onset of one or more symptoms of the disease, disorder, or condition.

In embodiments where a therapeutically effective amount of a compound or composition is administered to a subject, the therapeutically effective amount is the amount sufficient to achieve a desired therapeutic outcome, for example the amelioration or stabilization of one or more symptoms of the disease, disorder or condition being treated, or in the context of prevention, the amount sufficient to achieve prevention of the recurrence, development, progression or onset of one or more symptoms of the disease, disorder, or condition.

In embodiments, a therapeutically effective amount is the amount required to achieve at least an equivalent therapeutic effect compared to a standard therapy. An example of a standard therapy is an FDA-approved drug indicated for treating the same disease, disorder or condition.

In the context of any of the methods described here, the subject is preferably a human but may be a non-human vertebrate. In other embodiments, the non-human vertebrate may be, for example, a dog, cat, a rodent (e.g., a mouse, a rat, a rabbit), a horse, a cow, a sheep, a goat, a chicken, a duck, or any other non-human vertebrate.

In embodiments, the human subject is selected from an adult human, a pediatric human, or a geriatric human, as those terms are understood by the medical practitioner, for example as defined by the U.S. Food and Drug Administration.

In embodiments, the disclosure provides a composition comprising an ALPK1 agonist, or a composition comprising a polynucleotide encoding ALPK1, or a composition comprising ALPK1 protein, and one or more excipients or carriers, preferably pharmaceutically acceptable excipients or carriers. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Excipients for preparing a pharmaceutical composition are generally those that are known to be safe and non-toxic when administered to a human or animal body. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, and suitable mixtures of any of the foregoing. The particular excipients utilized in a composition will depend upon various factors, including chemical stability and solubility of the compound being formulated and the intended route of administration.

A pharmaceutical composition can be provided in bulk or unit dosage form. It is especially advantageous to formulate pharmaceutical compositions in unit dosage form for ease of administration and uniformity of dosage. The term “unit dosage form” refers to physically discrete units suited as unitary dosages for the subject to be treated; each unit containing a predetermined quantity of an active compound calculated to produce the desired therapeutic effect in association with the required pharmaceutical carrier. A unit dosage form can be an ampoule, a vial, a suppository, a dragee, a tablet, a capsule, an IV bag, or a single pump on an aerosol inhaler.

In therapeutic applications, dose may vary depending on the chemical and physical properties of the active compound as well as clinical characteristics of the subject, including e.g., age, weight, and co-morbidities. Generally, the dose should be a therapeutically effective amount. An effective amount of a pharmaceutical composition is that which provides an objectively identifiable improvement as noted by the clinician or other qualified observer. For example, alleviating a symptom of a disorder, disease or condition.

A pharmaceutical compositions may take any suitable form (e.g. liquids, aerosols, solutions, inhalants, mists, sprays; or solids, powders, ointments, pastes, creams, lotions, gels, patches and the like) for administration by any desired route (e.g. pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). In embodiments, the pharmaceutical composition is in the form of an orally acceptable dosage form including, but not limited to, capsules, tablets, buccal forms, troches, lozenges, and oral liquids in the form of emulsions, aqueous suspensions, dispersions or solutions. Capsules may contain excipients such as inert fillers and/or diluents including starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added.

In embodiments, the pharmaceutical composition is in the form of a tablet. The tablet can comprise a unit dose of a compound described here together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. butylated hydroxytoluene), buffering agents (e.g. phosphate or citrate buffers), and effervescent agents such as citrate/bicarbonate mixtures. The tablet may be a coated tablet. The coating can be a protective film coating (e.g. a wax or varnish) or a coating designed to control the release of the active compound, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine.

In embodiments, the pharmaceutical composition is in the form of a hard or soft gelatin capsule. In accordance with this formulation, the compound of the present invention may be in a solid, semi-solid, or liquid form.

In embodiments, the pharmaceutical composition is in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

In embodiments, the pharmaceutical composition is in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions can be prepared in water with the aid of co-solvent or a surfactant. Examples of suitable surfactants include polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides. Dispersions can also be prepared, for example, in glycerol, liquid polyethylene glycols and mixtures of the same in oils.

In embodiments, a compound or composition described here may be administered as monotherapy or adjunctive therapy. In embodiments, a compound or composition described here may be administered alone or in combination with one or more additional therapeutic agents (i.e., additional APIs) or therapies, for example as part of a therapeutic regimen that includes, e.g., aspects of diet and exercise). In embodiments, the methods described here include administration of a compound of formulas I, Ia, Ib, Ic, and Id described herein, and prodrugs, analogs and derivatives thereof, as the primary therapy. In other embodiments, the administration of a compound of formulas I, Ia, Ib, Ic, and Id described herein, and prodrugs, analogs and derivatives thereof, is an adjuvant therapy. In either case, the methods of the invention contemplate the administration of a compound of formulas I, Ia, Ib, Ic, and Id described herein, and prodrugs, analogs and derivatives thereof, in combination with one or more additional therapeutic agents and/or therapies for the treatment or prevention of a disease, disorder, or condition as described here. The terms “therapy” and “therapies” refer to any method, protocol and/or agent that can be used in the prevention, treatment, management or amelioration of a disease, disorder, or condition, one or more symptoms thereof.

The present disclosure also provides packaging and kits comprising pharmaceutical compositions for use in the methods described here. The kit can comprise one or more containers selected from the group consisting of a bottle, a vial, an ampoule, a blister pack, and a syringe. The kit can further include one or more of instructions for use, one or more syringes, one or more applicators, or a sterile solution suitable for reconstituting a compound or composition described here.

### Preparation of Compounds of Formula I and Exemplary Compounds

### Type I: Carbonyloxymethyl

Carbonyloxymethyl is a class of phosphate protecting groups. In some embodiments, carbonyloxymethyl protecting groups have the generic Formula i

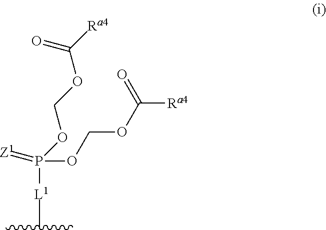

wherein each R^{a4} is each independently selected from C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members, and the wavy line indicates the point of attachment to the rest of the molecule. In some embodiments, each R^{a4} is independently C₁₋₈ alkyl or C₁₋₈ alkoxy.

Without being bound to any particular theory, it is believed that phosphate groups protected by carbonyloxymethyl moieties are deprotected in vivo through a series of chemical conversions described in Scheme I, below.

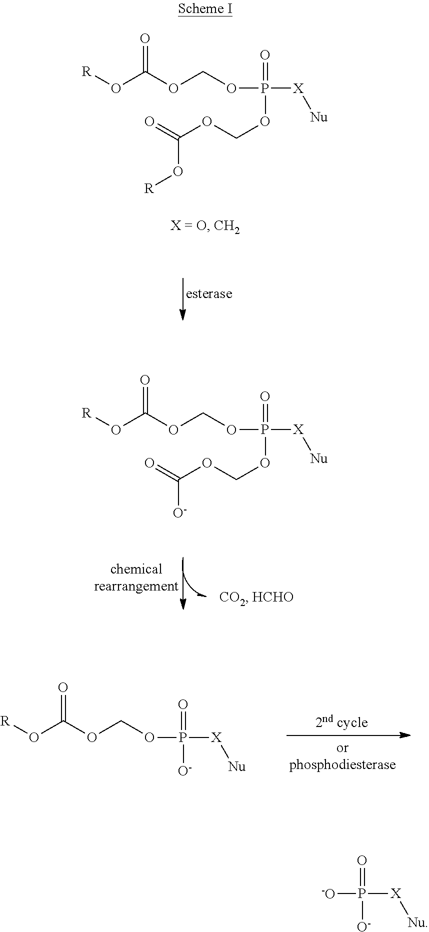

In some embodiments, the prodrug of HMP has a Formula (Ia-1a) and (Ia-1b)

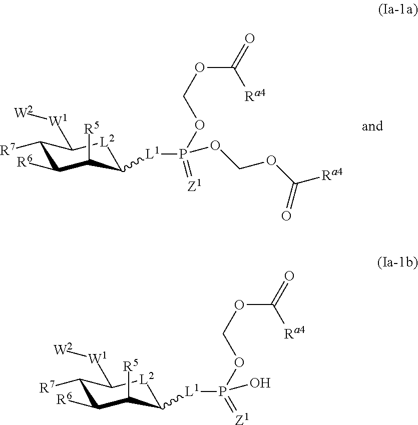

wherein each R^{a4} is independently C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl containing having 1-3 heteroatoms selected from N, O and S as ring members, aryl, and 5 to 10 membered heteroaryl having 1-3 heteroatoms selected from N, O and S as ring members. In some embodiments, each R^{a4} is independently C₁₋₈ alkyl or C₁₋₈ alkoxy.

Carbonyloxymethyl prodrugs of HMP can be prepared using the methods described in Hwang, Y. and Cole, P. A. Organic Letters 2004, 6, 1555.

Type II: Cyclosaligenyl (cycloSal)

Cyclosaligenyl (cycloSal) are a class of phosphate protecting groups. In some embodiments, cycloSal protecting groups have the generic Formula ii

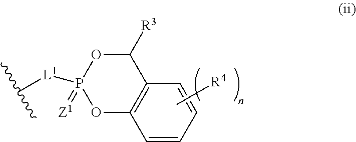

wherein R³ is selected from the group consisting of H, D, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R⁴ is independently selected from H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
the subscript n is an integer from 1 to 3; and
the wavy line indicates the point of attachment to the rest of the molecule.

In some embodiments, R³ is H or C₁₋₈ alkyl. In some embodiments, R⁴ is C₁₋₈ alkyl. In some embodiments, the subscript n is 1.

Without being bound to any particular theory, it is believed that phosphate groups protected by one or more cycloSal moieties are deprotected in vivo via the pathways described in Scheme II, below.

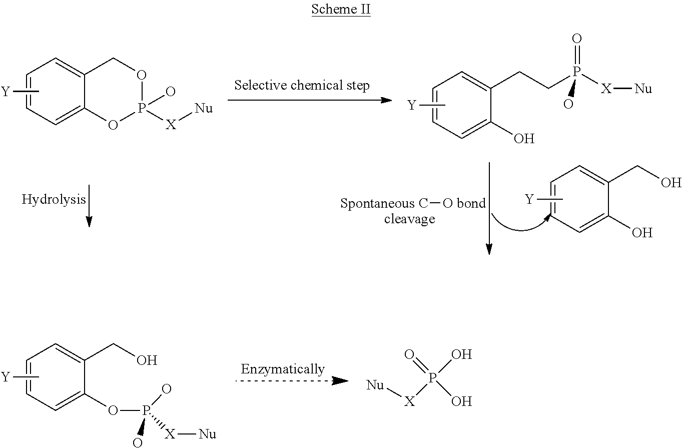

In some embodiments, the prodrug of HMP has a Formula Ia-2

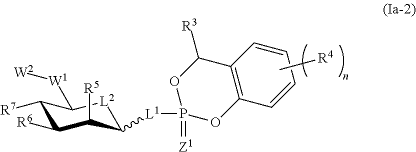

wherein R³ is selected from the group consisting of H, D, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R⁴ is independently selected from H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
the subscript n is an integer from 1 to 3; and
the wavy line indicates the point of attachment to the rest of the molecule

In some embodiments, R³ is H or C1-8 alkyl. In some embodiments, R⁴ is C1-8 alkyl. In some embodiments, the subscript n is 1.

CycloSal prodrugs of HMP can be prepared using the methods described in Spáčilová, P. et al., Chem.Med.Chem 2010, 5, 1386.

Type III: Cyclic 1-aryl-1,3-propanyl Ester (HepDirect)

Cyclic 1-aryl-1,3-propanyl esters (HepDirects) are a class of phosphate protecting groups. In some embodiments, HepDirect protecting groups have the generic Formula iii

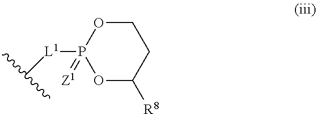

wherein R⁸ is aryl or 3 to 6 membered heterocycloalkyl and 5 to 10 membered heteroaryl, wherein the 3 to 6 membered heterocycloalkyl and the 5 to 10 membered heteroaryl each have 1-3 heteroatoms selected from N, O and S as ring members, and the wavy line indicates the point of attachment to the rest of the molecule. In some embodiments, R⁸ is aryl or 6-membered heteroaryl. In some embodiments R⁸ is phenyl or pyridyl.

Without being bound to any particular theory, it is believed that phosphate groups protected by HepDirect moieties are deprotected in vivo via the pathway described in Scheme III, below.

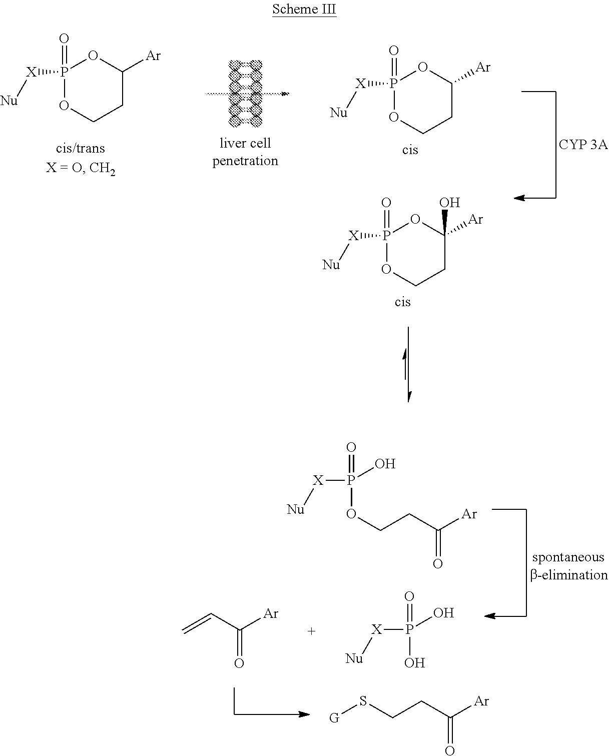

In some embodiments, the prodrug of HMP has a Formula Ia-3

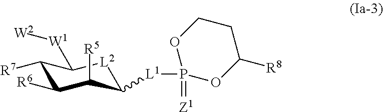

wherein R⁸ is aryl or 3 to 6 membered heterocycloalkyl and 5 to 10 membered heteroaryl, wherein the 3 to 6 membered heterocycloalkyl and the 5 to 10 membered heteroaryl each have 1-3 heteroatoms selected from N, O and S as ring members, and the wavy line indicates the point of attachment to the rest of the molecule. In some embodiments, R⁸ is aryl or 6-membered heteroaryl. In some embodiments R⁸ is phenyl or pyridyl.

HepDirect prodrugs of HMP can be prepared using the methods described in Reddy, K. R. et al., Tetrahedron Letters 2005, 46, 4321.

### Type IV: Aryloxy Amino Acid Amidate (Protide)

Aryloxy amino acid amidates (Protides) are a class of phosphate protecting group. In some embodiments protide protecting groups have the generic Formula iv

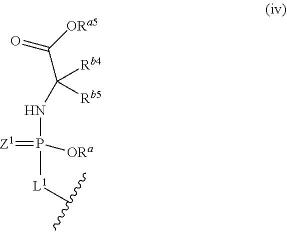

wherein R^{b4} and R^{b5} are optional independently H or D, C1-C4 alkyl, C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocyclyoalkyll having 1-3 heteroatoms selected from N, O and S as ring members, aryl, and 5 to 10 membered heteroaryl having 1-3 heteroatoms selected from N, O and S as ring members;

R^{a5} is H, D, C1-C12 alkyl, C1-C12 alkoxyl, C1-C12 alkanoyloxyl, C1-C12 alkenyloxyl, C1-C12 alkylamino, aralkyloxyl, C3-C6 cycloalkyl, 4 to 6 membered heterocyclyoalkyll having 1-3 heteroatoms selected from N, O and S as ring members, aryl, and 5 to 10 membered heteroaryl having 1-3 heteroatoms selected from N, O and S as ring members;

R^{a} is H, D, aryl or 3 to 6 ring membered heterocyclyoalkyll having 1-3 heteroatoms selected from N, O and S as ring members, —C1-C4 alkylene-aryl, and —C1-C4 alkylene-5 to 10 membered heteroaryl, wherein the 5 or 10 membered heteroaryl has 1-3 heteroatoms selected from the group consisting of O, N, and S as ring members; and the wavy line indicates the point of attachment to the rest of the molecule. In some embodiments, R^{a5} is methyl or isopropyl. In some embodiments, R^{a} is phenyl.

Without being bound to any particular theory, it is believed that phosphate groups protected by Protide moieties are de-protected in vivo via the pathway described in Scheme IV, below.

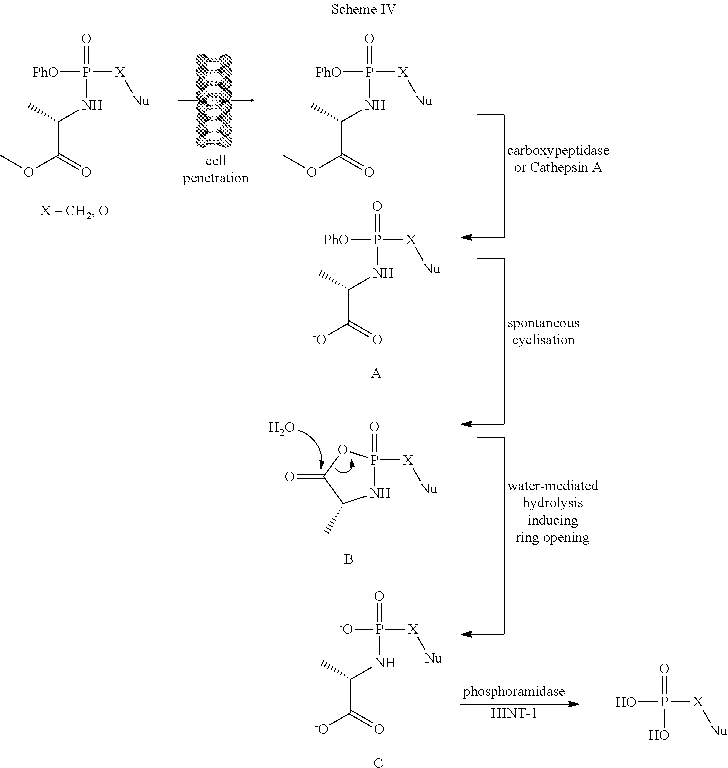

In some embodiments, the prodrug of HMP has a Formula Ia-4a or Ia-4b

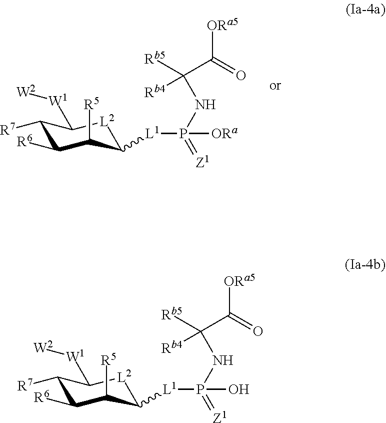

wherein R^{b4} and R^{b5} are optionally independently H or D, C1-C4 alkyl, C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocyclyoalkyll having 1-3 heteroatoms selected from N, O and S as ring members, aryl, and 5 to 10 membered heteroaryl having 1-3 heteroatoms selected from N, O and S as ring members;

R^{a5} is H, D, C1-C12 alkyl, C1-C12 alkoxyl, C1-C12 alkanoyloxyl, C1-C12 alkenyloxyl, C1-C12 alkylamino, aralkyloxyl, C3-C6 cycloalkyl, 4 to 6 membered heterocyclyoalkyll having 1-3 heteroatoms selected from N, O and S as ring members, aryl, and 5 to 10 membered heteroaryl having 1-3 heteroatoms selected from N, O and S as ring members;

R^{a} is H, D, aryl or 3 to 6 membered heterocyclyoalkyll containing 3 to 6 ring members and having 1-3 heteroatoms selected from N, O and S as ring members, aryl, and heteroaryl containing 5 to 10 ring atoms and having 1-3 heteroatoms selected from N, O and S as ring members; and the wavy line indicates the point of attachment to the rest of the molecule.

In some embodiments, R^{7a} is methyl or isopropyl. In some embodiments, R^{8a} is phenyl.

Protide prodrugs of HMP can be prepared using the methods described in van Boom, J. H. et al., Tetrahedron 1975, 31, 2953.

### Type V: Methylaryl Haloalkylamidate

Methylaryl haloalkylamidates are a class of phosphate protecting groups. In some embodiments methylaryl haloalkylamdiate protecting groups have the generic Formula v

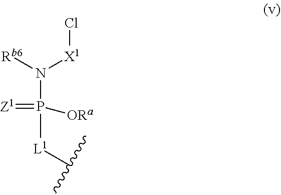

wherein R^{b6} is H, C1-C12 alkyl, C1-C12 alkoxyl, C1-C12 alkanoyloxyl, C1-C12 alkenyloxyl, C3-C6 cycloalkyl, 4 to 6 membered heterocyclyoalkyl having 1-3 heteroatoms selected from N, O and S as ring members, aryl, and 5 to 10 membered heteroaryl having 1-3 heteroatoms selected from N, O and S as ring members; X¹ is C₃₋₅ alkylene; and

R^{a} is H, D, 3 to 6 ring membered heterocycloalkyl having 1-3 heteroatoms selected from N, O and S as ring members, aryl, 5 to 10 membered heteroaryl, —C1-C4 alkylene-aryl, and —C1-C4 alkylene-5 to 10 membered heteroaryl, wherein the 5 or 10 membered heteroaryl has 1-3 heteroatoms selected from N, O and S as ring members. In some embodiments, R^{b6} is C₁₋₄ alkyl. And the wavy line indicates the point of attachment to the rest of the molecule. In some embodiments, X¹ is C₄ alkylene. In some embodiments, R^{a} is aryl or aryl C₁₋₄alkylene. In some embodiments, R^{a} is phenyl. In some embodiments, R^{a} is benzyl.

Without being bound to any particular theory it is believed that phosphate groups protected by methylaryl haloalkylamdiate moieties are deprotected in vivo through a series of chemical conversion described in Scheme V below. It is understood that the groups defined for R⁹ and R¹⁰ are exemplary and are not intended to be limiting.

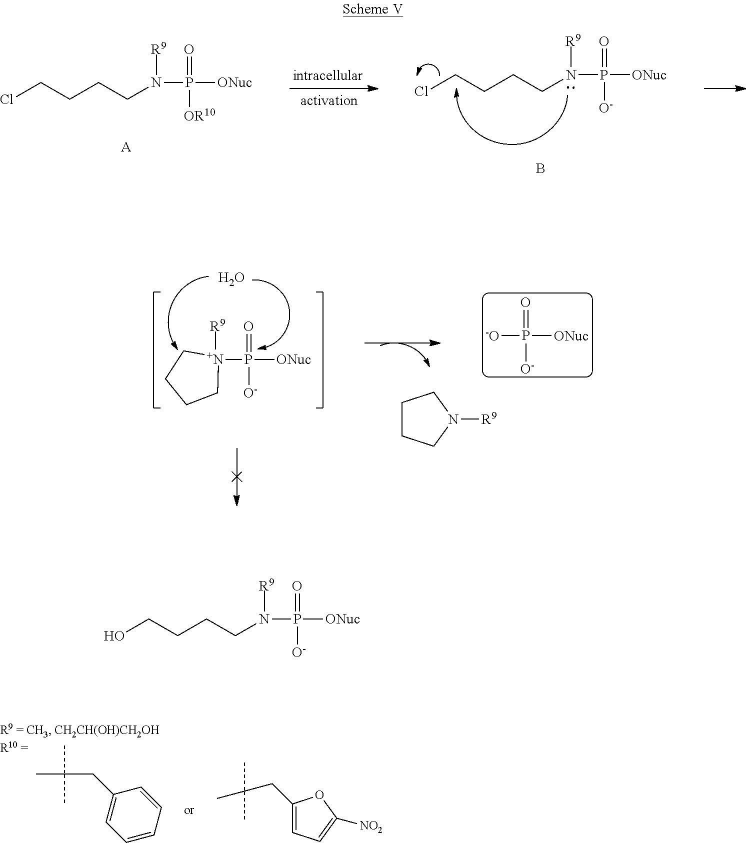

In some embodiments, the prodrug of HMP has a Formula Ia-5a or Ia-5b

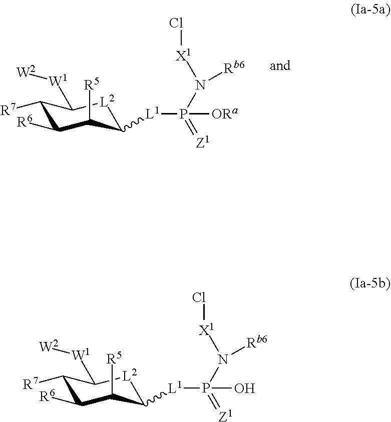

wherein R^{b6} is H, C1-C12 alkyl, C1-C12 alkoxyl, C1-C12 alkanoyloxyl, C1-C12 alkenyloxyl, C3-C6 cycloalkyl, 4 to 6 membered heterocyclyoalkyl having 1-3 heteroatoms selected from N, O and S as ring members, aryl, and 5 to 10 membered heteroaryl having 1-3 heteroatoms selected from N, and S as ring members;

X¹ is C₃₋₅ alkylene; and

R^{a} is H, D, 3 to 6 membered heterocyclyoalkyl having 1-3 heteroatoms selected from N, O and S as ring members, aryl, 5 to 10 membered heteroaryl, —C1-C4 alkylene-aryl, and —C1-C4 alkylene-5 to 10 membered heteroaryl, wherein the 5 or 10 membered heteroaryl has 1-3 heteroatoms selected from N, O and S as ring members. In some embodiments, R^{b6} is C₁₋₄ alkyl. And the wavy line indicates the point of attachment to the rest of the molecule. In some embodiments, X¹ is C₄ alkylene. In some embodiments, R^{a} is aryl or aryl C₁₋₄alkylene. In some embodiments, R^{a} is phenyl. In some embodiments, R^{a} is benzyl.

Methylaryl haloalkylamdiate prodrugs of HMP can be prepared using the methods described in Wu, W. et al., Journal of Medicinal Chemistry 2007, 50, 3743.

### Type VI: Esters

In some embodiments, the prodrug of HMP has a Formula Id

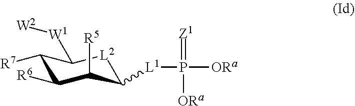

Each R^{a} is independently selected from H, D, C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members; In some embodiments, each R^{a} is independently C6 aryl with or without substitutions. In some embodiments, each R^{a} is independently phenyl.

The possible mechanism in vivo is described in scheme VI:

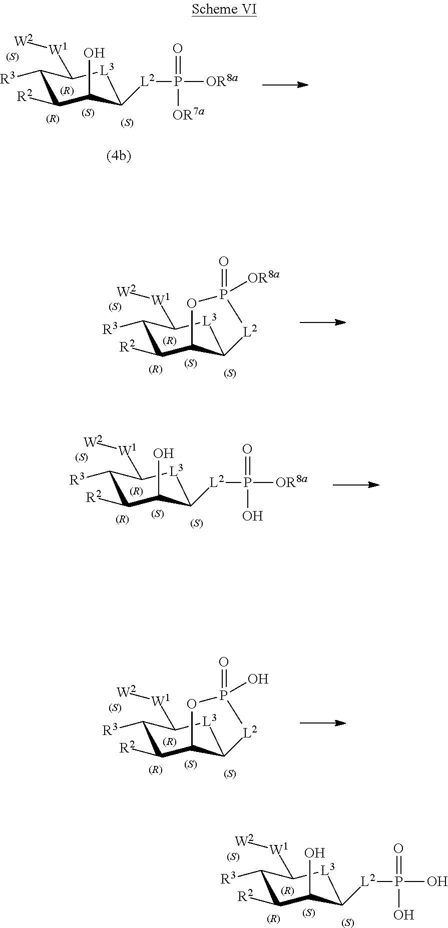

TABLE 1
Compounds of Formula I

| Compound | Chemical Name |
|---|---|
| 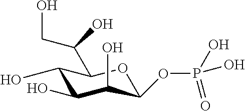 Compound 1 Reference HMP1BP | (2S,3S,4S,5S,6R)-6-((R)-1,2- dihydroxyethyl)-3,4,5- trihydroxytetrahydro-2H-pyran-2-yl dihydrogen phosphate |
| 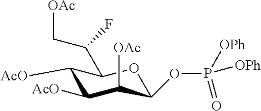 Compound 2 | (2S,3S,4S,5S,6S)-2-((S)-2-acetoxy-1- fluoroethyl)-6- ((diphenoxyphosphoryl)oxy)tetrahydro- 2H-pyran-3,4,5-triyl triacetate |
| 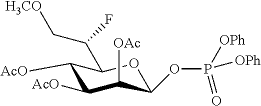 Compound 3 | (2S,3R,4S,5S)-2- ((diphenoxyphosphoryl)oxy)-6-((S)-1- fluoro-2-methoxyethyl)tetrahydro-2H- pyran-3,4,5-triyl triacetate |
| 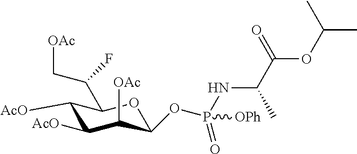 Compound 4 | (2S,3S,4S,5S,6S)-2-((S)-2-acetoxy-1- fluoroethyl)-6-(((((S)-1-isopropoxy-1- oxopropan-2- yl)amino)(phenoxy)phosphoryl)oxy)tetra- hydro-2H-pyran-3,4,5-triyl triacetate |
| 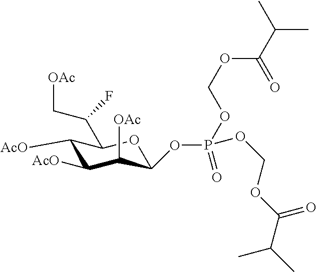 Compound 5 | (2S,3S,4S,5S,6S)-2-((S)-2-acetoxy-1- fluoroethyl)-6- ((bis((isobutyryloxy)methoxy)phosphoryl)oxy)tetra- hydro-2H-pyran-3,4,5-triyl triacetate |
| 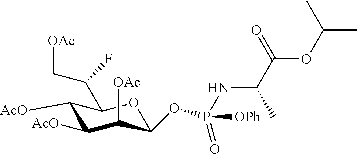 Compound 6 | (2S,3S,4S,5S,6S)-2-((S)-2-acetoxy-1- fluoroethyl)-6-(((R)-(((S)-1-isopropoxy-1- oxopropan-2- yl)amino)(phenoxy)phosphoryl)oxy)tetrahydro- 2H-pyran-3,4,5-triyl triacetate |
| 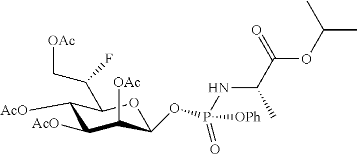 Compound 7 | (2S,3S,4S,5S,6S)-2-((S)-2-acetoxy-1- fluoroethyl)-6-(((S)-(((S)-1-isopropoxy-1- oxopropan-2- yl)amino)(phenoxy)phosphoryl)oxy)tetrahydro- 2H-pyran-3,4,5-triyl triacetate |
| 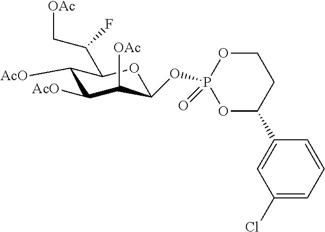 Compound 8 | (2S,3S,4S,5S,6S)-2-((S)-2-acetoxy-1- fluoroethyl)-6-(((2R,4R)-4-(3- chlorophenyl)-2-oxido-1,3,2- dioxaphosphinan-2-yl)oxy)tetrahydro-2H- pyran-3,4,5-triyl triacetate |
| 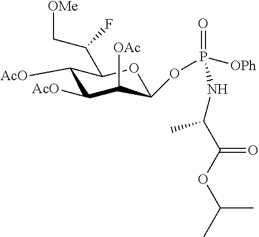 Compound 9 | Beta isomer of (2S,3S,4S,5S,6S)-2-((S)-1- fluoro-2-methoxyethyl)-6-(((S)-(((S)-1- isopropoxy-1-oxopropan-2- yl)amino)(phenoxy)phosphoryl)oxy)tetrahydro- 2H-pyran-3,4,5-triyl triacetate |
| 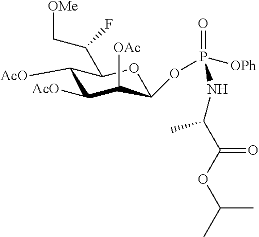 Compound 10 | Beta isomer of (2S,3S,4S,5S)-2-((S)-1- fluoro-2-methoxyethyl)-6-(((((S)-1- isopropoxy-1-oxopropan-2- yl)amino)(phenoxy)phosphoryl)oxy)tetrahydro- 2H-pyran-3,4,5-triyl triacetate |
| 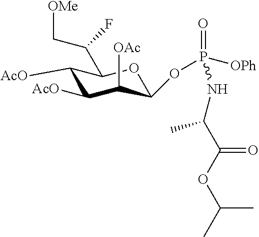 Compound 11 | Alfa isomer of (2S,3S,4S,5S)-2-((S)-1- fluoro-2-methoxyethyl)-6-(((((S)-1- isopropoxy-1-oxopropan-2- yl)amino)(phenoxy)phosphoryl)oxy)tetrahydro- 2H-pyran-3,4,5-triyl triacetate |
| 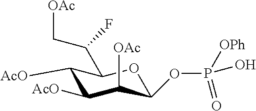 Compound 12 | (2S,3S,4S,5S,6S)-2-((S)-2-acetoxy-1- fluoroethyl)-6- ((hydroxy(phenoxy)phosphoryl)oxy)tetrahydro- 2H-pyran-3,4,5-triyl triacetate |
| 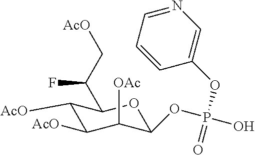 [SMILES: CC(=O)OC[C@H](F)C1OC(O[P@](=O)(O)Oc2cccnc2)C(OC(C)=O)C(OC(C)=O)C1OC(C)=O] Compound 13 | (2S,3S,4S,5S,6S)-2-((S)-2-acetoxy-1- fluoroethyl)-6-(((R)-hydroxy(pyridin-3- yloxy)phosphoryl)oxy)tetrahydro-2H- pyran-3,4,5-triyl triacetate |
| 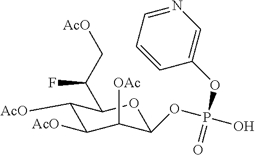 [SMILES: CC(=O)OC[C@H](F)C1OC(O[P@@](=O)(O)Oc2cccnc2)C(OC(C)=O)C(OC(C)=O)C1OC(C)=O] Compound 14 | (2S,3S,4S,5S,6S)-2-((S)-2-acetoxy-1- fluoroethyl)-6-(((S)-hydroxy(pyridin-3- yloxy)phosphoryl)oxy)tetrahydro-2H- pyran-3,4,5-triyl triacetate |
| 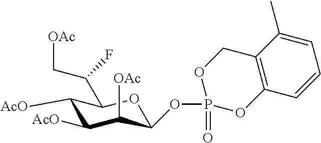 Compound 15 | (2S,3S,4S,5S,6S)-2-((S)-2-acetoxy-1- fluoroethyl)-6-((5-methyl-2-oxido-4H- benzo[d][1,3,2]dioxaphosphinin-2- yl)oxy)tetrahydro-2H-pyran-3,4,5-triyl triacetate |
| 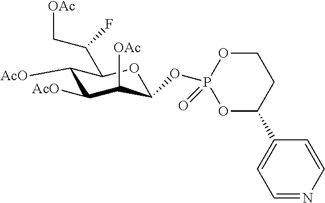 Compound 16 | (2S,3S,4S,5S,6S)-2-((S)-2-acetoxy-1- fluoroethyl)-6-(((4S)-2-oxido-4-(pyridin- 4-yl)-1,3,2-dioxaphosphinan-2- yl)oxy)tetrahydro-2H-pyran-3,4,5-triyl triacetate |
| 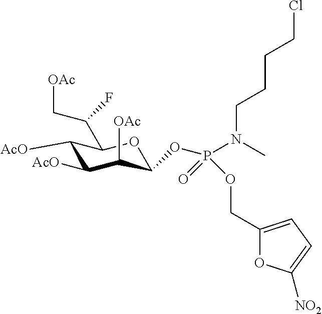 Compound 17 | (2S,3S,4S,5S,6S)-2-((S)-2-acetoxy-1- fluoroethyl)-6-((((4- chlorobutyl)(methyl)amino)((5- nitrofuran-2- yl)methoxy)phosphoryl)oxy)tetrahydro- 2H-pyran-3,4,5-triyl triacetate |
| 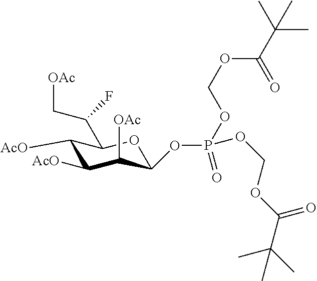 Compound 18 | (2S,3S,4S,5S,6S)-2-((S)-2-acetoxy-1- fluoroethyl)-6- ((bis((pivaloyloxy)methoxy)phosphoryl)oxy)tetra- hydro-2H-pyran-3,4,5-triyl triacetate |

### General Synthesis Scheme for Compounds of Formula I in Beta Configuration (Ib) of Phosphate Part:

The compounds of formula I in which L² is O can be made by general synthetic method as illustrated in Scheme 1. The substituted phosphorodichloridate is mixed with H—R⁵ in suitable solvent like DCM. Hunig's base is dropwise to this solution at −50˜−60° C. The mixture is stirred for 1-3 hours. Afterwards the mixture is warmed to 10-25° C. and stirred for further 2-4 hours to give compound II. The solution of compound II in suitable solvent like DCM is added dropwise to the solution of compound III and DMAP in the same solvent at 10-20° C. The reaction was stirred at 0-20° C. for 4-24 hours to give the compound IV.

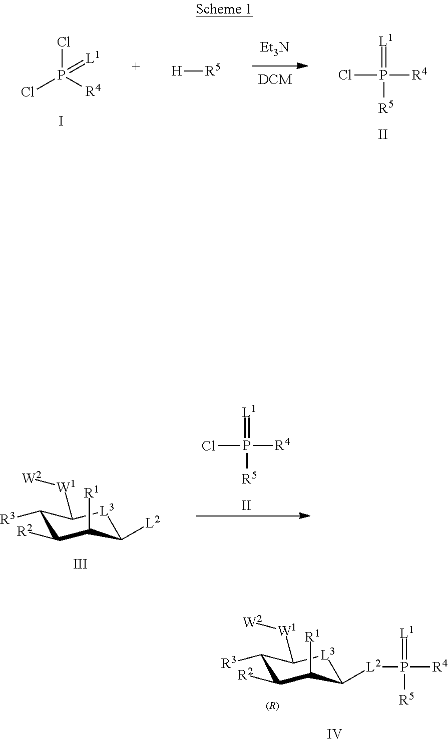

### General Synthesis Scheme for Compounds of Formula I in Alfa Configuration of Phosphate Part:

The compounds of formula I in which L² is O can be made by general synthetic method as illustrated in Scheme 2. The mixture of compound III in suitable solvent like THF under inert gas is treated with t-BuMgCl at −20° C. to 0° C. The mixture is stirred at such temperature for 0.5-2 hours. Compound V is added and the mixture is stirred at 10-20° C. under inert gas for 10-30 hours to give the compound VI.

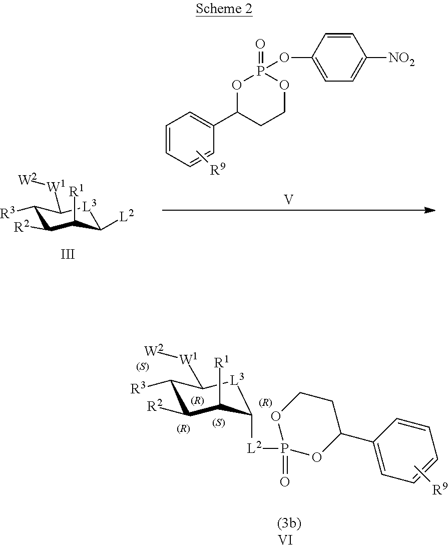

### General Synthesis Scheme for Compounds of Formula I

The compounds of formula I in which L² is O can be made by general synthetic method as illustrated in Scheme 3. To a solution of compound III and compound VII in suitable solvent like CH₃CN is added Ag₂CO₃. The mixture is stirred at 70° C. for 12 hours under inert gas to give compound VIII.

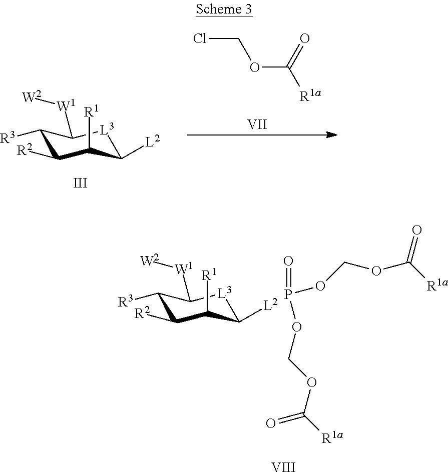

### Synthesis of Representative Compounds of Formula (I):

All moisture-sensitive reactions were performed using syringe-septum cap techniques under Ar. Analytical thin layer chromatography (TLC) was performed on Silica gel 60 F 254 Plates (Qindao, 0.25 mm thickness). ₁H-NMR spectra were recorded with a Varian-400 spectrometer, and chemical shifts were reported as (ppm) values relative to internal tetramethylsilane or the residual proton of the deuterated solvent. ₁₃C-NMR spectra were recorded with a Varian-400 spectrometer, and chemical shifts were reported as δ (ppm) values relative to internal tetramethylsilane or the residual proton of the deuterated solvent. ₃₁P-NMR spectra were recorded with a Varian-400 spectrometer, and chemical shifts were reported as δ (ppm) values relative to external 85% phosphoric acid. ₁H-NMR spectra are tabulated as follows: chemical shift, multiplicity (br=broad, s=singlet, d=doublet, t=triplet, q=quartet, m=multiplet), number of protons, and coupling constant(s). The Alfa and Beta conformation can be determined by 2D NMR. The enantiomers can be separated by Chiral HPLC and the presentation of chemistry structure of these enantiomers is arbitrary.

### Synthesis of Compound 2

### Step 1. Preparation of Compound (2S,3S,4S,5S,6S)-3,4,5-tris(benzyloxy)-2-((S)-1-fluoro-2-(trityloxy)ethyl)-6-methoxytetrahydro-2H-pyran

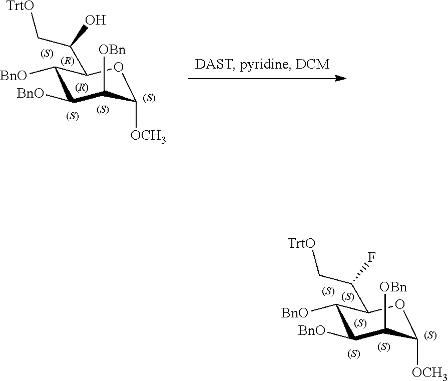

To the mixture of the compound (R)-1-((2R,3S,4S,5S,6S)-3,4,5-tris(benzyloxy)-6-methoxytetrahydro-2H-pyran-2-yl)-2-(trityloxy)ethan-1-ol (9.5 g, 12.9 mmol) (Tiehai Li et al., (2014) Bioorg. Med. Chem. 22: 1139-1147; Shinsuke Inuki et al., Org. Lett. (2017), 19: 3079-3082) in DCM (100 mL) were added DAST (10.4 g, 64.5 mmol, 8.5 mL) and pyridine (10.2 g, 128.9 mmol, 10.4 mL) at 0° C. The mixture was stirred at 25° C. for 16 h. The reaction was quenched with sat. NaHCO₃ (100 mL) carefully. The mixture was extracted with DCM (100 mL×3). The combined organic layers were washed with 2N HCl (150 mL), dried over Na₂SO₄ and concentrated under vacuum. The residue was purified by flash column chromatography (silica gel, PE:EA=1:0 to 12:1). The desired compound (4.2 g, Yield: 44.1%) was obtained as a light yellow oil. ¹H NMR (400 MHz, CDCl₃): δ 7.38-7.18 (m, 30H), 4.92-4.61 (m, 2H), 4.53-4.51 (m, 6H), 4.06-4.02 (m, 1H), 3.77-3.75 (m, 1H), 3.65-3.51 (m, 3H), 3.14-3.06 (m, 1H), 2.96 (s, 3H).

### Step 2. Preparation of Compound (S)-2-fluoro-2-((2S,3S,4S,5S,6S)-3,4,5-tris(benzyloxy)-6-methoxytetrahydro-2H-pyran-2-yl)ethan-1-ol

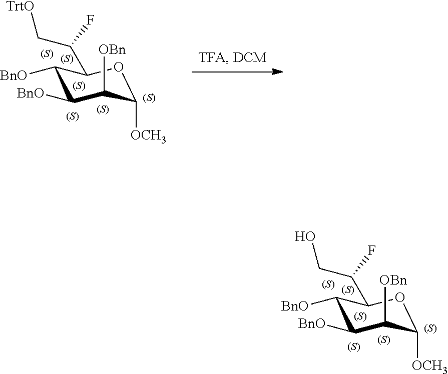

To the solution of the compound obtained from step 1 above (5.8 g, 7.9 mmol) in DCM (60 mL) was added TFA (13.9 g, 121.6 mmol, 9 mL). The mixture was stirred at 25° C. for 1 h. To the mixture was added sat. NaHCO₃ (150 mL). The mixture was extracted with DCM (100 mL×3). The combined organic layers were dried over Na₂SO₄ and concentrated under vacuum. The residue was purified by flash column chromatography (silica gel, PE:EA=10:1 to 1:1). The desired compound (3.2 g, Yield: 79.7%, 96.2% purity) was obtained as a colorless oil.

MS (ESI) m/z (M+H)⁺: 519.1. ¹H NMR (400 MHz, CDCl₃): δ 7.35-7.28 (m, 15H), 4.99-4.96 (m, 2H), 4.73-4.65 (m, 4H), 4.60 (s, 2H), 4.14-4.10 (m, 3H), 3.77-3.76 (m, 1H), 3.70 (m, 1H), 3.60-3.57 (m, 1H), 3.27 (s, 3H). ¹⁹F NMR δ-207.84.

### Step 3. Preparation of Compound (3S,4S,5S,6S)-6-((S)-2-acetoxy-1-fluoroethyl)-3,4,5-tris(benzyloxy)tetrahydro-2H-pyran-2-yl acetate

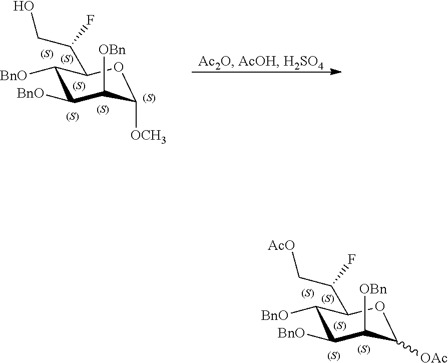

To the solution of the compound obtained from step 2 above (3.2 g, 6.5 mmol) in HOAc (15 mL) and Ac₂O (15 mL) was added H₂SO₄ (2.8 g, 27.6 mmol, 1.5 mL, 98% purity). The mixture was stirred at 25° C. for 1 h. The reaction was quenched with methanol (15 mL) at 0° C. Most of the solvent was removed under vacuum. 30 mL of sat. NaHCO₃ was added and the mixture was extracted with ethyl acetate (50 mL×3). The combined organic layers were washed with brine (50 mL), dried over Na₂SO₄ and concentrated under vacuum. The desired compound (3.9 g, crude) was obtained as a light yellow oil which was used for next step directly.

### Step 4. Preparation of Compound (3S,4S,5S,6S)-6-((S)-2-acetoxy-1-fluoroethyl)-3,4,5-trihydroxytetrahydro-2H-pyran-2-yl acetate

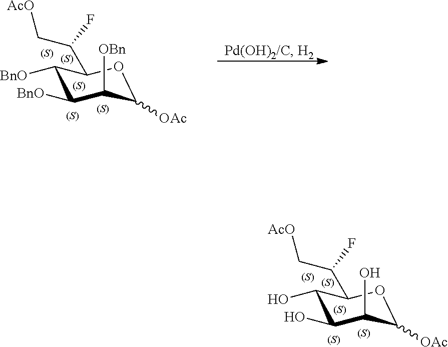

To the mixture of the product of Step 3 above (3.9 g, 6.9 mmol) in methanol (20 mL), THF (10 mL), H₂O (2 mL) and HOAc (0.5 mL) were added Pd(OH)₂/C (0.6 g, 20% purity) at 25° C. The mixture was stirred at 25° C. under hydrogen (50 psi) for 32 h. The mixture was filtered through celite and washed with methanol (50 mL×3). The filtrate was collected and concentrated under vacuum. The desired compound (2.5 g, crude) was obtained as a light yellow oil which was used for next step directly.

### Step 5. Preparation of Compound (3S,4S,5S,6S)-6-((S)-2-acetoxy-1-fluoroethyl)tetrahydro-2H-pyran-2,3,4,5-tetrayl Tetraacetate

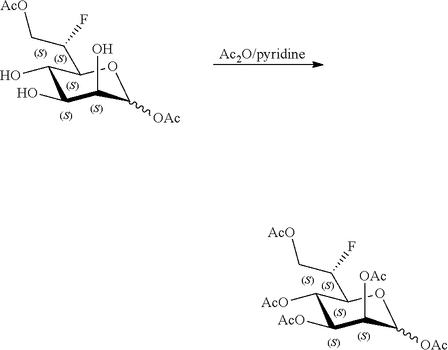

To the solution of the product of Step 4 above (2.5 g, 8.4 mmol) in pyridine (20 mL) were added Ac₂O (4.3 g, 42.2 mmol, 4.0 mL) and DMAP (515.5 mg, 4.2 mmol). The mixture was stirred at 25° C. for 0.5 h. The reaction was quenched with methanol (15 mL). Most of pyridine was removed under vacuum. 1N HCl (20 mL) was added to the residue. The residue was extracted with ethyl acetate (30 mL×3). The combined organic layers were washed with 2N HCl (30 mL), dried over Na₂SO₄ and concentrated under vacuum. The residue was purified by flash column chromatography (silica gel, PE:EA=10:1 to 3:2). The desired compound (1.6 g, Yield: 44.6%) was obtained as a colorless oil. MS (ESI) m/z (M+H)⁺: 445.0. ¹H NMR (400 MHz, CDCl₃): δ 6.07 (s, 1H), 5.54-5.49 (m, 1H), 5.34-5.31 (m, 1H), 5.24-5.22 (m, 1H), 4.70-4.56 (m, 1H), 4.38-4.24 (m, 2H), 3.98-3.89 (m, 1H), 2.16 (d, J=6.4 Hz, 6H), 2.06 (d, J=6.0 Hz, 6H), 1.99 (s, 3H).

### Step 6. Preparation of Compound (2S,3S,4S,5S)-2-((S)-2-acetoxy-1-fluoroethyl)-6-hydroxytetrahydro-2H-pyran-3,4,5-triyl triacetate

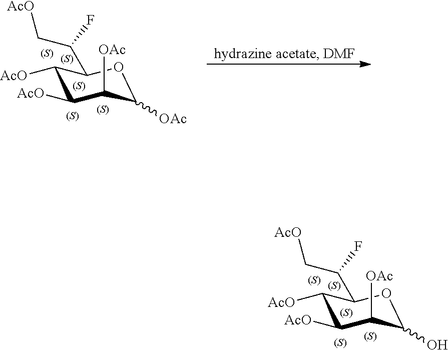

To the solution of the product of Step 5 (1.6 g, 3.8 mmol) in DMF (15 mL) was added hydrazine acetate (520.1 mg, 5.7 mmol). The mixture was stirred at 25° C. for 20 min. The reaction was quenched with H₂O (15 mL). The mixture was extracted with ethyl acetate (20 mL×3). The combined organic layers were washed with H₂O (20 mL×3), dried over Na₂SO₄ and concentrated under vacuum. The residue was purified by flash column chromatography (silica gel, PE:EA=10:1 to 1:1). The desired compound (860 mg, Yield: 60.1%) was obtained as a colorless oil.

¹H NMR (400 MHz, CDCl₃): δ 5.52-5.47 (m, 1H), 5.42-5.39 (m, 1H), 5.26-5.25 (m, 2H), 4.75-4.60 (m, 1H), 4.39-4.31 (m, 2H), 4.14-4.05 (m, 1H), 2.15 (s, 3H), 2.10 (s, 3H), 2.06 (s, 3H), 1.99 (s, 3H).

### Step 7. Preparation of Compound (2S,3S,4S,5S,6S)-2-((S)-2-acetoxy-1-fluoroethyl)-6-((diphenoxyphosphoryl)oxy)tetrahydro-2H-pyran-3,4,5-triyl triacetate

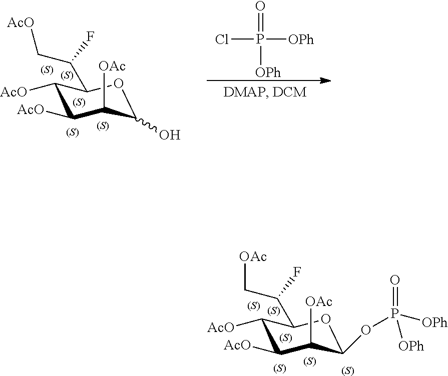

[chloro(phenoxy)phosphoryl]oxybenzene (2.1 g, 7.7 mmol, 1.6 mL) in DCM (50 mL) was added dropwisely to the solution of the compound obtained from Step 6 above (970 mg, 2.6 mmol) and DMAP (1.6 g, 12.8 mmol) in DCM (50 mL) at 25° C. within 3.5 h. The mixture was stirred at 25° C. for 16 h. The reaction was quenched with sat.NaHCO₃ (50 mL). The mixture was extracted with DCM (80 mL×3). The combined organic layers were dried over Na₂SO₄ and concentrated under vacuum. The residue was purified by flash column chromatography (silica gel, PE:EA=10:1 to 3:2). The desired compound (1.21 g, Yield: 77.5%, 100% purity) was obtained as a colorless oil. MS (ESI) m/z (M+H)⁺: 658.1. ¹H NMR (400 MHz, CDCl₃) δ 7.35-7.13 (m, 10H), 5.54 (d, J=6.8 Hz, 1H), 5.50-5.46 (m, 2H), 5.07-5.04 (m, 1H), 4.72-4.57 (m, 1H), 4.30-4.26 (m, 1H), 4.23-4.19 (m, 1H), 3.74-3.65 (m, 1H), 2.10 (s, 3H), 2.07 (s, 3H), 2.05 (s, 3H), 1.98 (s, 3H). ¹⁹F NMR δ-205.5.

### Synthesis of Compound 3

### Step 1. Preparation of Compound (2S,3S,4S,5S,6S)-3,4,5-tris(benzyloxy)-2-((S)-1-fluoro-2-methoxyethyl)-6-methoxytetrahydro-2H-pyran

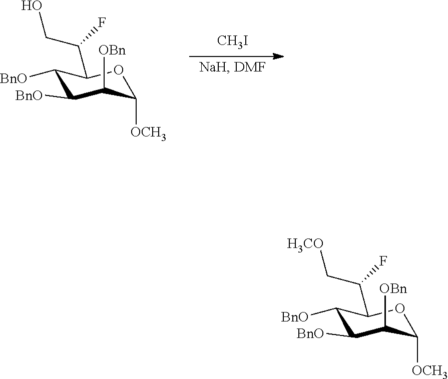

NaH (147.96 mg, 3.70 mmol, 60% purity) was added to the solution of (S)-2-fluoro-2-((2S,3S,4S,5S,6S)-3,4,5-tris(benzyloxy)-6-methoxytetrahydro-2H-pyran-2-yl)ethan-1-ol (1.67 g, 3.36 mmol) in DMF (10 mL) at 0° C. and stirred at 0° C. for 30 min, then CH₃I (572.83 mg, 4.04 mmol, 251.24 uL) was added at 0° C. and stirred at 20-25° C. for 2 h. The reaction was quenched with H₂O (40 mL) and extracted with EtOAc (30 mL×3), the organic phase was combined and washed with brine (50 mL×3), concentrated to give the crude product. The crude product was purified by column chromatography (silica gel, PE:EA=1:0 to 1:1) to give the desired product (1.6 g, yield: 89.5%) as colorless oil.

¹H NMR (400 MHz, CDCl₃) δ 7.42-7.28 (m, 15H), 5.11-5.06 (m, 0.5H), 5.01-4.94 (m, 1.5H), 4.78-4.74 (m, 3H), 4.69 (d, J=10.8 Hz, 1H), 4.61 (s, 2H), 4.20-4.13 (m, 1H), 3.90-3.80 (m, 2H), 3.80-3.77 (m, 1H), 3.70-3.64 (m, 1H), 3.63-3.57 (m, 1H), 3.41 (s, 3H), 3.28 (s, 3H).

MS (ESI) m/z (M+2Na⁺)=556.7.

### Step 2. Preparation of Compound 3S,4S,5S,6S)-3,4,5-tris(benzyloxy)-6-((S)-1-fluoro-2-methoxyethyl)tetrahydro-2H-pyran-2-yl Acetate

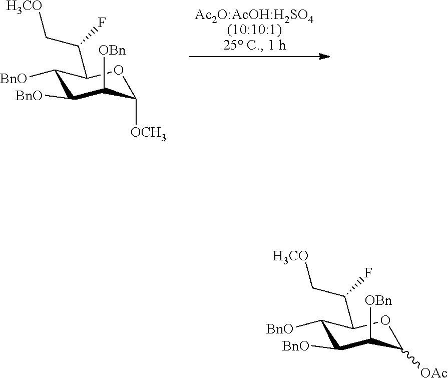

Con. H₂SO₄ (0.7 mL, 13.13 mmol) was added to the mixture of compound obtained in step 1 above (1.5 g, 2.94 mmol) in HOAc (7 mL) and Ac₂O (7 mL), stirred at 20-25° C. for 0.5 h. MeOH (3 mL) was added to quench the reaction under 0° C., then the reaction system was adjusted to pH 6-7 with sat. NaHCO₃ and extracted with EtOAc (40 mL×3). The organic phase was combined and concentrated to the desired compound (1.5 g, yield: 94.8%) as colorless oil.

MS (ESI) m/z (M+2Na)⁺=584.3.

### Step 3. Preparation of Compound (3S,4S,5S,6S)-6-((S)-1-fluoro-2-methoxyethyl)-3,4,5-trihydroxytetrahydro-2H-pyran-2-yl acetate

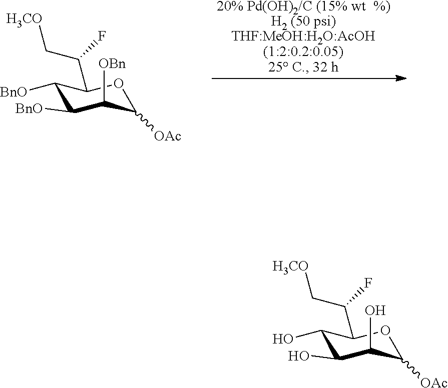

To the mixture of compound obtained from step 2 above (1.5 g, 2.78 mmol) and AcOH (0.1 mL, 1.75 mmol) in THF/MeOH/H₂O (v/v/v:1/2/1, 20 mL), Pd(OH)₂ (0.4 g, 284.83 umol, 20% purity, dry) was added and stirred at 25° C. for 16 h under H₂ atmosphere (50 psi). Filtered and the filtrate was concentrated to give the desired compound (800 mg, crude) as colorless oil. The crude product was used directly in next step without further purification.

¹H NMR (400 MHz, CD₃OD) δ5.99-5.93 (m, 1H), 5.02-4.96 (m, 0.5H), 4.88-4.86 (m, 0.5H), 3.95-3.84 (m, 1H), 3.83-3.79 (m, 1H), 3.74-3.60 (m, 2H), 3.59-3.46 (m, 2H), 3.41-3.33 (m, 3H), 2.14-2.07 (m, 3H).

### Step 4. Preparation of Compound (3S,4S,5S,6S)-6-((S)-1-fluoro-2-methoxyethyl)tetrahydro-2H-pyran-2,3,4,5-tetrayl tetraacetate

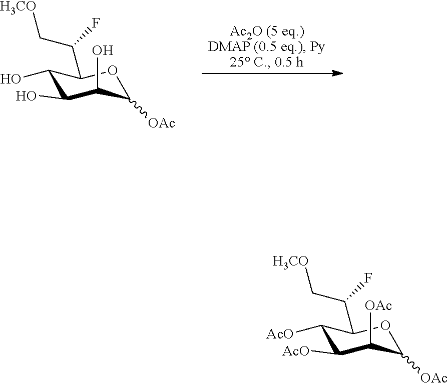

The mixture of compound obtained from step 3 above (800 mg, 2.98 mmol), Ac₂O (3.04 g, 29.82 mmol, 2.79 mL) and DMAP (36.44 mg, 298.24 umol) in DCM (5 mL) and pyridine (1 mL) was stirred at 20-25° C. for 16 h. MeOH (5 mL) was added to quenched the reaction and then the solvent was removed under reduced pressure to give the crude product. The crude product was purified by column chromatography (silica gel, PE:EA=1:0 to 1:1) to give the desired compound (710 mg, yield: 60.37%) as colorless oil.

¹H NMR (400 MHz, CDCl₃) δ 6.07 (d, J=1.3 Hz, 1H), 5.58-5.48 (m, 1H), 5.37-5.31 (m, 1H), 5.27-5.24 (m, 1H), 4.67 (t, J=6.4 Hz, 0.5H), 4.56 (t, J=6.4 Hz, 0.5H), 4.03-3.88 (m, 1H), 3.73-3.65 (m, 1H), 3.61-3.50 (m, 1H), 3.37 (s, 3H), 2.18 (s, 3H), 2.16 (s, 3H), 2.07 (s, 3H), 2.02 (s, 3H).

### Step 5. Preparation of Compound (2S,3S,4S,5S)-2-((S)-1-fluoro-2-methoxyethyl)-6-hydroxytetrahydro-2H-pyran-3,4,5-triyl triacetate

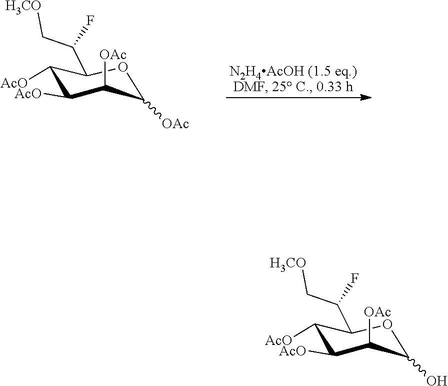

Acetic acid hydrazine (248.72 mg, 2.70 mmol, 1.5 eq) was added to the mixture of compound obtained from step 4 above (710 mg, 1.80 mmol, 1 eq) in DMF (5 mL) and stirred at 25° C. for 1 h. The reaction was diluted with H₂O (20 mL), extracted with EtOAc (30 mL×3), the organic phase was combined and washed with brine (40 mL×3), concentrated to give the crude product. The crude product was purified by column chromatography (silica gel, PE:EA=1:0 to 1:1) to give the desired compound (610 mg, 1.73 mmol, 96.17% yield) as colorless oil.

¹H NMR (400 MHz, CDCl₃) δ5.55-5.46 (m, 1H), 5.45-5.38 (m, 1H), 5.30-5.21 (m, 2H), 4.72 (m, 0.5H), 4.62-4.59 (m, 0.5H), 4.11-4.06 (m, 1H), 3.78-3.56 (m, 2H), 3.42 (s, 3H), 3.39-3.34 (m, 1H), 2.17 (s, 3H), 2.07 (s, 3H), 2.01 (s, 3H).

### Step 6 Preparation of Compound (2S,3S,4S,5S,6S)-2-((diphenoxyphosphoryl)oxy)-6-((S)-1-fluoro-2-methoxyethyl)tetrahydro-2H-pyran-3,4,5-triyl Triacetate

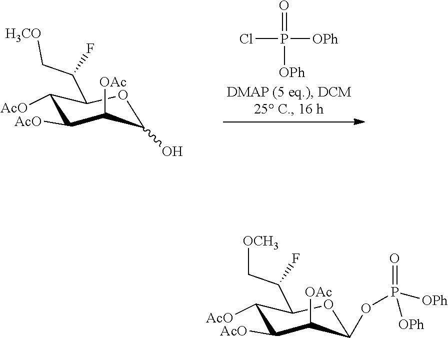

The mixture of compound diphenyl phosphorochloridate (1.40 g, 5.19 mmol) in DCM (10 mL) was added dropwise to the solution of compound obtained from Step 5 above (610 mg, 1.73 mmol) and DMAP (1.06 g, 8.66 mmol) in DCM (10 mL) at 10-20° C. during 30 min. After addition, the reaction mixture was stirred at 10-20° C. for 16 h. The reaction was diluted with DCM (50 mL), washed with HCl (1M, 40 mL), sat.NaHCO₃ (40 mL) and brine (40 mL), the organic phase was concentrated to give the crude product. The crude product was purified by column chromatography (silica gel, PE:EA=1:0 to 1:1) to give the desired compound (780 mg, yield: 62.51%) as colorless oil.

¹H NMR (400 MHz, CDCl₃) δ 7.39-7.09 (m, 10H), 5.55-5.43 (m, 3H), 5.07-4.99 (m, 1H), 4.66-4.60 (m, 0.5 H), 4.55-4.47 (m, 0.5H), 3.73-3.43 (m, 3H), 3.30 (s, 3H), 2.08 (s, 3H), 2.04 (s, 3H), 1.98 (s, 3H).

MS (ESI) m/z (M+Na⁺)=607.1.

### Synthesis of Compound 4

### Step 1. Preparation of Compound (2S,3S,4S,5S,6S)-2-((S)-2-acetoxy-1-fluoroethyl)-6-(((((S)-1-isopropoxy-1-oxopropan-2-yl)amino)(phenoxy)phosphoryl)oxy)tetrahydro-2H-pyran-3,4,5-triyl triacetate

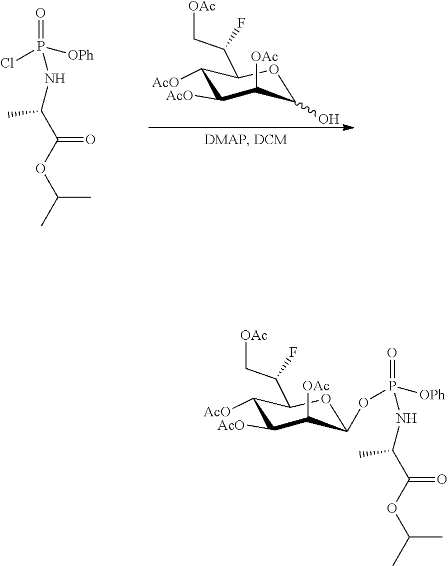

To a stirred solution of compound (2S,3S,4S,5S)-2-((S)-2-acetoxy-1-fluoroethyl)-6-hydroxytetrahydro-2H-pyran-3,4,5-triyl triacetate (200 mg, 526 μmol) and DMAP (321 mg, 2.63 mmol) in DCM (8 mL) was added compound isopropyl (chloro(phenoxy)phosphoryl)-L-alaninate (500.00 mg, 1.64 mmol, Ref. J. Med. Chem. 2017, 60, 3518-3524) in DCM (2 mL) slowly. The resulting mixture was stirred at 15° C. for 48 h. After completion of the reaction, the mixture was concentrated under reduced pressure. The crude product was purified by column chromatography (silica gel, PE:EA=1:0 to 3:7) to give the desired compound (80 mg, yield: 19.12% as a colorless syrup.

MS (ESI) m/z (M+Na)⁺=672.1

¹H NMR (400 MHz, CHLOROFORM-d) δ 7.37-7.26 (m, 2H), 7.22-7.09 (m, 3H), 5.73-5.29 (m, 3H), 5.20-4.91 (m, 2H), 4.79-4.52 (m, 1H), 4.44-4.11 (m, 2H), 4.07-3.90 (m, 1H), 3.90-3.72 (m, 1H), 3.72-3.54 (m, 1H), 2.20-2.10 (m, 3H), 2.10-2.01 (m, 6H), 2.01-1.92 (m, 3H), 1.43-1.29 (m, 3H), 1.26-1.18 (m, 6H).

### Compounds 6 and 7

### Chiral Separation of (2S,3S,4S,5S,6S)-2-((S)-2-acetoxy-1-fluoroethyl)-6-(((((S)-1-isopropoxy-1-oxopropan-2-yl)amino)(phenoxy)phosphoryl)oxy)tetrahydro-2H-pyran-3,4,5-triyl triacetate

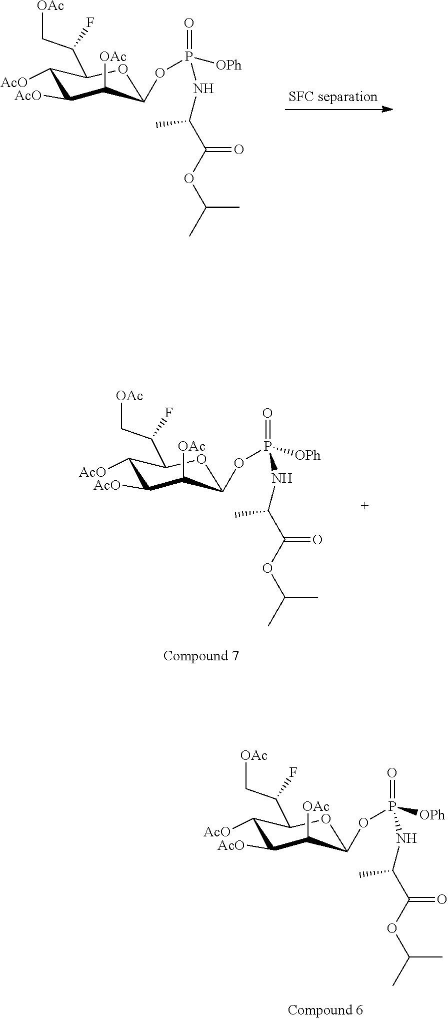

The compound obtained from Step 2 above (90 mg, 139 μmol) was purified by supercritical fluid chromatography (column: REGIS (s,s) WHELK-01 (250 mm*30 mm, 5 m); mobile phase: [0.1% NH₃H₂O EtOH]; B %: 30%-30%) to give two isomers (38 mg & 36 mg) as a white solid, Compounds 6 and 7.

### Compound 6

MS (ESI) m/z (M+Na)⁺=672.1

¹H NMR (400 MHz, CHLOROFORM-d) δ=7.30-7.20 (m, 2H), 7.17-6.96 (m, 3H), 5.47-5.26 (m, 3H), 5.10-4.89 (m, 2H), 4.75-4.50 (m, 1H), 4.34-4.24 (m, 2H), 4.02-3.87 (m, 1H), 3.86-3.72 (m, 1H), 3.68-3.52 (m, 1H), 2.09 (s, 3H), 2.03 (s, 3H), 1.99 (s, 3H), 1.90 (s, 3H), 1.35 (d, J=7.1 Hz, 3H), 1.23-1.15 (m, 6H).

### Compound 7

MS (ESI) m/z (M+Na)⁺=672.1

¹H NMR (400 MHz, CHLOROFORM-d) δ=7.37-7.27 (m, 2H), 7.22-7.13 (m, 3H), 5.63-5.51 (m, 2H), 5.46 (t, J=9.8 Hz, 1H), 5.13 (d, J=9.9 Hz, 1H), 5.05-4.93 (m, 1H), 4.74-4.53 (m, 1H), 4.36-4.16 (m, 2H), 4.01-3.90 (m, 1H), 3.78 (t, J=9.9 Hz, 1H), 3.72-3.61 (m, 1H), 2.17 (s, 3H), 2.06 (s, 3H), 2.04 (s, 3H), 1.99 (s, 3H), 1.35 (d, J=6.8 Hz, 3H), 1.25-1.17 (m, 6H).

### Synthesis of Compound 5

### Step 1. Preparation of Compound (2S,3S,4S,5S,6S)-2-((S)-2-acetoxy-1-fluoroethyl)-6-((bis((pivaloyloxy)methoxy)phosphoryl)oxy)tetrahydro-2H-pyran-3,4,5-triyl triacetate

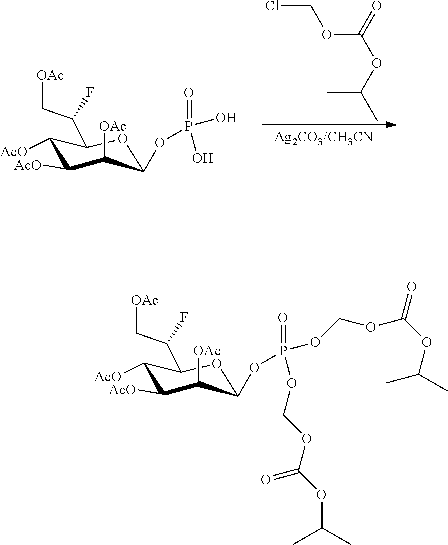

To a solution of (2S,3S,4S,5S,6S)-2-((S)-2-acetoxy-1-fluoroethyl)-6-(phosphonooxy)tetrahydro-2H-pyran-3,4,5-triyl triacetate (320 mg, 720.24 μmol) and chloromethyl isopropyl carbonate (769 mg, 5.04 mmol) in CH₃CN (10 mL) was added Ag₂CO₃ (596 mg, 2.16 mmol). The mixture was stirred at 70° C. for 12 h under N₂. The solid was filtered off. The filtrate was collected and concentrated. The residue was purified by column chromatography (silica gel, PE:EA=1: 0-0:1) to give the desired compound (32 mg, yield: 5.45%) as colorless oil.

MS (ESI) m/z [M+Na]⁺=715.1.

¹H NMR (400 MHz, CDCl₃) δ 5.66-5.40 (m, 7H), 5.13-5.10 (m, 1H), 4.98-4.84 (m, 2H), 4.78-4.71 (m, 0.5H), 4.66-4.59 (m, 0.5H), 4.44-4.33 (m, 2H), 3.78-3.66 (m, 1H), 2.17 (s, 3H), 2.07 (s, 3H), 2.04 (s, 3H), 1.97 (s, 3H), 1.34-1.27 (m, 12H).

### Synthesis of Compound 8

### Step 1. Preparation of Compound (2S,3S,4S,5S,6R)-2-((S)-2-acetoxy-1-fluoroethyl)-6-((4-(3-chlorophenyl)-2-oxido-1,3,2-dioxaphosphinan-2-yl)oxy)tetrahydro-2H-pyran-3,4,5-triyl Triacetate

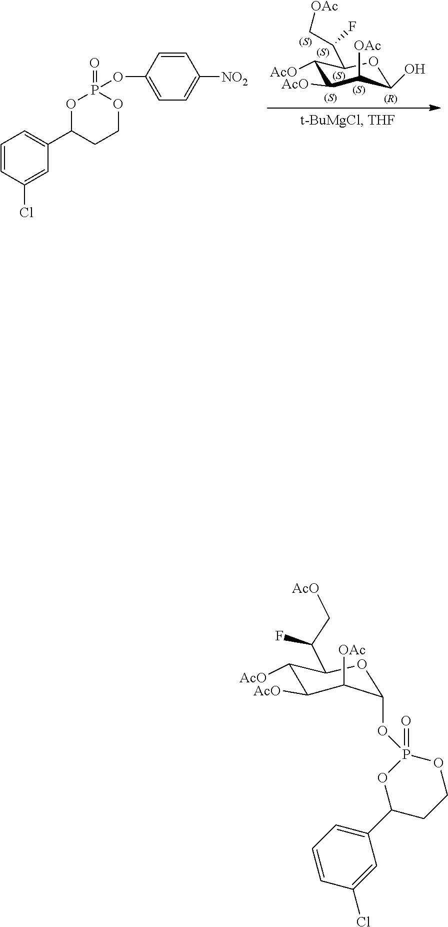

To the solution of compound (2S,3S,4S,5S,6R)-2-((S)-2-acetoxy-1-fluoroethyl)-6-hydroxytetrahydro-2H-pyran-3,4,5-triyl triacetate (100 mg, 262.9 μmol) in THF (4 mL) under nitrogen was treated with t-BuMgCl (1.7 M, 464.0 uL) at 0° C. The mixture was stirred at 0° C. for 0.5 hr. Then compound 4-(3-chlorophenyl)-2-(4-nitrophenoxy)-1,3,2-dioxaphosphinane 2-oxide (Ref: J. AM. CHEM. SOC. 9 VOL. 126, NO. 16, 2004) (126.4 mg, 341.8 μmol) was added and the mixture was stirred at 15° C. under nitrogen for 16 hr. The reaction was quenched with sat. NH₄Cl (10 mL). The mixture was extracted with ethyl acetate (15 mL×3). The organic layer was dried over Na₂SO₄ and concentrated under vacuum. The residue was purified by column chromatography (silica gel, EA:PE=1:3 to 7:3) twice. The desired compound (20.96 mg, yield: 11.9%) was obtained as a white solid.

¹H NMR (400 MHz, CDCl₃) δ 7.40-7.27 (m, 4H), 5.78-5.67 (m, 2H), 5.54-5.51 (m, 1H), 5.37-5.34 (m, 2H), 4.75-4.36 (m, 5H), 4.13-4.09 (m, 2H), 2.42-2.35 (m, 1H), 2.18 (s, 3H), 2.07-2.04 (m, 6H), 2.02-2.01 (m, 3H).

MS (ESI) m/z (M+Na)⁺=633.0.

### Synthesis of Compound 10 and 11

### Step 1. Preparation of Alfa Isomer and Beta Isomer of Compound (2S,3S,4S,5S)-2-((S)-1-fluoro-2-methoxyethyl)-6-(((((S)-1-isopropoxy-1-oxopropan-2-yl)amino)(phenoxy)phosphoryl)oxy)tetrahydro-2H-pyran-3,4,5-triyl Triacetate

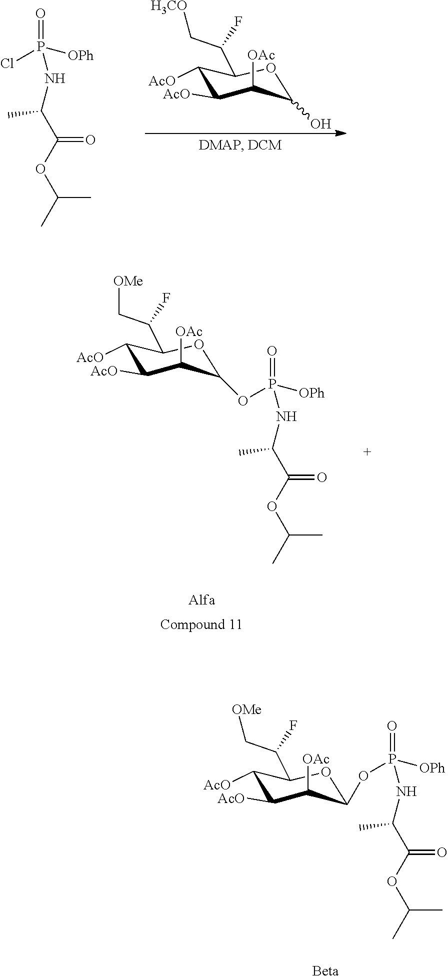

The solution of compound isopropyl (chloro(phenoxy)phosphoryl)-L-alaninate (1.2 g, Ref. J. Med. Chem. 2017, 60, 3518-3524) in DCM (3 mL) was added dropwise to the solution of compound (2S,3S,4S,5S)-2-((S)-1-fluoro-2-methoxyethyl)-6-hydroxytetrahydro-2H-pyran-3,4,5-triyl triacetate (300 mg, 851.53 μmol) and DMAP (520.15 mg, 4.26 mmol) in DCM (5 mL) at 10-20° C., then the reaction was stirred at 10-20° C. for 4 h. The reaction was diluted with DCM (20 mL), washed with HCl (1M, 10 mL), sat.NaHCO₃ (10 mL) and brine (10 mL). The obtained organic phase was concentrated to give the crude product. The crude product was purified by column chromatography (silica gel, PE:EA=1:0 to 1:2) to afford crude compound 11 (as Alfa isomer) (250 mg, yield: 24.3%, 51.5% purity) which was purified by Pre-HPLC (YMC Triart C18 150*25 mm*5 m, water (10 mM NH₄HCO₃)-ACN, 53% to 83%) to give compound A (150 mg, yield: 43.7%) as a white solid.

¹H NMR (400 MHz, CDCl₃) 7.41-7.32 (m, 2H), 7.27-7.17 (m, 3H), 5.76 (d, J=6.6 Hz, 1H), 5.60-5.48 (m, 1H), 5.41-5.27 (m, 2H), 5.10-4.99 (m, 1H), 4.74-4.49 (m, 1H), 4.25-3.89 (m, 2H), 3.82-3.43 (m, 3H), 3.39 (s, 1H), 3.29 (s, 2H), 2.18 (s, 3H), 2.10-2.05 (m, 3H), 2.01 (s, 3H), 1.46-1.37 (m, 3H), 1.29-1.21 (m, 6H). MS (ESI) m/z (M+2Na⁺)=667.2. And compound Beta (140 mg, yield: 13.8%) as colorless oil. MS (ESI) m/z (M+Na⁺)=644.1

### Chiral Separation of Beta Isomer of Compound (2S,3S,4S,5S)-2-((S)-1-fluoro-2-methoxyethyl)-6-(((((S)-1-isopropoxy-1-oxopropan-2-yl)amino)(phenoxy)phosphoryl)oxy)tetrahydro-2H-pyran-3,4,5-triyl Triacetate

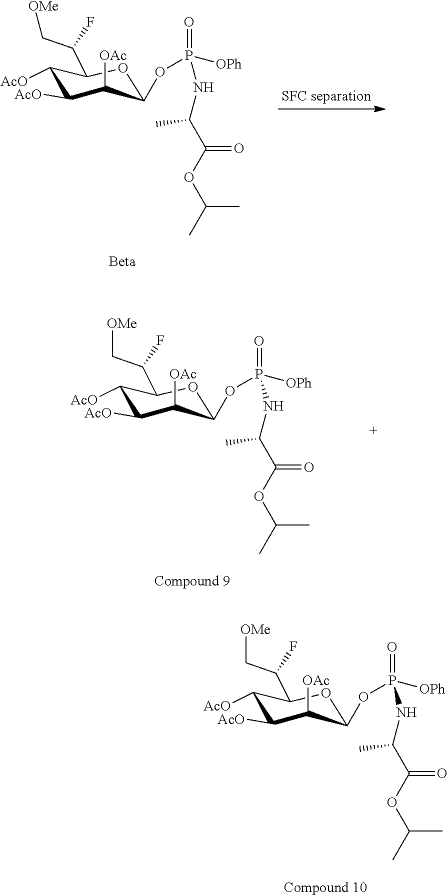

Compound Beta (140 mg) was purified by SFC separation ((s,s) WHELK-O1 (250 mm*50 mm, 10 um)), 0.1% NH₃H₂O EtOH, 30% to 30%) to give compound 9 (57.0 mg, yield: 40.71%, Rt=3.209) and compound 10 (40.3 mg, yield: 28.79%, Rt=3.857) both as white solid.

### Compound 9

¹H NMR (400 MHz, CDCl₃) δ 7.38-7.29 (m, 2H), 7.25-7.13 (m, 3H), 5.64-5.54 (m, 2H), 5.49 (t, J=10.0 Hz, 1H), 5.15 (dd, J=3.0, 10.0 Hz, 1H), 5.07-4.96 (m, 1H), 4.69-4.62 (m, 0.5H), 4.57-4.51 (m, 0.5H), 4.03-3.91 (m, 1H), 3.84-3.74 (m, 1H), 3.73-3.48 (m, 3H), 3.32 (s, 3H), 2.20 (s, 3H), 2.06 (s, 3H), 2.02 (s, 3H), 1.35 (d, J=7.1 Hz, 3H), 1.23 (dd, J=6.4, 10.4 Hz, 6H). MS (ESI) m/z (M+2Na⁺)=667.2.

### Compound 10

¹H NMR (400 MHz, CDCl₃) δ 7.38-7.29 (m, 2H), 7.22-7.12 (m, 3H), 5.50-5.39 (m, 3H), 5.11-5.00 (m, 2H), 4.71-4.64 (m, 0.5H), 4.59-4.52 (m, 0.5H), 4.10-3.98 (m, 1H), 3.92-3.84 (m, 1H), 3.80-3.59 (m, 3H), 3.42 (s, 3H), 2.17 (s, 3H), 2.06 (s, 3H), 1.98 (s, 3H), 1.43 (d, J=7.1 Hz, 3H), 1.27 (dd, J=6.4, 9.5 Hz, 6H). MS (ESI) m/z (M+2Na⁺)=667.2.

### Synthesis of Compound 12

### Step 1. Preparation of Compound (2S,3S,4S,5S,6S)-2-((S)-2-acetoxy-1-fluoroethyl)-6-((hydroxy(phenoxy)phosphoryl)oxy)tetrahydro-2H-pyran-3,4,5-triyl triacetate

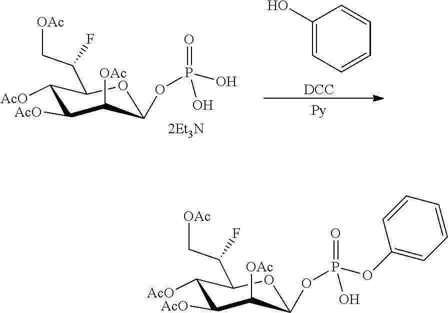

To a solution of compound (2S,3S,4S,5S,6S)-2-((S)-2-acetoxy-1-fluoroethyl)-6-(phosphonooxy)tetrahydro-2H-pyran-3,4,5-triyl triacetate (230 mg, 347.08 μmol, 2Et₃N) in DMF (5 mL) and pyridine (1 mL) was added DCC (214.84 mg, 1.04 mmol) followed by the addition of phenol (39.20 mg, 416.50 μmol). The reaction was stirred at 100° C. for 16 h under N₂ atmosphere. The solvent was removed under reduced pressure and the residue was purified by column chromatography (silica gel, DCM:MeOH=1:0 to 10:1) to give crude product (100 mg). The crude product (100 mg) was purified by Pre-HPLC (Welch Xtimate C18 150*25 mm*5 μm, water (10 mM NH₄HCO₃)-ACN, 15% to 45%) to give the desired compound (4.65 mg, yield: 2.35%) as a white solid.

¹H NMR (400 MHz, CD₃OD) δ 7.31-7.19 (m, 4H), 7.09-7.01 (m, 1H), 5.51-5.34 (m, 4H), 5.20 (dd, J=3.3, 9.8 Hz, 1H), 4.77-4.72 (m, 0.5H), 4.65-4.60 (m, 0.5H), 4.35-4.24 (m, 1H), 3.91-3.80 (m, 1H), 2.12 (s, 3H), 2.06-2.03 (m, 6H), 1.94 (s, 3H). MS (ESI) m/z (M+2Na⁺)=582.1.

### Synthesis of Compound 13 and 14

### Step 1. Preparation of Compound (2S,3S,4S,5S,6S)-2-((S)-2-acetoxy-1-fluoroethyl)-6-((hydroxy(pyridin-3-yloxy)phosphoryl)oxy)tetrahydro-2H-pyran-3,4,5-triyl triacetate

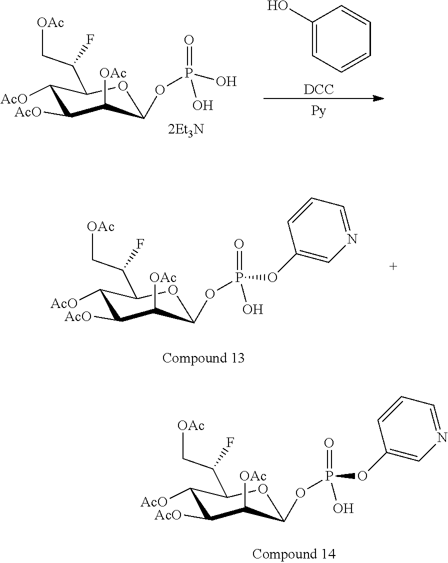

To the mixture of compound (2S,3S,4S,5S,6S)-2-((S)-2-acetoxy-1-fluoroethyl)-6-(phosphonooxy)tetrahydro-2H-pyran-3,4,5-triyl triacetate (140 mg, 211.3 mol, 2Et₃N) in pyridine (0.5 mL) and DMF (3 mL) were added DCC (435.9 mg, 2.1 mmol, 427.4 μL) and pyridin-3-ol (100.5 mg, 1.1 mmol). The mixture was stirred at 100° C. under nitrogen for 16 hr. The mixture was concentrated under vacuum. The residue was purified by column chromatography (silica gel, DCM:methanol=1:0 to 10:1) and further purified by Prep-HPLC (column: Welch Xtimate C18 150*25 mm*5 m; mobile phase: [water (10 mM NH₄HCO₃)-ACN]; B %: 10%-40%, 10 min) to give two isomers:

### Compound 13

(5.85 mg, 10.9 μmol, Yield: 5.2%, 100% purity) was obtained as a white solid. ¹H NMR (400 MHz, CD3OD) δ 8.47 (s, 1H), 8.21 (d, J=4.4 Hz, 1H), 7.71 (d, J=7.2 Hz, 1H), 7.38-7.35 (m, 1H), 5.37-5.34 (m, 3H), 5.21-5.19 (m, 1H), 4.60-4.59 (m, 2H), 4.29-4.25 (m, 1H), 3.90-3.88 (m, 1H), 2.14 (s, 3H), 2.10-2.04 (m, 6H), 1.92 (s, 3H). MS (ESI) m/z (M+H)⁺=538.1.

### Compound 14

(10.78 mg, 18.9 mol, Yield: 9.0%, 94.29% purity) was obtained as a white solid. ¹H NMR (400 MHz, CD3OD) δ 8.52 (s, 1H), 8.28 (s, 1H), 7.86 (d, J=8.4 Hz, 1H), 7.49 (s, 1H), 5.48-5.45 (m, 2H), 5.36 (t, J=10.0 Hz, 1H), 5.22-5.19 (m, 1H), 4.75-4.67 (m, 1H), 4.29-4.25 (m, 2H), 3.90-3.81 (m, 1H), 2.10 (s, 3H), 2.03 (s, 6H), 1.92 (s, 3H). MS (ESI) m/z (M+H)⁺=538.2.

### Synthesis of Compound HMP1BP (D-glycero-D-manno-heptose-1βP, Compound 1)

### Step 1. Synthesis of Compound II

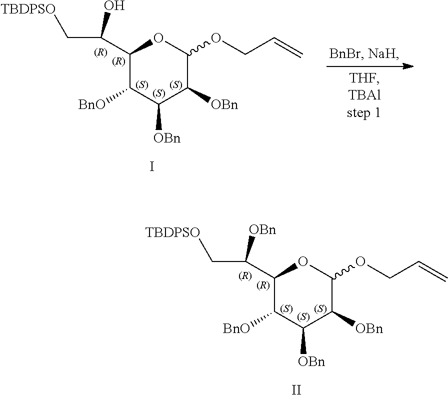

To a stirred mixture of compound I (17.93 g, 23.65 mmol), TBAI (0.9 g, 2.365 mmol) and BnBr (7.1 mL, 59.14 mmol) in DMF (270 mL) was added NaH (60% oil dispersion, 2.4 g, 59.14 mmol) at 0° C. After stirring overnight, the reaction was quenched with H₂O. The whole mixture was extracted with PE/EtOAc (1:9). The extract was washed with H₂O and brine, and dried over MgSO₄. The filtrate was concentrated under reduced pressure to give an oily residue, which was purified by flash chromatography over silica gel with PE-EtOAc (5:1) to give compound II (6.3407 g, 32% yield) as a colorless oil ¹H NMR (CDCl₃, 400 MHz) δ (ppm): ¹H NMR (CDCl₃, 400 MHz) 1.04 (s, 9H); 3.75˜3.77 (m, 1H); 3.84˜3.96 (m, 5H); 2.44˜2.47 (d, 1H); 4.05˜4.14 (m, 3H); 4.56˜4.86 (m, 8H); 5.10˜5.21 (m, 2H); 5.79˜5.84 (m, 1H); 7.02˜7.05 (m, 2H), 7.16˜7.38 (m, 24H); 7.60˜7.67 (m, 4H)

### Step 2. Synthesis of Compound III

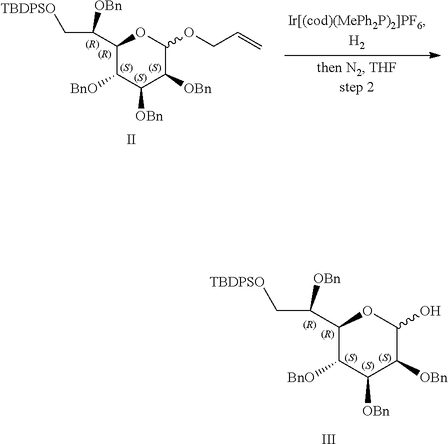

A solution of Ir[(cod)(MePh₂P)₂]PF₆ (210 mg, 253 mmol) in THF (35 mL) was stirred at room temperature under 1 atm H₂ atmosphere until a light yellow solution was generated, then N₂ was bubbled through the solution to remove any residual hydrogen gas. The resulting solution of Ir catalyst was added to a stirred solution of compound II (1.0741 g, 1.27 mmol) in THF (35 mL) at room temperature. After stirring for 6 h at this temperature, H₂O (22 mL) and 12 (650 mg, 2.56 mmol) was added to the stirred mixture at room temperature. After stirring for 1 h at this temperature, the reaction was quenched with saturated Na₂S₂O₃. The whole mixture was extracted with EtOAc. The extract was washed with saturated NaHCO₃ and dried over with MgSO₄. The filtrate was concentrated under reduced pressure to give an oily residue, which was purified by flash chromatography over silica gel with PE-EtOAc (1:1) to give compound III (0.68 g, 66.3% yield) as a colorless oil.

¹H NMR (CDCl₃, 400 MHz) δ (ppm): 1.03 (s, 9H), 3.72 (s, 1H); 3.93˜4.05 (m, 6H); 4.23˜4.45 (m, 1H); 4.55˜4.80 (m, 7H); 5.13 (br, 1H); 7.02˜7.07 (m, 2H); 7.21˜7.37 (m, 24H); 7.62˜7.68 (m, 4H).

### Step 3. Synthesis of Compound IV and V

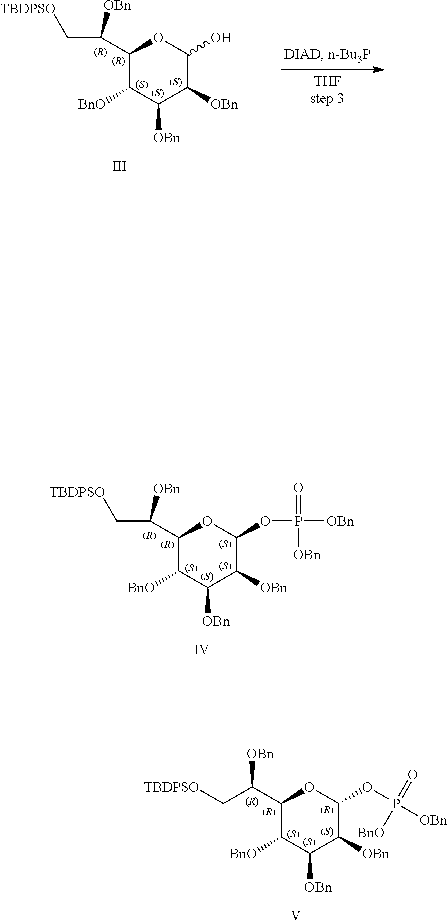

To a stirred mixture of compound III (680 mg, 0.842 mmol), dibenzyl phosphate (702 mg, 2.53 mmol), n-Bu₃P (0.51 g, 2.53 mmol) and MS 5 Å (500 mg) in CH₂Cl₂ (20 mL) was added Et₃N (0.71 mL, 5.06 mmol) at room temperature. After stirring for 30 min at this temperature, DIAD (0.51 g, 2.53 mmol) was added at room temperature. After stirring overnight, the mixture was concentrated under reduced pressure to give an oily residue. The crude product was purified by flash chromatography over silica gel with PE-EtOAc (7:3) to give a mixture of compound IV and V (0.966 g, 100%) which was used in next step directly.

### Step 4. Synthesis of Compound VI and VII

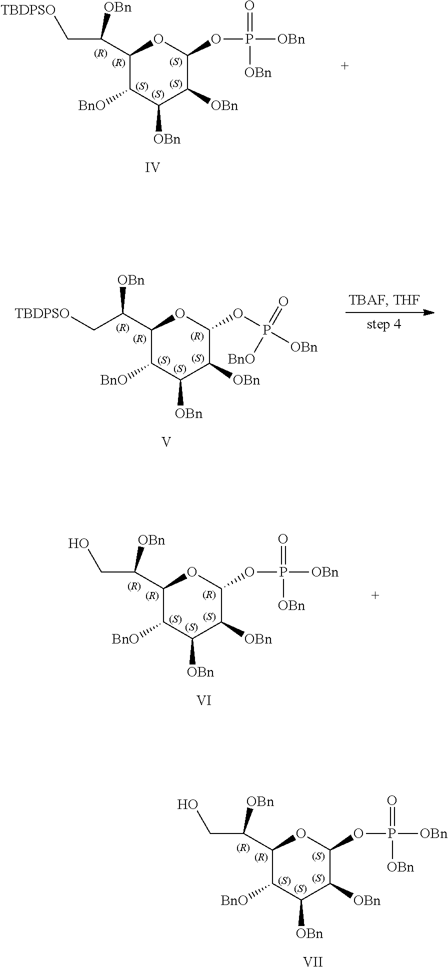

To a stirred solution of a mixture of compounds IV and V (0.966 g, 0.904 mmol) in THF (20 mL) was added TBAF (1 M in THF, 1.4 mL, 1.4 mmol) at room temperature. After stirring overnight, the reaction was quenched with saturated NH₄C1. The whole mixture was extracted with EtOAc. The extract was washed with saturated NaHCO₃ and dried over MgSO₄. The filtrate was concentrated under reduced pressure to give an oily residue, which was purified by flash chromatography with petroleum/EtOAc (3:1) to give compound VI (169.7 mg, 22.6%) and compound VII (225 mg, 30%) as a colorless oil. Compound VI: ¹H NMR (CDCl₃, 400 MHz) δ (ppm): 3.52-3.54 (m, 1H); 3.67˜3.73 (m, 2H), 3.84˜3.87 (dd, 1H); 3.97˜3.99 (m, 1H); 4.04˜4.07 (m, 1H); 4.47˜4.50 (m, 1H); 4.58˜4.60 (m, 1H); 4.71˜4.74 (m, 1H); 4.87˜4.89 (m, 1H); 4.93˜5.03 (m, 9H); 5.70˜5.72 (dd, 1H); 7.19˜7.33 (m, 30H). ³¹P NMR (CDCl₃, 400 MHz) δ −2.60. Compound VII: 1H NMR (CDCl₃, 400 MHz) δ3.56˜3.59 (dd, 1H); 3.65˜3.68 (m, 2H); 3.81˜3.84 (m, 2H); 4.02˜4.07 (m, 1H); 4.52˜4.56 (m, 1H); 4.59˜4.61 (m, 1H); 4.68˜4.78 (m, 3H); 4.86˜4.88 (m, 1H); 4.95˜5.11 (m, 7H); 5.24˜5.26 (d, 1H); 7.18˜7.39 (m, 30H). ³¹P NMR (CDCl₃, 400 MHz) δ −2.50

### Step 5. Synthesis of Compound VIII (D-glycero-D-manno-heptose-1β-P)

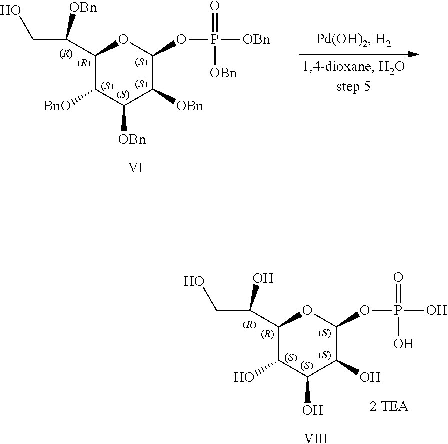

A mixture of compound VI (105 mg, 0.126 mmol) and 20% w/w Pd(OH)₂/C (21 mg, 0.03 mmol) in 1,4-dioxane/H₂O (5 mL, 4:1) was stirred at room temperature under H₂ (1 atm) for 2 days. The mixture was filtrated through an Advantech PTFE membrane filter with a pore size of 0.5 m with H₂O. The filtrate was cooled to 0° C. and was added TEA (53 uL, 0.378 mmol) and stirred at this temperature for 3 h. The resulting mixture was lyophilized to give compound VIII-2Et₃N as a white solid (74.3 mg, quant.).

The invention is further described and exemplified by the following non-limiting examples.

### EXAMPLES

### Example 1: Compound 2 has Unexpected Biological Activity in Liver Cells Compared to H1BADP (Compound 1)

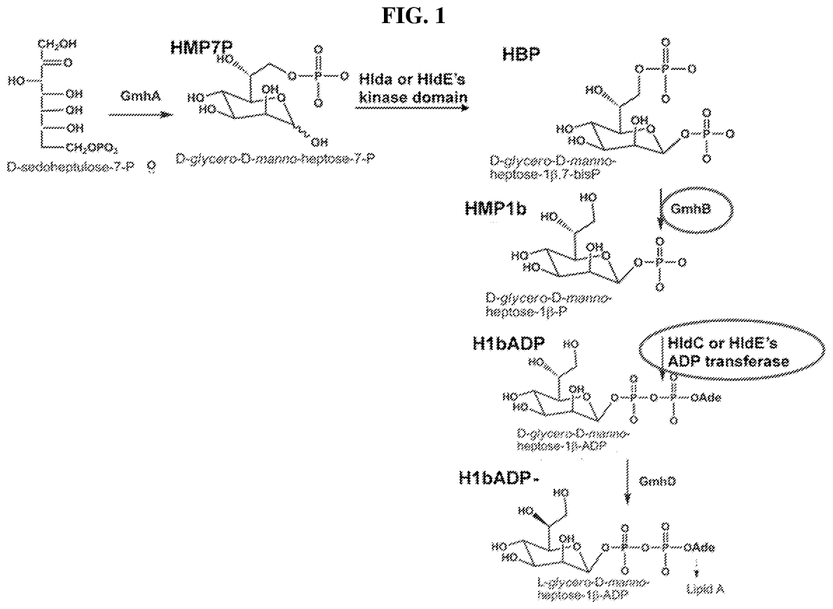

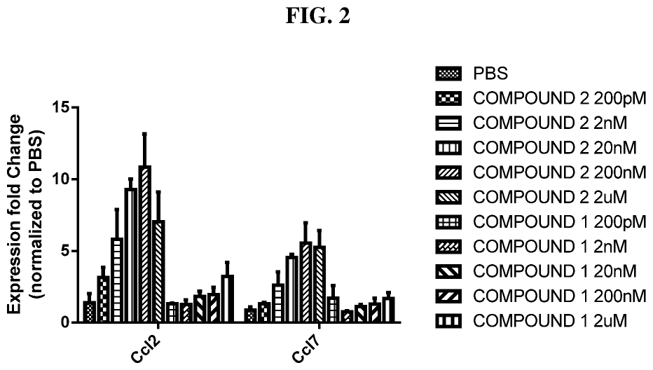

The reference compound, referred to herein also as “Compound 1” (HMP1BP) is an intermediate in the H1b-ADP biosynthetic pathway generated by the dephosphorylation of HBP (FIG. 1). Derivatives of Compound 1 were generated and their activities tested in primary mouse hepatocytes (C57/b6). One such derivative, Compound 2, exhibited unexpected biological activity in liver cells compared to Compound 1. Hepatocytes were first isolated from fresh C57/b6 mouse livers, transferred to serum-free medium, and cultured overnight before treatment with Compound 1 or Compound 2 for 4 hours. Liver cells were then harvested, the mRNA isolated and gene expression of C—C motif chemokine ligand 2 (CCL2) and C—C motif chemokine ligand 7 (CCL7) was analyzed by qPCR and presented as fold change compared to a no treatment (PBS) control. Gene expression was normalized to the expression of the housekeeping gene, glyceraldehyde-3-phosphate dehydrogenase (GAPDH). As shown in FIG. 2, Compound 2 significantly induced CCL2 expression in a dose-dependent manner in a range of from 200 picomolar to 200 nanomolar. In contrast, Compound 1 did not significantly induces CCL2 expression relative to the PBS control except at the highest concentration tested, which was 2 micromolar. Even at this high concentration, the gene expression induced by Compound 1 was only similar to that induced by the lowest concentration (200 picomolar) of Compound 2. In this assay.

CCL7 gene expression was also induced in a dose-dependent manner at between 2 nanomolar and 200 nanomolar Compound 2, while Compound 1 showed only a much smaller induction in CCL7 gene expression at the two highest doses, 200 nanomolar and 2 micromolar.

One possibility for the unexpected increase in gene expression observed with Compound 2 is that the modification of the hydroxyl group of HMP1BP with hydrophobic groups, such as benzene, allowed Compound 2 to enter into the liver cells much more efficiently than Compound 1.

### Example 2: Compound 2 Induces Chemokine and Cytokine Expression in Mouse Liver Via ALPK1

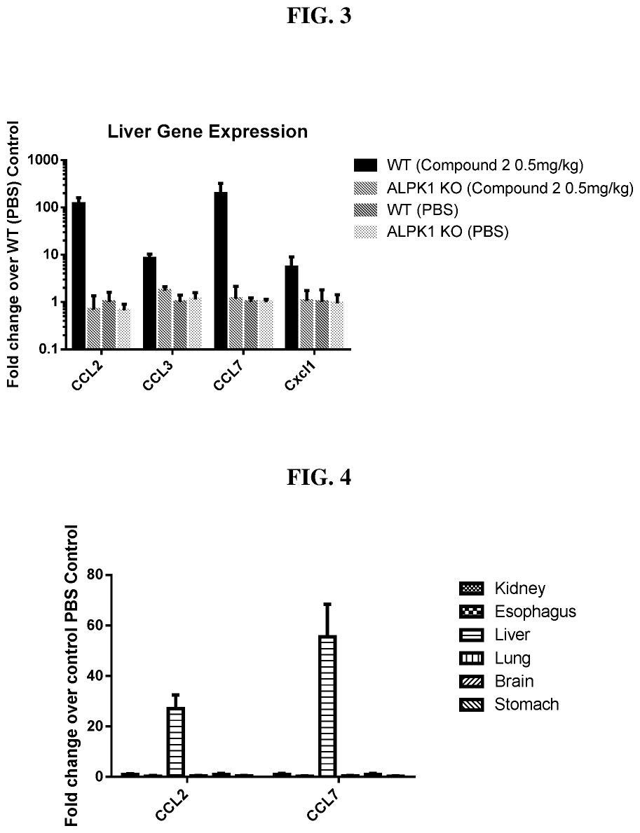

ALPK1 knockout (KO) mice and wildtype (WT) control were treated orally with either PBS or Compound 2 (0.5 mg/kg). Four hours after treatment, livers were dissected for gene expression analysis by qPCR. Expression was normalized to PBS treated WT mice. As shown in FIG. 3, Compound 2 treatment induce the expression of each of CCL2, CCL3, CCL7, and CXCL1 in WT mice but not ALPK1 KO mice, indicating that Compound 2 requires ALPK1 in order to stimulate gene expression of these chemokines.

### Example 3: Compound 2 Activates Chemokines Only in Liver Cells Following Oral Administration to Mice

FIG. 4 shows that when 8 week-old C57 female mice were administered Compound 2 diluted in 200 μM saline and 1.5% DMSO by oral gavage, only liver cells showed strong CCL2 and CCL7 gene expression as measured by qPCR. In this experiment, CCL7 was expressed more than 50-fold above the PBS control and CCL2 was expressed more than 20-fold above the control. No other organ tissue analyzed showed cytokine and chemokine gene expression.

### Example 4: Other Compound 1 Derivatives Activate Cytokines and Chemokines in Liver Cells Following Oral Administration to Mice

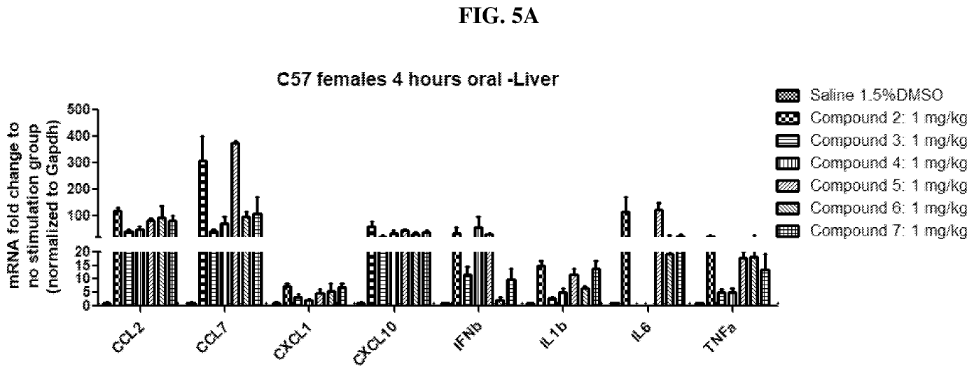

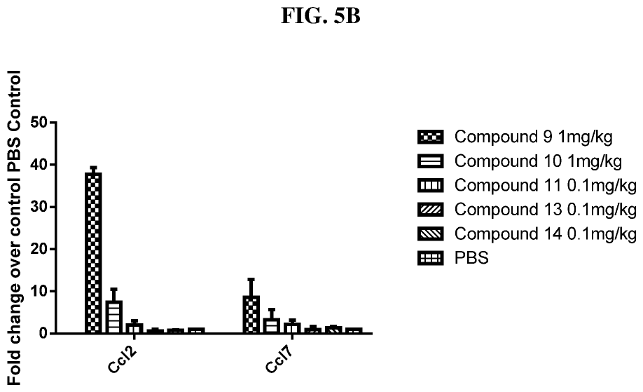

Several additional Compound 1 derivatives were tested for cytokine and chemokine gene induction in hepatocytes. FIG. 5 shows an expanded set of chemokine and cytokine genes and their expression profiles when exposed to Compounds 2-7, and for Compounds 9-11 and 13-14, CCL2 and CCL7 only. Mice were treated as described above. Briefly, 8 week-old C57 female mice were separately administered (1 mg/kg) either one of Compounds 2-7 or a saline/DMSO control by oral gavage and 4 hours later the livers were dissected and gene expression analyzed by qPCR. The gene expression results were normalized to GADPH expression. As shown in FIG. 5A, in all cases, the Compound 1 derivatives induced cytokine and chemokine gene expression above the control. The additional genes tested in this experiment are: CXCL1, encoding C—X—C motif chemokine ligand 1; CXCL10, encoding C—X—C motif chemokine ligand 10; IFNb, encoding interferon beta; IL-1b, encoding interleukin 1 beta; IL6, encoding interleukin 6; and TNFα, encoding tumor necrosis factor alpha. Only CXCL1 and IL1b did not produce a strong inductive effect with any of the Compound 1 derivatives. All the derivatives induced CCL2 and CCL7 more than 20-fold above the control. Compound 2 induced CCL2, CCL7, CXCL10, IFNb, IL6 and TNFα each more than 20-fold. CXCL10 was also highly induced by all of the Compound 1 derivatives. Similar to Compound 2, Compounds 4 and 5 had a strong inductive effect on IFNb and Compound 5 had a strong inductive effect on IL6.

When Compounds 9-10 (1 mg/kg) and Compounds 11, 13-14 (0.1 mg/kg) were administered by oral gavage as described above, Compound 9 exhibited the strongest effects on CCL2 gene expression (FIG. 5B). Compound 10 also exhibited a strong induction of CCL2 gene expression.

### Example 5: Compound 2 Leads Treatment Reduces HBV DNA, HBsAg and HBeAg in Serum in a Mouse Model

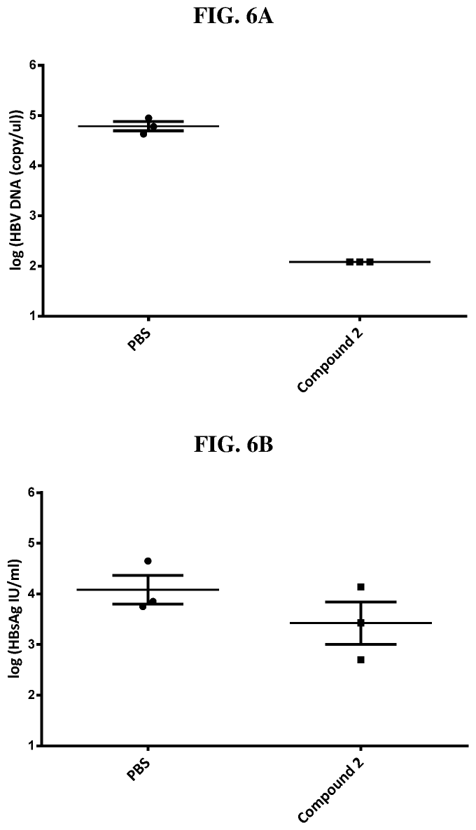

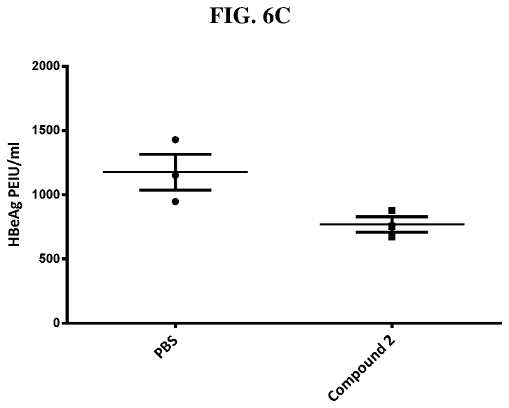

On day 1, male C57BL/6 mice were intravenously (i.v) injected with hepatitis B virus AAV8-1.3 HBV (1×10¹¹ v/g) (Beijing Five Plus Molecular Medicine Institute). On day 56 post-injection, each mouse was treated with Compound 2 two times per week (1 mg/kg per dose) or PBS. After seven days of treatment (at day 63 post injection), mouse serum was collected for analysis by qPCR (HBV DNA) and ELISA for Hepatitis B virus surface antigen (HBsAg) and Hepatitis B e-antigen (HBeAg). FIG. 6A shows that Compound 2 significantly reduced HBV DNA copy number. Hepatitis B surface antigen (FIG. 6B) and Hepatitis B e-antigen (FIG. 6C) were also both decreased compared to the PBS control. Collectively, these results show an unexpected specificity and activity of derivatives of Compound 1 in liver cells. Specifically, the Compound 1 derivatives show tissue-specific liver cell activity and strong induction of cytokine and chemokine genes while also reducing HBV DNA and antigen titers in the serum of infected mice.

Those skilled in the art will recognize or be able to ascertain using no more than routine experimentation, many equivalents to the specific embodiments of the invention as described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A compound represented by formula (I): 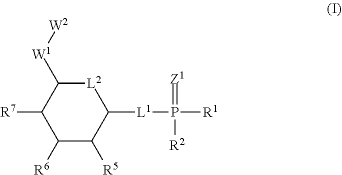
and/or a stereoisomer, tautomer, stable isotopes, prodrug or pharmaceutically acceptable salt thereof, wherein:
L¹ is selected from O, S, CH₂, CHF, CF2, OCH₂, SCH₂, OCHF, SCHF, OCF₂ or SCF₂;
L² is selected from the group consisting of O, S, CH₂, NR, CH₂, CH(OH), CHF and CF₂, wherein R is H or C1-C8 alkyl substituted with 0-3 substituents selected from halo, —OH, ═O, C1-C4 alkoxy, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members;
Z¹ is selected from O and S;
W¹ is —C(R¹⁰R¹¹)—, wherein R¹⁰ and R¹¹ are independently selected from H, D, —OH, halogen, and optionally substituted groups selected from C1-C4 alkyl, C1-C4 alkoxyl, C1-C4 haloalkyl, C1-C4-haloalkoxy, C1-C4 alkenyloxy, aralkyloxy, and 1-6 membered oligopeptidyl linked via C-termional C(O)O— and R¹²CO₂—, wherein R¹² is selected from C1-C20 alkyl, C1-C20 alkenyl, C1-C20 alkoxy, C1-C20 alkenyloxy, C1-C20 alkylamino, C3-C6 cycloalkyl, heterocyclyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members and 1-6 membered oligopeptidyl linked via N-terminal N; wherein the optional substituents for R¹⁰ and R¹¹ are 1-3 substituents independently selected from D, halogen, —OH, ═O, C1-C4 alkyl and C1-C4 alkoxy;
W² is R¹³-Q¹-W³—, wherein Q¹ is selected from —O— or —NH—; W³ is selected from a bond or C1-C3 alkylene groups optionally substituted with 1-3 substituents independently selected from halogen, —OH, ═O, C1-C3 alkoxy, C1-C3 haloalkyl, C1-C3 haloalkoxyl, C1-C3 alkenyloxy; wherein R¹³ is 1-6 membered oligopeptidyl linked via C-terminal carbonyl group or R¹⁴Q²C(O)—; wherein Q² is a bond, —O— or —NH—; R¹⁴ is 1-6 membered oligopeptidyl linked via N-terminal N or an optionally substituted group selected from C1-C20 alkyl, C1-C20 alkylenyl, C1-C20 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members, and R¹⁴ is R¹⁵-Q³-Q⁴-Q⁵-; wherein Q³, Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy;
R¹ and R² are independently selected from the group consisting of —OR^{a}, and —NR^{b}R^{c}; when both R¹ and R² are —OR^{a}, the R^{a} moieties can combine to form a five or six-membered heterocyclic ring, wherein
the five or six-membered heterocyclic ring is substituted with from 0 to 3 R³ moieties selected from the group consisting of H, D, halogen, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members, wherein the aryl or the 5 or 6 membered heteroaryl are substituted with 0 to 3 R^{3a} substituents selected from the group consisting of halogen and C1-C8 alkyl; or
when two R³ substituents are on adjacent ring vertices of the five or six-membered heterocyclic ring, they can combine to form a fused phenyl ring, which is substituted with from 0 to 3 R⁴ moieties selected from the group consisting of H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R^{a} is selected from the group consisting of H, D, C1-C12 alkyl, C1-C12 haloalkyl, —C(R^{a1})(R^{a2})C(O)OR^{a3}, —C(R^{a1})(R^{a2})OC(O)R^{a3}, 3 to 6 membered heterocycloalkyl having 1-3 heteroatoms selected from N, O, and S as ring members, aryl, 5 to 10 membered heteroaryl, —C1-C4 alkylene-aryl, and —C1-C4 alkylene-5 to 10 membered heteroaryl,
wherein the 5 or 10 membered heteroaryl has 1-3 heteroatoms selected from the group consisting of O, N, and S as ring members and the 5 or 10 membered heteroaryl is substituted with from 0 to 2 substituents selected from the group consisting of halogen, C1-C8 alkyl, and —NO₂,
each R^{b} and R^{c} are independently selected from the group consisting of H, C1-C12 alkyl, C1-C12 alkoxyl, C1-C12 alkanoyloxyl, C1-C12 alkenyloxyl, C3-C6 cycloalkyl, 4 to 6 membered heterocyclyoalkyl having 1-3 heteroatoms selected from N, O and S as ring members, aryl, 5 to 10 membered heteroaryl having 1-3 heteroatoms selected from N, O and S as ring members, and —C(R^{b1})(R^{b2})C(═O)OR^{b3};
each R^{a1}, R^{a2}, R^{b1}, and R^{b2} is selected from the group consisting of H, D, and C1-C4 alkyl C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocycloalkyl having 1-3 heteroatoms selected from N, O and S as ring members, aryl, and 5 to 10 membered heteroaryl having 1-3 heteroatoms selected from N, O and S as ring members;
each R^{a3} and R^{b3} is independently H, D, C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members; and
R⁵, R⁶ and R⁷ are independently selected from H, —OH, halogen, and R¹²CO₂—, and at least two of R⁵, R⁶ and R⁷ are —OH or R¹²CO₂, wherein R¹² is selected from C1-C8 alkyl, C1-C8 alkoxyl, C1-C8 alkanoyloxyl, C1-C8 alkenyloxyl, C1-C8 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members; wherein any two of the adjacent groups of R⁵, R⁶ and R⁷ can cyclize to form heterocycloalkyl containing 5 to 9 ring members and having 1-3 heteroatoms selected from N, O and S as ring members, each substituted by 0-3 substituents independently selected from D, CN, halogen, —OH, ═O, C1-C4 alkyl and C1-C4 alkoxy.

2. The compound of claim 1, wherein the compound is not (2S,3S,4S,5S,6R)-6-((R)-1,2-dihydroxyethyl)-3,4,5-trihydroxytetrahydro-2H-pyran-2-yl dihydrogen phosphate.

3. The compound of claim 1, wherein the compound of Formula I is represented by Formula Ia

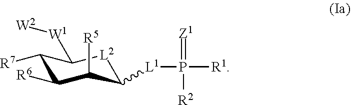

4. The compound of claim 1, wherein the compound of Formula I is represented by Formula Ib

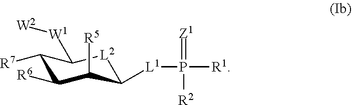

5. The compound of claim 1, wherein the compound of Formula I is represented by Formula Ic

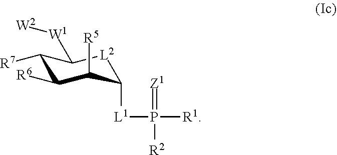

6. The compound according to claim 1, wherein L² is selected from the group consisting of O, S, and CH₂.

7. The compound according to claim 1, wherein L² is O.

8. The compound according to claim 1, wherein L¹ is selected from the group consisting of O, S, CH₂, CHF, and CF₂.

9. The compound according to claim 1, wherein L¹ is O.

10. The compound according to claim 1, wherein Z¹ is O.

11. The compound according to claim 1, wherein R¹ and R² are each —OR^{a} and the R^{a} moieties can combine to form a five or six-membered heterocyclic ring, wherein
the five or six-membered heterocyclic ring is substituted with from 0 to 3 R³ moieties selected from the group consisting of H, D, halogen, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members, wherein the aryl or the 5 or 6 membered heteroaryl are substituted with 0 to 3 R^{3a} substituents selected from the group consisting of halogen and C1-C8 alkyl; or
when two R³ substituents are on adjacent ring vertices of the five or six-membered heterocyclic ring, they can combine to form a fused phenyl ring, which is substituted with from 0 to 3 R⁴ moieties selected from the group consisting of H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members.

12. The compound of claim 11, wherein the combined R^{a} moieties along with the oxygen and phosphorous atoms to which they are attached are represented by Formula iii, 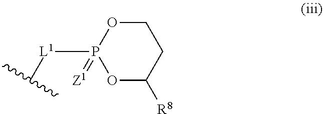
wherein R⁸ is selected from the group consisting of aryl, 3 to 6 membered heterocycloalkyl, and 5 or 6 membered heteroaryl wherein the 3 to 6 membered heterocycloalkyl and the 5 to 10 membered heteroaryl each have 1-3 heteroatoms selected from N, O and S as ring members, and
the wavy line indicates the point of attachment to the rest of the molecule.

13. The compound of claim 11, wherein the combined R^{a} moieties along with the oxygen and phosphorous atoms to which they are attached are represented by Formula ii, 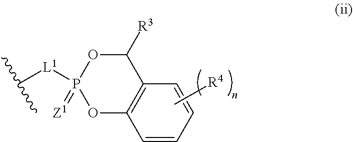
wherein R³ is selected from the group consisting of H, D, C1-C12 alkyl, C1-C12 alkoxyl, C1-C12 haloalkyl, C1-C12 haloalkoxyl, C1-C12 alkenyloxyl, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
each R⁴ is independently selected from H, D, halogen, —OH, C1-C12 alkyl, C1-C12 alkoxyl, C1-C12 haloalkyl, C1-C12 haloalkoxyl, C1-C12 alkenyloxyl, C1-C4 alkylamino, aralkyloxyl, C3-C6 cycloalkyl, 3 to 6 membered heterocyclyoalkyl having 1-3 heteroatoms selected from N, O and S as ring members, aryl, and 5 to 10 membered heteroaryl having 1-3 heteroatoms selected from N, O and S as ring members;
the subscript n is an integer from 1 to 3; and
the wavy line indicates the point of attachment to the rest of the molecule.

14. The compound according to claim 1, wherein R¹ and R² are selected from the group consisting of —OR^{a}, —NR^{b}R^{c}.

15. The compound of claim 14, wherein R¹ and R² combined with the phosphate to which they are attached are represented by Formula i 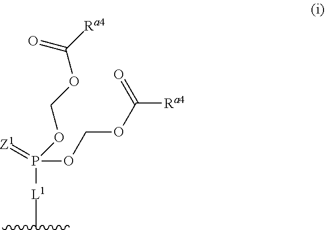
wherein each R^{a4} is each independently selected from C1-C12 alkyl, C1-C12 alkoxyl, C1-C12 alkanoyloxyl, C1-C12 alkenyloxyl, C1-C12 alkylamino, C3-C6 cycloalkyl, 4 to 6 membered heterocycloalkyl having 1-3 heteroatoms selected from N, O and S as ring members, aryl, and 5 to 10 membered heteroaryl having 1-3 heteroatoms selected from N, O and S as ring members, and
the wavy line indicates the point of attachment to the rest of the molecule.

16. The compound of claim 14, wherein R¹ and R² combined with the phosphate to which they are attached are represented by Formula iv 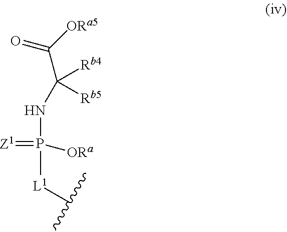
wherein R^{b4} and R^{b5} are optional independently H or D, C1-C4 alkyl, C1-C4 alkoxyl, C1-C4 haloalkyl, C1-C4-haloalkoxyl, C1-C4 alkenyloxyl, aralkyloxyl, C3-C6 cycloalkyl, 3 to 6 membered heterocyclyoalkyll having 1-3 heteroatoms selected from N, O and S as ring members, aryl, and 5 to 10 membered heteroaryl having 1-3 heteroatoms selected from N, O and S as ring members;
R^{a5} is H, D, C1-C12 alkyl, C1-C12 alkoxyl, C1-C12 alkanoyloxyl, C1-C12 alkenyloxyl, C1-C12 alkylamino, aralkyloxyl, C3-C6 cycloalkyl, 4 to 6 membered heterocyclyoalkyll having 1-3 heteroatoms selected from N, O and S as ring members, aryl, and 5 to 10 membered heteroaryl having 1-3 heteroatoms selected from N, O and S as ring members;
R^{a} is H, D, aryl or 3 to 6 ring membered heterocyclyoalkyl having 1-3 heteroatoms selected from N, O and S as ring members, aryl, and heteroaryl containing 5 to 10 ring atoms and having 1-3 heteroatoms selected from N, O and S as ring members, —C1-C4 alkylene-aryl, and —C1-C4 alkylene-5 to 10 membered heteroaryl, wherein the 5 or 10 membered heteroaryl has 1-3 heteroatoms selected from the group consisting of O, N, and S as ring members;
and the wavy line indicates the point of attachment to the rest of the molecule.

17. The compound of claim 14, wherein R¹ and R² combined with the phosphate to which they are attached are represented by Formula v 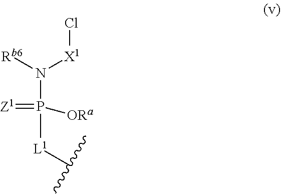
wherein R^{b6} is H, C1-C12 alkyl, C1-C12 alkoxyl, C1-C12 alkanoyloxyl, C1-C12 alkenyloxyl, C3-C6 cycloalkyl, 4 to 6 membered heterocycloalkyl having 1-3 heteroatoms selected from N, O and S as ring members, aryl, 5 to 10 membered heteroaryl having 1-3 heteroatoms selected from N, O and S as ring members;
X¹ is C₃₋₅ alkylene;
and R^{a} is H, D, 3 to 6 membered heterocyclyoalkyl having 1-3 heteroatoms selected from N, O and S as ring members, aryl, and 5 to 10 membered heteroaryl, —C1-C4 alkylene-aryl, and —C1-C4 alkylene-5 to 10 membered heteroaryl, wherein the 5 or 10 membered heteroaryl has 1-3 heteroatoms selected from the group consisting of O, N, and S as ring members;
and the wavy line indicates the point of attachment to the rest of the molecule.

18. The compound of claim 14, wherein the compound is represented by Formula Id 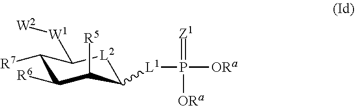
wherein each R^{a} is phenyl.

19. The compound according to claim 1 wherein R⁵, R⁶ and R⁷ are independently selected from —OH, halogen, and R¹²CO₂—, at least two of R⁵, R⁶ and R⁷ are —OH or R¹²CO₂—, and wherein R¹² is selected from C1-C4 alkyl.

20. The compound according to claim 1, wherein W¹ is —C(R¹⁰R¹¹)—,
R¹⁰ and R¹¹ are independently selected from H, D, —OH, halogen, and optionally substituted groups selected from C1-C4 alkyl, C1-C4 alkoxyl, C1-C4 haloalkyl, C1-C4-haloalkoxy, C1-C4 alkenyloxy, aralkyloxy, and R¹²CO₂—,
R¹² is selected from C1-C20 alkyl, C1-C20 alkenyl, C1-C20 alkoxy, C1-C20 alkenyloxy, C1-C20 alkylamino,
wherein the optional substituents for R¹⁰ and R¹¹ are 1-3 substituents independently selected from D, halogen, —OH, ═O, C1-C4 alkyl and C1-C4 alkoxy.

21. The compound according to claim 1, wherein W¹ is —C(R¹⁰R¹¹)—,
R¹⁰ and R¹¹ are independently selected from H, —OH, halogen, and optionally substituted groups selected from C1-C4 alkyl, C1-C4 alkoxyl, C1-C4 haloalkyl, C1-C4-haloalkoxy, C1-C4 alkenyloxy, aralkyloxy, and R¹²CO₂—,
R¹² is selected from C1-C4 alkyl, C1-C4 alkenyl, C1-C4 alkoxy, C1-C4 alkenyloxy, C1-C4 alkylamino,
wherein the optional substituents for R¹⁰ and R¹¹ are 1-3 substituents independently selected from D, halogen, —OH, ═O, C1-C4 alkyl and C1-C4 alkoxy.

22. The compound according to claim 1, wherein W¹ is —C(R¹⁰R¹¹)—,
R¹⁰ and R¹¹ are independently selected from H, —OH, halogen, and R¹²CO₂—,
R¹² is selected from C1-C4 alkyl, C1-C4 alkenyl, C1-C4 alkoxy, C1-C4 alkenyloxy, C1-C4 alkylamino.

23. The compound according to claim 1, wherein W¹ is —C(R¹⁰R¹¹)—, wherein R¹⁰ and R¹¹ are independently selected from H, —OH, halogen, and R¹²CO₂—, and wherein R¹² is selected from C1-C4 alkyl.

24. The compound according to claim 1, wherein W¹ is —C(R¹⁰R¹¹)—, wherein R¹⁰ is H and R¹¹ is selected from —OH and halogen.

25. The compound according to claim 1, wherein W¹ is —C(R¹⁰R¹¹)—, and wherein R¹⁰ is H and R¹¹ is fluoro.

26. The compound according to claim 1, wherein W² is R¹³-Q¹-W³—,
Q¹ is —O—,
W³ is C1 alkylene group,
R¹³ is 1-6 membered oligopeptidyl linked via C-terminal carbonyl group or R¹⁴Q²C(O)—,
Q² is a bond
R¹⁴ is 1-6 membered oligopeptidyl linked via N-terminal N or an optionally substituted group selected from C1-C20 alkyl, C1-C20 alkylenyl, C1-C20 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members, and
R¹⁴ is R¹⁵-Q³-Q⁴-Q⁵-, wherein Q³, Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy,
wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy.

27. The compound according to claim 1, wherein W² is R¹³-Q¹-W³—, wherein Q¹ is —O—; W³ is C1 alkylene group wherein R¹³ is R¹⁴Q²C(O)—, wherein
Q² is a bond,
R¹⁴ is an optionally substituted group selected from C1-C20 alkyl, C1-C20 alkylenyl, C1-C20 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members, and R¹⁴ is R¹⁵-Q³-Q⁴-Q⁵-; wherein Q³, Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy,
wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy.

28. The compound according to claim 1, wherein W² is R¹³-Q¹-W³—, wherein Q¹ is —O—; W³ is C1 alkylene group wherein R¹³ is R¹⁴Q²C(O)—; wherein Q² is a bond; R¹⁴ is R¹⁵-Q³-Q⁴-Q⁵-; wherein Q³, Q⁴ and Q⁵ are independently selected from a bond, aryl, heteroaryl containing 5 to 6 ring atoms, C3-C6 cycloalkyl and heterocyclyl containing 4 to 6 ring members and having 1-3 heteroatoms selected from N, O and S as ring members, and at least one of Q³, Q⁴ and Q⁵ is not a bond; R¹⁵ is an optionally substituted group selected from C1-C18 alky and C1-C18 alkoxy, wherein the optional substituents for R¹⁴ and R¹⁵ are 1-3 substituents independently selected from halogen, —OH, —CO₂H, C1-C4 alkyloxycarbony, C1-C4 alkyl, C1-C4 haloalkyl, C1-C4 alkoxy, C1-C4 haloalkoxy C3-C6 cycloalkyl and C3-C6 cycloalkyloxy.

29. The compound according to claim 1, wherein W² is R¹³-Q¹-W³—, wherein Q¹ is —O—; W³ is C1 alkylene group wherein R¹³ is R¹⁴Q²C(O)—; wherein Q² is a bond; R¹⁴ is an optionally substituted group selected from C1-C20 alkyl, C1-C20 alkylenyl, C1-C20 alkylamino, C3-C6 cycloalkyl, heterocycloalkyl containing 3 to 6 ring members and having 1-3 heteroatoms selected from N, O and S as ring members, aryl, and heteroaryl containing 5 to 10 ring atoms and having 1-3 heteroatoms selected from N, O and S as ring members.

30. The compound according to claim 1, wherein W² is R¹³-Q¹-W³—, wherein Q¹ is —O—; W³ is C1 alkylene group wherein R¹³ is R¹⁴Q²C(O)—; wherein Q² is a bond; R¹⁴ is an optionally substituted group selected from C1-C20 alkyl, C1-C20 alkylenyl, C1-C20 alkylamino.

31. The compound according to claim 1, wherein W² is R¹³-Q¹-W³—, wherein Q¹ is —O—; W³ is C1 alkylene group wherein R¹³ is R¹⁴Q²C(O)—; wherein Q² is a bond; R¹⁴ is an optionally substituted group selected from C1-C4 alkyl.

32. The compound according to claim 1, and/or a stereoisomer, a stable isotope, prodrug or a pharmaceutically acceptable salt thereof selected from Table 1.

33. A pharmaceutical composition comprising a compound of claim 1 and a pharmaceutically acceptable carrier.

34. A method for activating ALPK1, the method comprising administering an effective amount of a compound or a pharmaceutically acceptable salt of claim 1.

35. A method for modulating an immune response in a subject in need of such treatment, the method comprising administering to the subject an effective amount of a compound or a pharmaceutically acceptable salt of claim 1.

36. A method for treating cancer in a subject in need of such treatment, the method comprising administering to the subject an effective amount of a compound or a pharmaceutically acceptable salt of claim 1.

37. A method for potentiating an immune response to a target antigen in a subject, the comprising administering to the subject an effective amount of a compound or a pharmaceutically acceptable salt of claim 1.

38. A method for treating a disease or disorder amendable to treatment by activation of NFkB, p38, and JNK cell signaling pathways in cells of a subject, the method comprising administering to the subject an effective amount of a compound or a pharmaceutically acceptable salt of claim 1.

39. A method for treating or preventing a disease or disorder caused by an infectious agent selected from a bacteria, virus, or parasite in a subject in need thereof, the comprising administering to the subject an effective amount of a compound or a pharmaceutically acceptable salt of claim 1.

40. The method of claim 35, wherein modulating an immune response is selected from activation of innate immunity and activation of adaptive immunity.

41. The method of claim 36, wherein the cancer is selected from soft tissue sarcoma, breast cancer, head and neck cancer, melanoma, cervical cancer, bladder cancer, hematologic malignancy, glioblastoma, pancreatic cancer, prostate cancer, colon cancer, breast cancer, renal cancer, lung cancer, merkel cell carcinoma, small intestine cancer, thyroid cancer, acute myelogenous leukemia (AML), acute lymphocytic leukemia (ALL), chronic lymphocytic leukemia (CLL), chronic myelogenous leukemia (CML), gastric cancer, gastrointestinal stromal tumors, non-Hodgkins lymphoma, Hodgkins lymphoma, liver cancer, leukemia, lymphoma, T-cell lymphoma, brain cancer, and multiple myeloma.

42. The method of claim 37, wherein the target antigen is an antigen of an infectious agent selected from the group consisting of adenovirus, Coxsackie B virus, cytomegalovirus, eastern equine encephalitis virus, ebola virus, enterovirus 71, Epstein-Barr virus, Haemophilus influenzae type b (Hib), hepatitis C virus (HCV), herpes virus, human immunodeficiency virus (HIV), human papillomavirus (HPV), hookworm, Marburg virus, norovirus, respiratory syncytial virus (RSV), rotavirus, Salmonella typhi, Staphylococcus aureus, Streptococcus pyogenes, varicella, West Nile virus, Yersinia pestis, and Zika virus.

43. The method of claim 37, wherein the compound, acts as a vaccine adjuvant for a vaccine in the treatment or prevention of anthrax, caries, Chagas disease, dengue, diphtheria, ehrlichiosis, hepatits A or B, herpes, seasonal influenza, Japanese encephalitis, leprosy, lyme disease, malaria, measles, mumps, meningococcal disease, including meningitis and septicemia, Onchocerciasis river blindness, pertussis (whooping cough), pneumococcal disease, polio, rabies, rubella, schistosomiasis, severe acute respiratory syndrome (SARS), shingles, smallpox, syphilis, tetanus, tuberculosis, tularemia, tick-borne encephalitis virus, typhoid fever, trypanosomiasis, yellow fever, or visceral leishmaniasis.

44. The method of claim 38, wherein the disease or disorder is selected from tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, ankylosing spondylitis bullous diseases, actinic keratoses, ulcerative colitis, Crohn's disease, alopecia areata, and diseases and disorders caused by the hepatitis C virus (HCV), the hepatitis B virus (HBV), or the human immunodeficiency virus (HIV).

45. The method of claim 39, wherein the infectious agent is a bacteria.

46. The method of claim 39, wherein the infectious agent is a virus.

47. The method of claim 39, wherein the infectious agent is a parasite.

48. The method of claim 45, wherein the bacteria is a Gram-negative or a Gram-positive bacteria.

49. The method of claim 48, wherein the Gram-negative bacteria is selected from the group consisting of Acinetobacter baumanii, Aggregatobacter actinomycetemcomitans, Bartonella bacilliformis, Bartonella henselae, Bartonella quintana, Bifidobacterium Borrelia, Bortadella pertussis, Brucella sp, Burkholderia cepacis, Burkholderia pseudomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup I, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B. typhus,
Salmonella dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus and Yersinia pestitis.

50. The method of claim 48, wherein the Gram-positive bacteria selected from the group consisting of Actinomycetes, Bacillus anthracis, Bacillus subtilis, Clostridium tetani, Clostridium perfingens, Clostridium botulinum, Clostridium tetani, Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacterium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, and Streptococcus mutants.

51. The method of claim 46, wherein the virus is selected from the group consisting of ebolavirus, hepatitis B virus, hepatitis C virus, herpes simplex virus, human immunodeficiency virus (HIV), human papillomavirus (HPV-6, HPV-11), human SARS coronavirus, influenza A virus, influenza B virus, influenza C virus, measles virus, rabies virus, poliovirus, SARS corona virus, and yellow fever virus.

52. The method of claim 47, wherein the parasite is selected from the group consisting of Acanthamoeba spp, American tryppanosomiasis, Balamuthia mandrillaris, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleriafowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

53. The method of claim 35, further comprising administering to the subject one or more additional therapeutic agents or immune modulators, and combinations thereof.

54. The method of claim 53, wherein the one or more additional therapeutic agents is selected from an anti-microbial agent, such as an anti-bacterial agent, an anti-viral agent, or an anti-parasitic agent, an anti-cancer agent, or a therapeutic agent for the treatment of tuberculosis, meningitis, pneumonia, ulcer, sepsis, rhinitis, asthma, allergy, COPD, inflammatory bowel disease, arthritis, obesity, radiation-induced inflammation, psoriasis, atopic dermatitis, non-alcoholic steatohepatitis (NASH), Alzheimer's disease, systemic lupus, erythematosus (SLE), autoimmune thyroiditis (Grave's disease), multiple sclerosis, and ankylosing spondylitis bullous diseases.

55. The method of claim 53, wherein the one or more additional immune modulators is selected from the group consisting of an inhibitor or antagonist of an immune checkpoint regulator, a vaccine, preferably a vaccine against an immune checkpoint regulator, an immune stimulatory molecule, an agonist of an immune co-stimulatory molecule, a recombinant protein, and a T cell, preferably a chimeric antigen receptor T (CAR-T) cell.

56. The method of claim 55, wherein the immune checkpoint regulator is selected from the programed cell death 1 (PD-1) receptor (CD279), a ligand of PD-1 (e.g., PD-L1), cytotoxic T-lymphocyte associated protein 4 (CTLA4), tumor necrosis factor receptor superfamily member 9 (alternatively TNFRSF9, 4-1BB) and 4-1BB ligands, tumor necrosis factor receptor superfamily member 4 (alternatively TNFRSF4, OX40) and OX40 ligands, glucocorticoid-induced TNFR-related protein (GITR), Tumor Necrosis Factor Receptor Superfamily Member 7 (alternatively TNFRSF7, cluster of differentiation 27, CD27), TNFRSF25 and TNF-like ligand 1A (TL1A), TNF Receptor Superfamily Member 5 (alternatively TNFRSF5, CD40) and CD40 ligand, Herpesvirus entry mediator (HVEM)-tumor necrosis factor ligand superfamily member 14 (alternatively TNFSF14, LIGHT)-lymphotoxin alpha (LTA), herpesvirus entry mediator-(HVEM)-B- and T-lymphocyte attenuator (BTLA)-CD160 (alternatively TNFSF14), lymphocyte activating gene 3 (LAG3), T-cell immunoglobulin and mucin-domain containing-3 (TIM3), sialic-acid-binding immunoglobulin-like lectins (SIGLECs), inducible T-cell costimulator (ICOS) and ICOS ligand, B7-113 (B7 family, alternatively CD276), V-set domain-containing T-cell activation inhibitor 1 (VTCN1, alternatively B7-114), V-Type immunoglobulin domain-containing suppressor of T-cell activation (VISTA), human endogenous retrovirus-H long terminal repeat-associating protein 2 (HHLA2)-transmembrane and Immunoglobulin domain containing 2 (TMIGD2), butyrophilins, natural killer cell receptor 2B4 (alternatively NKR2B4, CD244) and B-Cell Membrane Protein (CD48), T-Cell Immunoreceptor with Immunoglobulin (Ig) and immunoreceptor tyrosine-based inhibition motif domains (TIGIT) and Poliovirus receptor (PVR) family members, killer-cell immunoglobulin-like receptors (KIRs), Immunoglobulin-like transcripts (ILTs) and leukocyte immunoglobulin-like receptor (LIRs), natural killer group protein 2 member D (NKG2D) and natural killer group protein 2 member A (NKG2A), major histocompatibility complex (MHC) class I polypeptide-related sequence A (MICA) and MHC class I polypeptide-related sequence B (MICB), natural killer cell receptor 2B4 (CD244), colony stimulating factor 1 receptor (CSF1R), indoleamine 2,3-dioxygenase (IDO), transforming growth factor beta (TGFβ), Adenosine-ecto-nucleotidase triphosphate diphosphohydrolase 1 (CD39)-5′-nucleotidase (CD73), C—X—C motif chemokine receptor 4 (CXCR4) and C—X—C motif chemokine ligand 12 (CXCL12), phosphatidylserine, signal regulatory protein alpha (SIRPA) and integrin associated protein (CD47), vascular endothelial growth factor (VEGF), and neuropilin.

57. The method of claim 53, wherein the one or more additional immune modulators is a vaccine.

58. The method of claim 57, in a method for treating cancer, wherein the vaccine is a vaccine against a tumor antigen.

59. The method of claim 58, wherein the tumor antigen is selected from glycoprotein 100 (gp100), mucin 1 (MUC1), and melanoma-associated antigen 3 (MAGEA3).

60. The method of claim 53, wherein the one or more additional immune modulators is a T cell, preferably a chimeric antigen receptor T cell.

61. The method of claim 53, wherein the one or more additional immune modulators is a recombinant protein, preferably selected from granulocyte-macrophage colony-stimulating factor (GM-CSF), interleukin 7 (IL-7), IL-12, IL-15, IL-18, and IL-21.

62. The method of claim 35, wherein the composition comprises a compound selected from the group consisting of

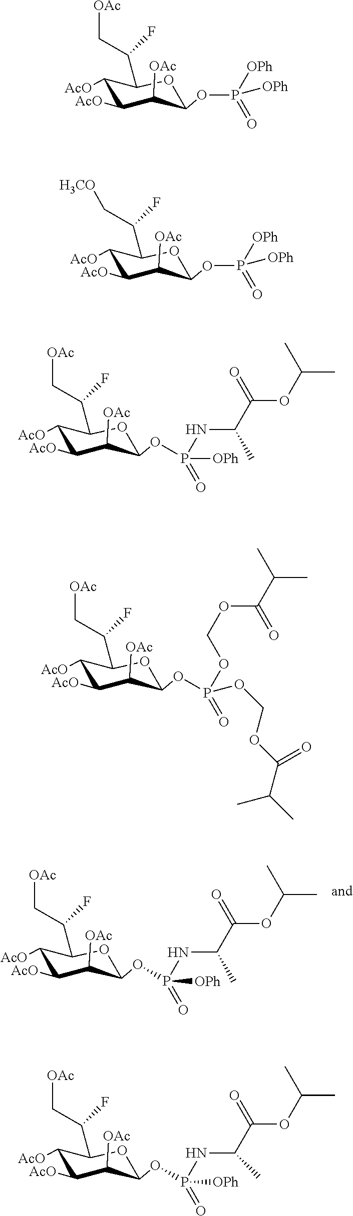

63. The method of claim 53, wherein the one or more additional therapeutic agents or immune modulators is a PD-1/PD-L1 inhibitor.

64. The method of claim 63, wherein the PD-1/PD-L1 inhibitor is selected from the group consisting of nivolumab, pembrolizumab, pidilizumab, BMS-936559, atezolizumab, durvalumab, and avelumab.

65. The method of claim 36, wherein the cancer is selected from advanced melanoma, non-small cell lung cancer, renal cell carcinoma, bladder cancer, Hodgkin's lymphoma, liver cancer, gastric cancer, colon cancer, breast cancer, non-Hodgkin's lymphoma, prostate cancer, head and neck cancer, thyroid cancer, brain cancer, acute myeloid leukemia (AML), merkel cell carcinoma, multiple myeloma, cervical cancer, and sarcoma.

66. A method for treating cancer in a subject in need of such treatment, the method comprising administering to the subject a composition comprising a compound of claim 1, and an immune modulator selected from one or more of an inhibitor or antagonist of an immune checkpoint regulator, an immune stimulatory molecule, and an agonist of an immune co-stimulatory molecule.

67. The method of claim 66, wherein the inhibitor or antagonist of an immune checkpoint regulator is a PD-1/PD-L1 inhibitor.

68. The method of claim 67, wherein the PD-1/PD-L1 inhibitor is selected from the group consisting of nivolumab, pembrolizumab, pidilizumab, BMS-936559, atezolizumab, durvalumab, and avelumab.

69. The method of claim 66, wherein the immune modulator is selected from interferon alpha (INFα), a stimulator of interferon genes (“STING”) agonist, a TLR agonist (e.g., resquimod), and an anti-OX40 (CD134) agonist antibody.

70. The method of claim 66, wherein immune modulator is an agonist of an immune co-stimulatory molecule.

71. The method of claim 70, wherein the agonist of an immune co-stimulatory molecule is an anti-OX40 (CD134) agonist antibody.

72. A method for treating a liver disease or disorder in a subject in need of such treatment, the method comprising administering a compound or a pharmaceutically acceptable salt of claim 1 to the subject.

73. The method of claim 72, wherein the liver disease or disorder is selected from liver cancer, non-alcoholic steatohepatitis (NASH), and a disease or disorder caused by infection with the hepatitis C virus (HCV) or the hepatitis B virus (HBV).

74. The method of claim 35, wherein the subject is a vertebrate.

75. The method of claim 35, wherein the subject is a human.

76. A vaccine composition or vaccine adjuvant composition comprising a compound or a pharmaceutically acceptable salt of claim 1.

77. A pharmaceutical composition, a vaccine composition, or a vaccine adjuvant composition comprising a compound or a pharmaceutically acceptable salt of claim 1.
